(19) United States
(12) Patent Application Publication
(10) Pub. No.: US 2003/0082595 A1
(43) Pub. Date: May 1, 2003

(54) Nucleic acids of aspergillus fumigatus encoding industrial enzymes and methods of use

(76) Inventors: Bo Jiang, Montreal (CA); Reginald Storms, Beaconsfield (CA); Terry Roemer, Montreal (CA); Howard Bussey, Westmount (CA)

Correspondence Address: PENNIE AND EDMONDS, 1155 AVENUE OF THE AMERICAS, NEW YORK, NY, 100362711

(21) Appl. No.: 10/213,990

(22) Filed: Aug. 5, 2002

Related U.S. Application Data
(60) Provisional application No. 60/309,870, filed on Aug. 3, 2001.

(51) Int. Cl. C12Q 1/68; C07H 21/04; C12P 19/04; C08B 37/00; C12N 9/02; C12N 9/24; C12P 21/02; C12N 5/06

(52) U.S. Cl. 435/6; 435/69.1; 435/189; 435/200; 435/101; 435/320.1; 435/325; 536/23.2; 536/123

(57) ABSTRACT

The present invention provides nucleotide sequences of Aspegillus fumigatus that encode proteins which exhibit enzyme activities. Vectors, expression constructs, and host cells comprising the nucleotide sequences of the enzyme genes are also provided. The invention further provides methods for producing the enzymes, and methods for modifying the enzymes in order to improve their desirable characteristics. The activities displayed by the enzymes of the invention include those of a tannase, cellulase, glucose oxidase, glucoamylase, phytase, β-galactosidases, invertase, lipase, α-amylase, laccase, polygalacturonase or xylanase. The enzymes of the invention can be used in a variety of industrial processes. Enzymatically active compositions in various forms as well as antibodies to the enzymes and fragments thereof, are also provided.

### 1. INTRODUCTION

The present invention is directed toward isolated nucleic acids of Aspergillus fumigatus that encode enzymes with industrial applications, and methods of uses.

### 2. BACKGROUND OF THE INVENTION

Enzymatic processes enable natural raw materials to be refined and/or converted into useful intermediates or finished products. Historically, enzymatic processes had been used for the production of foodstuffs and flavorings. During traditional koji fermentation in China and Japan, various filamentous fungi such as Aspergillus oryzae and Aspergillus sojae have been used to make soy sauce, miso (soyabean paste) and sake wine. Jokichi Takamine was awarded U.S. Pat. No. 525,823 in 1894 for the first microbial enzyme, an α-amylase from A. oryzae, to be manufactured for commerce.

In 1960's and 1970's, several different enzymes, such as amyloglucosidases (AMG by Novo Nordisk, DIAZYME by Solvay) and glucose isomerases (SPEZYME by Genencor, SWEETZYME by Novo Nordisk) became widely used for converting starch from various natural sources into a range of syrups. Many other types of enzymes are now being used in the wine and juice industries, bakeries, and in the cheese industry. See Bigelis, R. “Food enzymes” in “Biotechnology of filamentous fungi”, ed. by Finkelstein and Ball, 1992, Butterworth-Heinemann.

Enzymes are also extensively used in the textile and leather industries which uses various enzymes to desize textile fibers and to make soft and supple leather from rawhides. In the detergent industry, several generations of proteases with desirable properties such as a high pH optimum and stability have been developed in the last 30 years, e.g., ESPERASE in 1974 by Novo Nordisk, and OPTICLEAN in 1982 by Solvay. Lipase and cellulase type detergent enzymes have also been developed, e.g., CELLUZYME and LIPOLASE both by Novo Nordisk. In recent years, enzymes for use in detergents based on genetic engineering techniques were introduced, e.g., SUBTILISIN NOVO (Genencor), and bleach-stable high pH proteases MAXAPEM by IBIS.

Worldwide consumption of industrial enzymes amounted to approximately $720 million in 1990. The growth in volume of the enzyme business from 1980 to 1990 was estimated to be 5-10% per year. Overall, the starch conversion and detergent industries are by far the most important consumers. The other major uses are in the dairy, textile, and alcohol-making industries.

The synthesis of polymers, pharmaceuticals, natural products and agrochemicals is often hampered by expensive processes which produce harmful byproducts and which suffer from low selectivity with respect to optical isomers. Enzymes have a number of remarkable advantages which can overcome many of the current problems in catalysis: they act on single functional groups, they distinguish between similar functional groups on a single molecule, they distinguish between enantiomers, and they function at very low mole fractions in reaction mixtures. Because of the specificity of their actions, enzymes present a unique solution to achieve selective transformations which are often extremely difficult to duplicate chemically. The elimination of the need for protection groups, selectivity, the ability to carry out multi-step transformations in a single reaction vessel, has led to an increased demand for enzymes in chemical and pharmaceutical industries. A current limitation to more widespread industrial use is primarily due to the relatively small number of commercially available enzymes.

The industrial use of enzymes is also an important contribution to the development of environment-friendly technology. They replace conventional chemical-based technologies and energy-intensive manufacturing processes. They originate from natural biological systems and are therefore totally biodegradable. Generally, enzymatic processes require less energy, less equipment or fewer chemicals.

As the need for better catalysts and the interest in using environment-friendly processes grow, there is an emerging need for more effective and robust enzymes for a variety of industries. So far, enzymes of commercial interest have been obtained from several Aspergillus species, such as, A. niger, A. oryzae, A. awamori, A. alliaceus, A. usamii, A. ficum, A. saitoi, and A. melleus.

Aspergillus fumigatus is a saprophytic fungus that plays an essential role in recycling environmental carbon and nitrogen. Its natural ecological niche is the soil, wherein it survives and grows on organic debris. Although this species is not the most prevalent fungus in the world, it is one of the most ubiquitous of those with airborne conidia. Inhalation of the conidia by an immunosuppressed individual often leads to an opportunistic infection with A. fumigatus which is severe and can be fatal. It is the most common etiological agent of Aspergillus infections in humans. However, unlike the other Aspergillus species, very little is known about the enzymes of A. fumigatus. U.S. Pat. No. 4,593,005 discloses amylolytic enzymes from an Aspergillus strain that share some morphological characteristics with A. fumigatus.

The present invention takes a genomics approach to identify enzymes in Aspergillus fumigatus that can be used in industrial processes. Citation or discussion of a reference herein shall not be construed as an admission that such is prior art to the present invention.

### 3. SUMMARY OF THE INVENTION

The present invention provides the nucleotide sequences of twenty four enzyme genes of Aspergillus fumigatus. The enzyme genes encode a protein with an enzyme activity that is either in use in an industry or of interest to an industry. The genomic sequences of the invention that encode the enzymes are identified primarily by comparison of nucleotide sequences of A. fumigatus genomic DNA and the nucleotide sequences of known enzyme genes of other microorganisms. Prior to this invention, the nucleotide sequences of these A. fumigatus genes, the reading frames, the positions of exons and introns, the structure of the enzymes, and their potential usefulness in various industries, such as those involved in the making of food and feed, beverages, textiles and detergents, were not known. Without limitation, the polynucleotides of the enzyme genes can be used to express recombinant enzymes for characterization, modifications or industrial uses; to compare with the nucleic acid sequence of Aspergillus fumigatus to identify duplicated genes or paralogs having the same or similar biochemical activity and/or function; to compare with nucleic acid sequences of other related or distant fungal organisms to identify potential orthologous enzyme genes; for selecting and making oligomers for attachment to a nucleic acid array for examination of expression patterns; and to raise anti-protein antibodies using nucleic acid immunization techniques. The sequence information provided herein can also form a basis for the design and testing of genetically modified enzymes which possess desirable chemical and physical characteristics.

In one embodiment, the invention provides isolated nucleic acids that encode tannases (SEQ ID NO: 1, 2, 4, and 5), a cellulase (SEQ ID NO: 7 and 8), glucose oxidases (SEQ ID NO: 10, 11, 13, 14, 16, and 17), glucoamylases (SEQ ID NO: 19, 20, 31, 32, 52 and 53), a phytase (SEQ ID NO: 22 and 23), β-galactosidases (SEQ ID NO: 25, 26, 28, and 29), a sucrase or invertase (SEQ ID NO: 34 and 35), a lipase (SEQ ID NO: 37 and 38), α-amylases (SEQ ID NO: 40, 41, 43, 44, 46, and 47), a laccase (SEQ ID NO: 49, and 50), polygalacturonases (SEQ ID NO: 55, 56, 58, 59, 61 and 62), and xylanases (SEQ ID NO: 64, 65, 67, 68, 70 and 71). For each gene of the invention, an open reading frame (ORF) sequence was derived manually from the respective genomic sequence by deleting predicted intron sequences and splicing together exon sequences. Vectors, expression vectors, and host cells comprising the enzyme genes are also encompassed.

In another embodiment, the invention provides deduced amino acid sequences of enzymes that are predicted from the ORF sequences of the enzyme genes. Based on the sequence conservation displayed between the Aspergillus fumigatus genes of the invention and their homologs in other fungi, it is predicted that the polypeptides encoded by these A. fumigatus genes exhibit enzymatic activities similar to their homologs. The amino acid sequences of the invention correspond to those of tannases (SEQ ID NO: 3 and 6), cellulase (SEQ ID NO: 9), glucose oxidases (SEQ ID NO: 12, 15, and 18), glucoamylases (SEQ ID NO: 21, 33 and 57), phytase (SEQ ID NO: 24), β-galactosidases (SEQ ID NO: 27 and 30), sucrase or invertase (SEQ ID NO: 36), lipase (SEQ ID NO: 39), α-amylases (SEQ ID NO: 42, 45, and 48), laccase (SEQ ID NO: 51), polygalacturonases (SEQ ID NO: 57, 60 and 63), and xylanases (SEQ ID NO: 66, 69, and 72). The biological activities of the gene products encoded by the Aspergillus fumigatus enzyme genes of the invention can be predicted and confirmed by the outcome of their enzymatic actions on substrates commonly encountered by the fungus in its natural habitats or synthetic substrates. Generally, the enzymes of the invention can be used in various methods for modulating the amounts of enzyme substrates and products in a composition. Enzymatically active compositions in various forms as well as antibodies to the enzymes and fragments thereof, are also provided.

Any or all of these utilities are capable of being developed into a kit for commercialization either as research products or as supplies for industrial uses. The kits may comprise polynucleotides and/or polypeptides corresponding to one or more A. fumigatus enzyme genes of the invention, antibodies, and/or other reagents.

Various publications and patents are cited hereinbelow, the disclosures of which are incorporated by reference in their entireties.

### 4. BRIEF DESCRIPTION OF THE DRAWINGS

FIG. 1: Table 1 lists the sequence identifiers of the genomic and coding sequences of the enzyme genes of the invention, and the amino acid sequences of the encoded polypeptides.

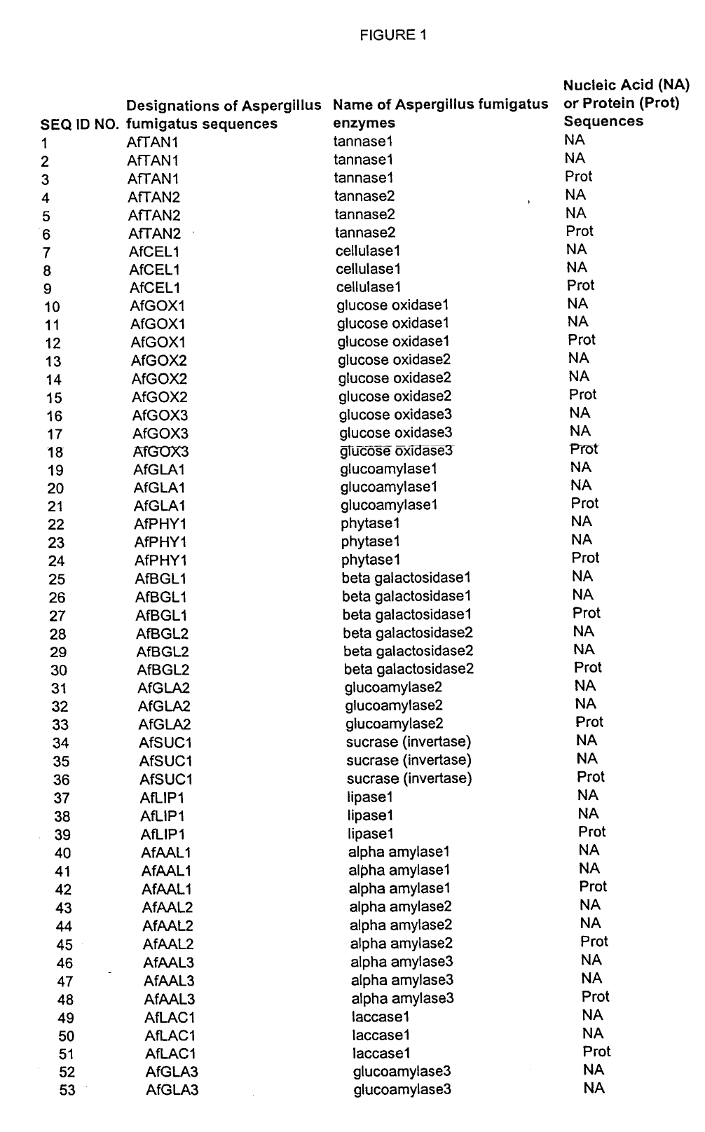

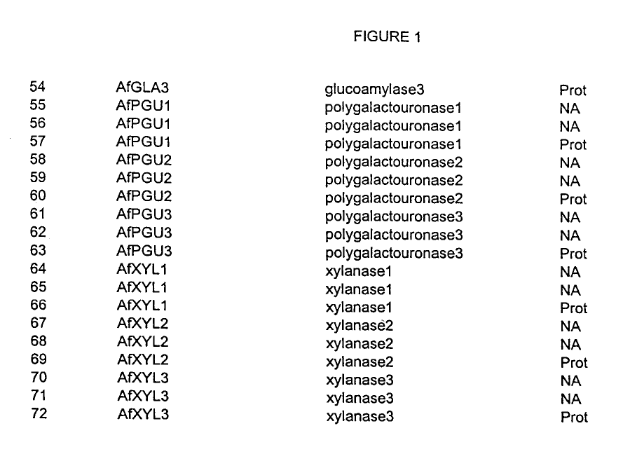

### 5. DETAILED DESCRIPTION OF THE INVENTION

Described hereinbelow are the enzyme genes of the invention, their identification, characterization, modification, and methods of use in various industrial processes. All of the publications and patents cited in this section are incorporated by reference in their entireties.

5.1. Identification of Aspergillus fumigatus Enzyme Genes

The nucleotide sequences of Aspergillus fumigatus genomic DNA was obtained by a whole-genome random shotgun DNA sequencing effort. The genomic DNA was prepared from an isolate of A. fumigatus CEA 10 which was isolated from the infected lung tissue of a human aspergillosis patient. The genomic DNA was sheared mechanically into fragments, enzymatically treated to generate blunt ends, and cloned into E. coli pUC 19- and pBR322-based plasmids to form genomic DNA libraries. Average insert sizes of the pUC19-based genomic DNA library clones were about 2 kb and the plasmids were present in high copy numbers in E. coli cells. The other two genomic DNA libraries of pBR322-based clones contain inserts of about 10 kb and about 50 kb respectively. The colonies of genomic clones were transferred robotically to 384-well titre plates; and plasmid DNA templates for dideoxy DNA sequencing reactions were prepared by standard method based on alkaline lysis of cells and isopropanol precipitation of DNA. DNA sequencing reactions were carried out using standard M13 forward and reverse primers and ABI-Prism BigDye terminator chemistry (Applied Biosystems), and analyzed using the capillary array sequencer ABI PRISM 3700 DNA Analyzer (Applied Biosystems). The nucleotide sequences generated were trimmed to discard errors, and assembled to form contigs and scaffolds by the software algorithms developed for sequencing the human genome. For a detailed description of the methodologies of the sequencing reactions and sequence analysis, see Venter et al., 2001, Science 291:1304 and; Myers et al., 2000, Science 287:2196. The set of nucleotide sequence data used in the present invention has an estimated 10×coverage of the A. fumigatus genome.

The nucleotide sequences were initially annotated by software programs, such as Genescan and Glimmer M (The Institute of Genome Research, Rockville, Md.), which can identify putative coding regions, introns, and splice junctions. Further automated and manual curation of the nucleotide sequences were performed to refine and establish precise characterization of the coding regions and other gene features.

5.2. Enzyme Genes

5.2.1. Nucleic Acid Molecules of Aspergillus fumigatus

Described herein are the nucleic acid molecules of the invention that encode enzymes of industrial interest.

As used herein, the terms “gene” and “recombinant gene” refer to nucleic acid molecules or polynucleotides comprising a nucleotide sequence encoding a polypeptide or a biologically active ribonucleic acid (RNA). The term can further include nucleic acid molecules comprising upstream, downstream, and/or intron nucleotide sequences. The term “open reading frame (ORF),” means a series of nucleotide triplets coding for amino acids without any termination codons and the triplet sequence is translatable into protein using the codon usage information appropriate for a particular organism.

The term “nucleotide sequence” refers to a heteropolymer of nucleotides, including but not limited to ribonucleotides and deoxyribonucleotides, or the sequence of these nucleotides. The terms “nucleic acid” and “polynucleotide” are also used interchangeably herein to refer to a heteropolymer of nucleotides, which may be unmodified or modified DNA or RNA. For example, polynucleotides can be single-stranded or double-stranded DNA, DNA that is a mixture of single-stranded and double-stranded regions, hybrid molecules comprising DNA and RNA with a mixture of single-stranded and double-stranded regions. In addition, the polynucleotide can be composed of triple-stranded regions comprising DNA, RNA, or both. A polynucleotide can also contain one or modified bases, or DNA or RNA backbones modified for nuclease resistance or other reasons. Generally, nucleic acid segments provided by this invention can be assembled from fragments of the genome and short oligonucleotides, or from a series of oligonucleotides, or from individual nucleotides, to provide a synthetic nucleic acid.

The term “recombinant,” when used herein to refer to a polypeptide or protein, means that a polypeptide or protein is derived from recombinant (e.g., microbial or mammalian) expression systems. “Microbial” refers to recombinant polypeptides or proteins made in bacterial or fungal expression systems. Polypeptides or proteins expressed in most bacterial systems, e.g., E. coli, will be free of glycosylation modifications; polypeptides or proteins expressed in fungi will be glycosylated.

The term “expression vehicle or vector” refers to a plasmid, a phage, a virus, an artificial replicating sequence (ARS) or an artificial chromosome for expressing a polypeptide from a nucleotide sequence. An expression vehicle can comprise a transcriptional unit, also referred to as an expression construct, comprising an assembly of (1) a genetic element or elements having a regulatory role in gene expression, for example, promoters or enhancers, (2) a structural or coding sequence which is transcribed into RNA, mRNA and translated into protein, and which is operably linked to the elements of (1); and (3) appropriate transcription initiation and termination sequences. “Operably linked” refers to a link in which the regulatory regions and the DNA sequence to be expressed are joined and positioned in such a way as to permit transcription, as well as translation of the transcripts. Structural units intended for use in fungal or eukaryotic expression systems preferably include a leader or transport sequence enabling extracellular secretion of translated protein by a host cell or targeting of the protein to specific organelle(s). Alternatively, where a recombinant protein is expressed without a leader or transport sequence, it may include an N-terminal methionine residue. This residue may or may not be subsequently cleaved from the expressed recombinant protein to provide a final product.

The term “recombinant host cells” means cultured cells which comprises a recombinant transcriptional unit, and will express heterologous polypeptides or proteins, and RNA encoded by the DNA segment or synthetic gene in the recombinant transcriptional unit. Such recombinant host cells either have stably integrated a recombinant transcriptional unit into chromosomal DNA or carry stably the recombinant transcriptional unit extrachromosomally. This term also means host cells which have stably integrated a recombinant genetic element or elements having a regulatory role in gene expression, for example, promoters or enhancers. This term include host cells which maintains the recombinant transcriptional unit and/or express the heterologus proteins or RNA transiently. Recombinant expression systems as defined herein will express RNA, polypeptides or proteins endogenous to the cell upon induction of the regulatory elements linked to the endogenous DNA segment or gene to be expressed. The cells can be prokaryotic or eukaryotic.

The term “polypeptide” refers to the molecule formed by joining amino acids to each other by peptide bonds, and may contain amino acids other than the twenty commonly used gene-encoded amino acids. The term “active polypeptide” refers to those forms of the polypeptide which retain the enzymatic, biologic and/or immunologic activities of any naturally occurring polypeptide. The term “naturally occurring polypeptide” refers to polypeptides produced by cells that have not been genetically engineered and specifically contemplates various polypeptides arising from post-translational modifications of the polypeptide including, but not limited to, proteolytic processing, acetylation, carboxylation, glycosylation, phosphorylation, lipidation and acylation.

The term “isolated” as used herein refers to a nucleic acid or polypeptide separated from at least one macromolecular component (e.g., nucleic acid or polypeptide) present with the nucleic acid or polypeptide in its natural source. In one embodiment, the polynucleotide or polypeptide constitutes at least 95% by weight, more preferably at least 99.8% by weight, of the indicated biological macromolecules present (but water, buffers, and other small molecules, especially molecules having a molecular weight of less than 1000 daltons, can be present).

Encompassed by the present invention are genomic nucleotide sequences and coding sequences of genes that encode enzymes of Aspergillus fumigatus of industrial interest. Accordingly, in one embodiment, SEQ ID NO: 2, 5, 8, 11, 14, 17, 20, 23, 26, 29, 32, 35, 38, 41, 44, 47, 50, 53, 56, 59, 62, 65, 68, and 71 are provided each of which identifies a nucleotide sequence of the opening reading frame (ORF) of an identified enzyme gene. In another embodiment, the genomic sequences of the enzyme genes identified by SEQ ID NO: 1, 4, 7, 10, 13, 16, 19, 22, 25, 28, 31, 34, 37, 40, 43, 46, 49, 52, 55, 58, 61, 64, 67, and 70 are provided.

The DNA sequences of the invention were generated by sequencing reactions and may contain minor errors which may exist as misidentified nucleotides, insertions, and/or deletions. However, such minor errors, if present, should not disturb the identification of the sequences as a gene of A. fumigatus that encodes an enzyme of industrial interest, and are specifically encompassed within the scope of the invention.

The enzyme genes listed in Table 1 can be obtained using cloning methods well known to those of skill in the art, and include but are not limited to the use of appropriate probes to detect the genes within an appropriate cDNA or gDNA (genomic DNA) library (See, for example, Sambrook et al., 1989, Molecular Cloning: A Laboratory Manual, Cold Spring Harbor Laboratories,). Probes for the sequences identified herein can be synthesized based on the DNA sequences disclosed herein in SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70.

As used herein, “enzyme gene” refers to (a) a gene comprising at least one of the nucleotide sequences and/or fragments thereof that are set forth in SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70; (b) any nucleotide sequence or fragment thereof that encodes the amino acid sequence that are set forth in SEQ ID NO: 3, 6, 9, 12, 15, 18, 21, 24, 27, 30, 33, 36, 39, 42, 45, 48, 51, 54, 57, 60, 63, 66, 69, and 72; (c) any nucleotide sequence that hybridizes to the complement of the nucleotide sequences set forth in SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70 under medium stringency conditions, e.g., hybridization to filter-bound DNA in 6×sodium chloride/sodium citrate (SSC) at about 45° C. followed by one or more washes in 0.2×SSC/0.1% SDS at about 50 to 65° C., or under highly stringent conditions, e.g., hybridization to filter-bound nucleic acid in 6×SSC at about 45° C. followed by one or more washes in 0.1×SSC/0.2% SDS at about 68° C., or under other hybridization conditions which are apparent to those of skill in the art (see, for example, Ausubel, F. M. et al., eds., 1989, Current Protocols in Molecular Biology, Vol. 1, Green Publishing Associates, Inc. and John Wiley & Sons, Inc., New York, at pp. 6.3.1-6.3.6 and 2.10.3). Preferably, the polynucleotides that hybridize to the complements of the DNA sequences disclosed herein encode gene products, e.g., gene products that are functionally equivalent to a gene product encoded by one of the enzyme genes or fragments thereof.

As described above, enzyme gene sequences include not only degenerate nucleotide sequences that encode the amino acid sequences of SEQ ID NO: 3, 6, 9, 12, 15, 18, 21, 24, 27, 30, 33, 36, 39, 42, 45, 48, 51, 54, 57, 60, 63, 66, 69, and 72, but also degenerate nucleotide sequences that when translated in organisms other than Aspergillus fumigatus, would yield a polypeptide comprising one of the amino acid sequences of SEQ ID NO: 3, 6, 9, 12, 15, 18, 21, 24, 27, 30, 33, 36, 39, 42, 45, 48, 51, 54, 57, 60, 63, 66, 69, and 72, or a fragment thereof. One of skill in the art would know how to select the appropriate codons or modify the nucleotide sequences of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67 and 70, when using the enzyme gene sequences in A. fumigatus or in other organisms. For example, in Candida albicans, the codon CTG encodes a serine residue instead of leucine residue.

Moreover, the term “enzyme gene” encompasses genes that are naturally occurring in closely related Aspergillus species or variant strains of A. fumigatus, that share extensive nucleotide sequence homology with A. fumigatus genes having one of the DNA sequences that are set forth in SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70. It is contemplated that methods for identification of the enzyme genes of A. fumigatus can also be applied to orthologs of the same genes in A. fumigatus and other fungal species, including but not limited to other Aspergillus species.

In another embodiment, the invention also encompasses the following polynucleotides, host cells expressing such polynucleotides and the expression products of such nucleotides: (a) polynucleotides that encode portions of enzyme gene product that corresponds to its active sites and/or functional domains, and the polypeptide products encoded by such nucleotide sequences, and in which, in the case of secreted gene products, such domains include, but are not limited to signal sequences; and (b) polynucleotides that encode fusion proteins containing an enzyme gene product or one of its active sites and/or domains fused to another polypeptide.

The invention also includes polynucleotides, preferably DNA molecules, that hybridize to, and are therefore the complements of, the DNA sequences of the enzyme gene sequences. Such hybridization conditions can be highly stringent or less highly stringent, as described above and known in the art. The nucleic acid molecules of the invention that hybridize to the above described DNA sequences include oligodeoxynucleotides (“oligos”) which hybridize to the enzyme gene under highly stringent or stringent conditions. In general, for oligos between 14 and 70 nucleotides in length the melting temperature (Tm) is calculated using the formula:

Tm(° C.)=81.5+16.6(log[monovalent cations (molar)]+0.41(% G+C)−(500/N)

where N is the length of the probe. If the hybridization is carried out in a solution containing formamide, the melting temperature may be calculated using the equation:

Tm(° C.)=81.5+16.6(log[monovalent cations (molar)])+0.41(% G+C)−(0.61)(% formamide)−(500/N).

where N is the length of the probe. In general, hybridization is carried out at about 20-25 degrees below Tm (for DNA-DNA hybrids) or about 10-15 degrees below Tm (for RNA-DNA hybrids). Other exemplary highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos).

In another embodiment of the invention, RNA capable of encoding enzyme gene protein sequences are provided. Such RNA molecules can be chemically synthesized using, for example, synthesizers. See, for example, the techniques described in Oligonucleotide Synthesis, 1984, Gait, M. J. ed., IRL Press, Oxford, which is incorporated by reference herein in its entirety. Alternatively, the RNA molecules can be generated biologically by transcription of one of the DNA molecules described above.

In various embodiments, these nucleic acid molecules, DNA or RNA, can encode or act as enzyme gene antisense molecules, useful, for example, in enzyme gene regulation and/or as antisense primers in amplification reactions of enzyme gene nucleotide sequences. Further, such sequences can be used as part of ribozyme and/or triple helix sequences, also useful for enzyme gene regulation. Still further, such molecules can be used as components of diagnostic methods whereby the presence of the fungus can be detected. The uses of these nucleic acid molecules are discussed in detail below.

Fragments of the enzyme genes of the invention can be at least 16 nucleotides in length. In alternative embodiments, the fragments can be at least about 20, 30, 40, 50, 60, 70, 80, 90, 100, 200, 300, 400, 500, 1000, 1500, 2000, 2500, 3000, 3500, 4000, 4500, 5000 or more contiguous nucleotides in length. Alternatively, the fragments can comprise nucleotide sequences that encode about 10, 20, 25, 30, 35, 40, 45, 50, 55, 60, 65, 70, 75, 80, 85, 90, 100, 150, 200, 250, 300, 350, 400, 450, 500, 600, 700, 800, 900, 1000 or more contiguous amino acid residues of the target gene products. Fragments of the enzyme genes of the invention can also refer to exons or introns of the above described nucleic acid molecules, as well as portions of the coding regions of such nucleic acid molecules that encode functional domains such as signal sequences and active site(s). Many such fragments can be used as nucleic acid probes for the identification of other homologous genes of A. fumigatus in the same enzyme family.

In another embodiment, the present invention is directed toward the regulatory regions that are found upstream and downstream of the coding sequences disclosed herein, which are readily determined and isolated from the genomic sequences provided herein. Included within such regulatory regions are, inter alia, promoter sequences, upstream activator sequences, as well as binding sites for regulatory proteins that modulate the expression of the genes identified herein.

The nucleotide sequences of enzyme genes of Aspergillus fumigatus can be used to produce recombinant enzymes, and fragments thereof. The recombinantly produced polypeptide and fragments thereof can be used individually, or in combination as an immunogen or a subunit vaccine to elicit a protective immune response in animals or subjects at high risk of developing a clinical condition, such as those that are under continual exposure of high levels of Aspergillus fumigatus conidia.

The nucleotide sequences of the invention can be used as genetic markers and/or sequence markers to aid the development of a genetic, physical, or sequence map of the Aspergillus fumigatus genome. The nucleotide sequences and corresponding gene products of the invention can also be used to detect the presence of A. fumigatus. Hybridization and antibody-based methods well known in the art can be used to determine the presence and concentration of the nucleotide sequences and corresponding gene products of the invention.

The nucleotide sequences can also be used for identifying inhibitors of the enzymes which may have therapeutic effects, given the fact that the enzymes may play a role in the invasion of a host during an infection.

5.2.2. Homologous Enzyme Genes

In another embodiment, in addition to the nucleotide sequences of Aspergillus fumigatus described above, homologs or orthologs of the enzyme genes of the invention as can be present in A. fumigatus and other fungal species are also encompassed. Particularly preferred are homologs or orthologs in filamentous fungi and yeasts. These enzyme genes can be identified and isolated by molecular biological techniques well known in the art.

“Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (i.e., Penicillium), Emericella and Eurotium (i.e., Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Altemaria. Representative groups of Zygomycota include, e.g., Rhizopus and Mucor.

“Filamentous fungi” include all filamentous forms of the subdivision Eumycota and Oomycota (as defined by Hawksworth et al., 1995, supra). The filamentous fingi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

“Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). For the purposes of this invention, yeast shall be defined as described in Biology and Activities of Yeast (Skinner, F. A., Passmore, S. M., ana Davenport, R. R., eds, Soc. App. Bacteriol. Symposium Series No. 9, 1980). The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathem et al., editors, 1981).

Accordingly, the present invention provides fungal nucleotide sequences that are hybridizable to the polynucleotides of the enzyme genes. In one embodiment, the present invention encompasses an isolated nucleic acid comprising a nucleotide sequence that is at least 50% identical to a nucleotide sequence selected from the group consisting of SEQ ID NO.: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70.

In another embodiment, the present invention encompasses an isolated nucleic acid comprising a fungal nucleotide sequence that hybridizes under medium stringency conditions to a second nucleic acid that consists of a nucleotide sequence selected from the group consisting of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70.

In yet another embodiment, the present invention includes an isolated nucleic acid comprising a fungal nucleotide sequence that encodes a polypeptide the amino acid sequence of which is at least 50% identical to an amino acid sequence selected from the group consisting of SEQ ID No. 3, 6, 9, 12, 15, 18, 21, 24, 27, 30, 33, 36, 39, 42, 45, 48, 51, 54, 57, 60, 63, 66, 69, and 72.

The nucleotide sequences of the invention still further include fungal nucleotide sequences that have at least 40%,45%, 50%, 55%, 60%, 65%, 70%, 75%, 80%, 85%, 90%, 95%, 96%, 97%, 98%, 99% or more nucleotide sequence identity to the nucleotide sequences set forth in SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, 70, 73, 76, 79.

The nucleotide sequences of the invention also include fungal nucleotide sequences that encode polypeptides having at least 25%, 30%, 35%, 40%, 45%, 50%, 55%, 60%, 65%,70%, 75%, 80%, 85%, 90%, 95%, 96%,97%, 98%, 99% or higher amino acid sequence identity or similarity to the amino acid sequences set forth in SEQ ID NO: 3, 6, 9, 12, 15, 18, 21, 24, 27, 30, 33, 36, 39, 42, 45, 48, 51, 54, 57, 60, 63, 66, 69, and 72.

To determine the percent identity of two amino acid sequences or of two nucleotide sequences, the sequences are aligned for optimal comparison purposes (e.g., gaps can be introduced in the sequence of a first amino acid or nucleotide sequence for optimal alignment with a second amino acid or nucleotide sequence). See, for example, the method of Huang and Miller (1991, Adv. Appl. Math, 12:373-381). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100). In one embodiment, the two sequences are substantially similar in length.

The determination of percent identity between two sequences can also be accomplished using a mathematical algorithm. A preferred, non-limiting example of a mathematical algorithm utilized for the comparison of two sequences is the algorithm of Karlin and Altschul (1990) Proc. Natl. Acad. Sci. U.S.A. 87:2264-2268, modified as in Karlin and Altschul (1993) Proc. Natl. Acad. Sci. U.S.A. 90:5873-5877. Such an algorithm is incorporated into the NBLAST and XBLAST programs of Altschul et al., 1990, J. Mol. Biol. 215:403-0. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Although the nucleotide sequences and amino acid sequences of homologs or orthologs of many enzyme genes in Saccharomyces cerevisiae is published, as well as those homologs or orthologs of enzyme genes in Candida albicans which may be available as database version 6 assembled by the Candida albicans Sequencing Project and is accessible by internet at the web sites of Stanford University and University of Minnesota (See http://www-sequence.stanford.edu:8080/and http://alces.med.umn.edu/Candida.html), uses of many of such genes in S. cerevisiae or in C. albicans as industrial enzymes are not known and are thus specifically provided by the invention.

In addition, the genuses of isolated nucleic acid molecules provided in various embodiments of the invention does not comprise the nucleotide sequence of Genbank Accession No. D63338 encoding a tannase of Aspergillus oryzae, Genbank Accession No. AB022429 encoding a cellobiohydrolase II of Acremonium celluloticus Y-94, Genbank Accession No. AE004826 encoding an enzyme of Pseudomonas aeruginosa, Genbank Accession No. U56240 encoding a glucose oxidase of Talaromyces flavus, Genbank Accession No. AF012277 encoding a glucose oxidase of Penicillium amagasakiense, Genbank Accession No. U59804 encoding a phytase of Aspergillus fumigatus, Genbank Accession No. S37150 encoding a beta-galactosidase of Aspergillus niger, Genbank Accession No. A00968 encoding a beta-galactosidase of Aspergillus niger, Genbank Accession No. AJ304803 encoding a glucoamylase of Talaromyces emersonii, Genbank Accession No. AJ289046 encoding a fructosyltransferase of Aspergillus sydowii, Genbank Accession No. A84689 encoding a protein product of Aspergillus tubingensis, Genbank Accession No. X12726 encoding an alpha-pre-amylase of Aspergillus oryzae, Genbank Accession No. AB008370 encoding an acid-stable alpha-amylase of Aspergillus kawachii, Genbank Accession No. AF208225 encoding an alpha-anylase Amy A of Aspergillus nidulans, Genbank Accession No. AF104823 of a gene product of Aspergillus fumigatus, Genbank Accession No. 010460 encoding a glucoarnylase Aspergillus shirousami, Genbank Accession No. AF052061 encoding a polygalacturonase of Ophiostoma novo-ulmi, Genbank Accession No. X58892 encoding a polygalacturonase of Aspergillus niger, Genbank Accession No. Y18805 encoding an endo-polygalacturonase B of Aspergillus niger, Genbank Accession No. AB003085 encoding XynG1 of Aspergillus oryzae, Genbank Accession No. AB044941 encoding a xylanase G2 of Aspergillus oryzae, and Genbank Accession No. AB035540 encoding a xylanase A of Penicillium sp.40. The gene products encoded by the foregoing Genbank nucleotide sequences are also not included in the genuses of gene products contemplated in the present invention.

To isolate homologous enzyme genes, the Aspergillus fumigatus enzyme gene sequence described above can be labeled and used to screen a cDNA library constructed from mRNA obtained from the organism of interest, including but not limited to A. fumigatus. Accordingly, nucleic acid probes, preferably detectably labeled, consisting of any one of the nucleotide sequences of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70 are encompassed. Hybridization conditions should be of a lower stringency when the cDNA library was derived from an organism different from the type of organism from which the labeled sequence was derived. cDNA screening can also identify clones derived from alternatively spliced transcripts in the same or different species. Alternatively, the labeled probe can be used to screen a genomic library derived from the organism of interest, again, using appropriately stringent conditions. Low stringency conditions will be well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Spring Harbor Press, N.Y.; and Ausubel et al., 1989, Current Protocols in Molecular Biology, (Green Publishing Associates and Wiley Interscience, N.Y.).

Further, a homologous enzyme gene sequence can be isolated by performing a polymerase chain reaction (PCR) using two degenerate oligonucleotide primer pools designed on the basis of amino acid sequences within the enzyme gene of interest. The template for the reaction can be cDNA obtained by reverse transcription of mRNA prepared from the organism of interest. The PCR product can be subcloned and sequenced to ensure that the amplified sequences represent the sequences of a homologous enzyme gene sequence.

The PCR fragment can then be used to isolate a full length cDNA clone by a variety of methods well known to those of ordinary skill in the art. Alternatively, the labeled fragment can be used to screen a genomic library.

PCR technology can also be utilized to isolate full length cDNA sequences.

For example, RNA can be isolated, following standard procedures, from an organism of interest. A reverse transcription reaction can be performed on the RNA using an oligonucleotide primer specific for the most 5′ end of the amplified fragment for the priming of first strand synthesis. The resulting RNA/DNA hybrid can then be “tailed” with guanines using a standard terminal transferase reaction, the hybrid can be digested with RNAase H, and second strand synthesis can then be primed with a poly-C primer. Thus, cDNA sequences upstream of the amplified fragment can easily be isolated. For a review of cloning strategies which can be used, see e.g., Sambrook et al., 1989, Molecular Cloning, A Laboratory Manual, Cold Springs Harbor Press, N.Y.; and Ausubel et al., 1989, Current Protocols in Molecular Biology, (Green Publishing Associates and Wiley Interscience, N.Y.).

Additionally, an expression library can be constructed utilizing DNA isolated from or cDNA synthesized from the organism of interest. In this manner, gene products made by the homologous enzyme gene can be expressed and screened using standard antibody screening techniques in conjunction with antibodies raised against the Aspergillus fumigatus gene product, as described, below. (For screening techniques, see, for example, Harlow, E. and Lane, eds., 1988, “Antibodies: A Laboratory Manual,” Cold Spring Harbor Press, Cold Spring Harbor). Library clones detected via their reaction with such labeled antibodies can be purified and subjected to sequence analysis by well known methods.

Alternatively, a database may be searched to determine whether any amino acid sequences or nucleotide sequences display a certain level of homology or sequence identity with respect to the enzyme genes or enzymes. A variety of such databases are available to those skilled in the art, including GenBank and GenSeq. In various embodiments, the databases are screened to identify nucleic acids with at least 97%, at least 95%, at least 90%, at least 85%, at least 80%, at least 70%, at least 60%, at least 50%, or at least 40% nucleotide sequence identity to an enzyme gene sequence, or a portion thereof. In other embodiments, the databases are screened to identify polypeptides having at least 99%, at least 95%, at least 90%, at least 85%, at least 80%, at least 70%, at least 60%, at least 50%, at least 40% or at least 25% amino acid identity or similarity to a polypeptide encoded by the enzyme genes of the invention.

Alternatively, functionally homologous enzyme sequences or polypeptides may be identified by creating mutations by removing or altering the function of an enzyme gene. Having mutants in the genes of one fungal species offers a method to identify functionally similar genes or related genes (orthologs) in another species, or functionally similar genes in the same species (paralogs), by use of a functional complementation test.

A library of gene or cDNA copies of messenger RNA of genes can be made from a species of interest, and the library cloned into a vector permitting expression of the genes in A. fumigatus. Such a library is referred to as a “heterologous library.” Transformation of the heterologous library into a defined mutant of A. fumigatus that is functionally deficient with respect to the identified enzyme gene, and screening or selecting for a gene in the heterologous library that restores phenotypic function in whole or in part of the mutational defect is said to be “heterologous functional complementation”. In this example, the method permits identification of gene in the species of interest that are functionally related to the mutated gene in A. fumigatus. Inherent in this functional-complementation method, is the ability to restore gene function without the requirement for sequence similarity of nucleic acids or polypeptides; that is, this method permits interspecific identification of genes with conserved biological function, even where sequence similarity comparisons fail to reveal or suggest such conservation.

5.2.3. Mutagenesis of A. fumigatus Enzyme Genes

In another embodiment of the invention, the Aspergillus fumigatus enzyme gene sequences can be used in developing modified or novel enzymes that exhibit particularly desirable chemical and/or physical characteristics. Because of the apparent relatedness of the amino acid sequences among the enzymes of Aspergillus fumigatus and other filamentous fungi, the structure of an enzyme of another fungus can be used to predict the structure of the A. fumigatus enzyme, and aid in the rational modification of the A. fumigatus enzyme for useful and superior properties. The sequences provided by the present invention can also be used as starting materials for the rational modification or design of novel enzymes with characteristics that enable the enzymes to perform better in demanding processes.

In one aspect, the sequence, structural, and functional information of the various members of a single enzyme family of A. fumigatus can be compiled and compared. The invention provides the sequences of three members of each of the following enzyme families: glucose oxidases, xylanases, α-amylases, glucoamylases, and polygalacturonases. The results can be used to generate a structural model for the A. fumigatus enzymes including a determination of the active sites, substrate binding sites, etc. with the aid of computers (Bugg et al., Scientific American, December:92-98 (1993); West et al., TIPS, 16:67-74 (1995); Dunbrack et al., Folding & Design, 2:2742 (1997)). The nucleotide sequences of the enzyme genes can be used to produce recombinantly large amounts of the enzymes sufficient to obtain crystals of the enzymes. Methods known in the art for obtaining crystals and X-ray crystallography can be applied to generate a 3-D structure of an enzyme of the invention. In another aspect, the sequence, structural, and functional information of other homologous enzyme gene sequences can be combined and superimposed to assist in the modeling and design processes. Computer analysis may be performed with one or more of the computer programs including: QUANTA, CHARMM, FlexX, INSIGHT, SYBYL, MACROMODEL and ICM.

In particular, the invention encompasses the uses of nucleotide sequences of the invention to design or to generate modified enzymes which possess temperature optima that are either higher or lower than that of the wild type A. fumigatus enzyme, pH optima that are either higher or lower than that of the wild type A. fumigatus enzyme, specific activities that are higher than that of the wild type A. fumigatus enzyme, or a longer half-life than the wild type A. fumigatus enzyme under a particular process condition, such as the presence of detergents. The enzyme gene nucleotide sequences can be altered by random and site-directed mutagenesis techniques or directed molecular evolution techniques, such as but not limited to the methods described in Arnold (1993, Curr. Opinion Biotechnol. 4:450-455), oligonucleotide-directed mutagenesis (Reidhaar-Olson et al., 1988, Science 241:53-57), chemical mutagenesis (Eckert et al., Mutat. Res. (1987) 178:1-10), site-directed mutagenesis (Kunkel (1985) Proc. Natl. Acad. Sci. USA 82:488492; Oliphant et al., (1986) Gene 44:177-183), error prone PCR (Caldwell and Joyce, 1992, PCR Methods Applic. 2:28-33), cassette mutagenesis (Arkin et al., Proc. Natl. Acad. Sci. USA, 1992, 89:7871-7815), DNA shuffling methods as described in Stemmer et al., 1994, Proc. Natl. Acad. Sci. USA, 91:10747-10751 and in U.S. Pat. Nos. 5,605,793; 6,117,679; and 6,132,970, and the methods as described in U.S. Pat. Nos. 5,939,250, 5,965,408, 6,171,820. In certain embodiments, nucleotide sequences of other related enzyme genes that encodes similar domains, structural motifs, or active sites, or that aligns with a portion of the enzyme gene of the invention with mismatches or imperfect matches, can be used in the mutagenesis process to generate diversity of sequences. It should be understood that for each mutagenesis step in some of the techniques mentioned above, a number of iterative cycles of any or all of the steps may be performed to optimize the diversity of sequences. The above-described methods can be used in combination in any desired order. In many instances, the methods result in a pool of mutant nucleotide sequences or a pool of recombinant host cells comprising mutant nucleotide sequences. The nucleotide sequences or host cells expressing a modified enzyme with the desired characteristics can be identified by screening with one or more enzymatic assays that are well known in the art. The assays maybe carried out under conditions that select for enzymes possessing the desired physical or chemical characteristics. The mutations in the nucleotide sequence can be determined by sequencing the enzyme gene in the clones.

5.2.4. Vectors, Expression Constructs, and Recombinant Host Cells

In another embodiment, the invention also encompasses (a) nucleic acid vectors that comprise a nucleotide sequence comprising any of the foregoing sequences of the enzyme genes and/or their complements (including antisense molecules); (b) expression constructs that comprise a nucleotide sequence comprising any of the foregoing coding sequences of the enzyme genes operably linked with a regulatory element that directs the expression of the coding sequences; and (c) recombinant host cells that comprise any of the foregoing sequences of the enzyme gene, including coding regions operably linked with a regulatory element that directs the expression of the coding sequences in the host cells.

Recombinant DNA methods which are well known to those skilled in the art can be used to construct vectors comprising coding sequences of the enzyme genes, and appropriate transcriptional/translational control signals. The various sequences may be joined in accordance with known techniques, such as restriction, joining complementary restriction sites and ligating, blunt ending by filling in overhangs and blunt ligation, Bal31 treatment, primer repair, in vitro mutagenesis, or the like. Polylinkers and adapters may be employed, when appropriate, and introduced or removed by known techniques to allow for ease of assembly of the DNA vectors and expression constructs. These methods may also include in vivo recombination/genetic recombination. At each stage of the manipulation of the enzyme gene sequences, the fragment(s) may be cloned, analyzed by restriction enzyme, sequencing or hybridization, or the like. A large number of vectors are available for cloning and genetic manipulation. Normally, cloning can be performed in E. coli. See, for example, the techniques described in Sambrook et al., 1989, Molecular Cloning: A Laboratory Manual, Cold Spring Harbor Press, Cold Spring Harbor, N.Y.; Ausubel, 1989, supra; Methods in Enzymology: Guide to Molecular Cloning Techniques, Academic Press, Berger, S. L. and A. R. Kimmel eds., 1987; Pla et al., Yeast 12:1677-1702 (1996); Kinghom and Unkles in Aspergillus, ed. by J. E. Smith, Plenum Press, New York, 1994, Chapter 4, p.65-100.

In various embodiments of the invention, nucleic acid vectors that comprise an enzyme gene sequence of the invention, may further comprise replication functions that enable the transfer, maintenance and propagation of the vectors in one or more species of host cells, including but not limited to E. coli cells, filamentous fungal cells, yeast cells, and Bacillus cells. The choice of the vector will typically depend on the compatibility of the vector with the host cell into which the vector is to be introduced. The vectors may be linear or closed circular plasmids, cosmid, or phagemids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total nucleic acid to be introduced into the genome of the host cell, or a transposon.

A expression construct of the invention comprises a promoter, a nucleotide sequence encoding for an enzyme gene, a transcription termination sequence, and optionally, a selectable marker. If the expression host that is used to produce the polypeptide or peptide does not use the universal genetic code, gene products of the enzyme genes having the amino acid sequences of SEQ ID NO: 3, 6, 9, 12, 15, 18, 21, 24, 27, 30, 33, 36, 39, 42, 45, 48, 51, 54, 57, 60, 63, 66, 69, and 72, may be encoded by nucleotide sequences that conform to the known codon usage in the host. One of skill in the art would know the modifications that are necessary to accommodate for a difference in codon usage. When an expression construct comprising the enzyme gene sequence of the invention is introduced into a host cell by transformation, the enzyme gene sequence is transcribed and translated to produce the corresponding polypeptide, and an increase in the enzyme activity can be demonstrated functionally. Accordingly, the present invention also relates to methods for producing an polypeptide of the present invention comprising (a) cultivating a host cell under conditions conducive to expression of the polypeptide; and (b) recovering the polypeptide.

Any method known in the art for introducing the enzyme gene sequences of the invention into a host cell can be used, including those described hereinbelow. Fungal cells may be transformed by a process involving protoplast formation, transformation of the protoplasts, and regeneration of the cell wall. Suitable procedures for transformation of Aspergillus host cells are described in EP 238 023 and Yelton et al., 1984, Proc. Natl. Acad. Sci. USA 81:1470-1474.

A suitable method of transforming Fusarium species is described by Malardier et al., 1989, Gene 78:147-156. Other methods may include using co-transformation, lithium acetate treatment of conidia, electroporation (Ward et al., 1989, Exp. Mycol. 13:289-293), and microprojectiles (Armaleo et al., 1990, Curr. Genet. 17:97-103). See also Fincham (1989, Microbiol. Rev. 53:148-170), and May (1992, Fungal Technology, in Applied Molecular Genetics of Filamentous Fungi, Blackie Press, Glasgow). Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriol. 153:163; and Hinnen et al., 1978, Proc. Natl. Acad. Sci. USA 75:1920.

The isolation of the enzyme gene sequences of the invention enables the economical production of the respective enzymes on an industrial scale, via the application of techniques known in the art such as gene amplification, the exchange of regulatory elements such as promoters, secretory signals, or combinations thereof. Accordingly, the present invention also comprises an expression host capable of the efficient expression of high levels of peptides or proteins having the enzyme activity of interest and, if desired depending on the application, the expression of additional enzymes as well. Preferably, the enzymes are secreted by the expression host.

For a majority of the industrial applications, the enzymes of the invention are produced by a fungal cell. Preferably, the expression host cell is a filamentous fungal cell which has been used in large scale industrial fermentation. In many instances, the most preferred are host cells that are approved by regulatory authorities, such as the United States Food and Drug Administration, for production of food substances. For example, GRAS (generally-regarded-as-safe) organisms are preferred. In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins. Appropriate cell lines or host systems can be chosen to ensure the correct modification and processing of the foreign protein expressed. Preferably, an expression host is selected which is capable of the efficient secretion of their endogenous proteins. A host cell may also be chosen for deficiencies in extracellular protease activities since the secreted enzyme may be degraded in the culture medium.

Preferred expression hosts include filamentous fungi selected from the genera Acremonium, Aspergillus, Fusarium, Humicola, Myceliophthora, Neurospora, Thielavia, Tolypocladium, Trichodenna, Mucor and Penicillium. In a most preferred embodiment, industrial strains of Aspergillus, especially A. niger, A. ficuum, A. awamori, A. foetidus, A. japonicus and A. oryzae, can be used. Alternatively, Trichodenna reesei, or Mucor miehei, may be used. The fungal host cell can also be a yeast cell of a species of Candida, Kluyveromyces, Saccharomyces, Schizosaccharomyces, Pichia, or Yarrowia. In a preferred embodiment, the yeast host cell is a Saccharomyces cerevisiae, a Saccharomyces carlsbergensis, a Saccharomyces diastaticus, a Saccharomyces douglasii, a Saccharomyces kluyveri, a Saccharomyces norbensis, or a Saccharomyces ovifomis cell, a Kluyveromyces lactis cell, or a Yarrowia lipolytica cell.

As used herein, regulatory elements include but are not limited to inducible and non-inducible promoters, enhancers, operators and other elements known to those skilled in the art that enable and regulate expression. Generally, the region 5′ to the open reading frame in the enzyme gene sequence of the invention comprises the transcriptional initiation regulatory region (or promoter) which can be used for expression in fungi. Alternatively, any regulatory region functional in the host may be employed. In preferred embodiments, promoters of genes which are homologous to the enzyme gene sequence to be expressed may be used. Promoters of genes of the expression host are most preferred. For expression in fungal cells, fungal regulatory elements may include those associated with alcohol dehydrogenases (adhA, alcA, alcC; inducible by ethanol), isopenicillin N synthetase (pcbc), pyr4, pyrG, glyceraldehyde-3-phosphate dehydrogenase (gpda, constitutive); mprA (aspartyl protease of Mucor miehel); and promoters isolated from genes involved in carbohydrate metabolism such as amylases (amyA, amy(taka), inducible by starch); glucoamylases (glaA, inducible by maltose, starch, maltodextrin). For further examples, see Van den Hondel et al., Heterologous gene expression infilamentous fungi, chapter 18, pp. 396-428 in More Gene Manipulations in Fungi, Academic Press, 1991. Particularly preferred promoters for use in filamentous fungal host cells are the TAKA amylase, NA2-tpi (a hybrid of the promoters from the genes encoding Aspergillus niger neutral α-amylase and Aspergillus oryzae triose phosphate isomerase), and glaA promoters. In a yeast host, useful promoters are obtained from the Saccharomyces cerevisiae enolase (ENO-1) gene, the S. cerevisiae galactokinase gene (GAL1), the S. cerevisiae alcohol dehydrogenase/glyceraldehyde-3-phosphate dehydrogenase genes (ADH2/GAP), and the S. cerevisiae 3-phosphoglycerate kinase gene, and genes relating to amino acid metabolism (e.g. MET genes) and the acid phosphatase gene. Other useful promoters for yeast host cells include the yeast mating pheromone responsive promoters (e.g. STE2 and STE3), the AOX1 system for Pichia pastoris, the phosphate-responsive promoters (e.g. PH05), and those described by Romanos et al., 1992, Yeast 8:423-488.

In certain instances, specific initiation signals may also be required for efficient translation of inserted enzyme gene coding sequences. These signals include the ATG initiation codon and adjacent sequences. In cases where an entire enzyme gene, including its own initiation codon and adjacent sequences, is inserted into the appropriate expression vector, no additional translational control signals are needed. However, in cases where only a portion of the enzyme gene coding sequence is inserted, or the Aspergillus fumigatus signals are not efficient in a particular host cell, exogenous translational control signals, including, the ATG initiation codon, may be provided. Furthermore, the initiation codon must be in phase with the reading frame of the desired coding sequence to ensure proper translation of the entire sequence. These exogenous translational control signals and initiation codons can be of a variety of origins, both natural and synthetic. For example, (C/T)CA(C/A)(C/A)ATG, may be used with many filamentous fungi (Gurr et al., 1988, in Gene Structure in Eukaryotic Microbes, ed. by Kinghom, Society of General Microbiology Special Publication, 23:93-139, IRL Press, Oxford.

The expression construct of the invention may also comprise a peptide sequence which provides for secretion of the expressed peptide or protein from the host. Various signal sequences (also referred to as leader sequences) may be used. Preferred signal sequences include signal sequences of the homologous enzyme genes of the expression host. The signal peptide coding region may be obtained from a glucoamylase or an amylase gene from an Aspergillus species, a lipase or proteinase gene from a Rhizomucor species, the gene for the α-factor from Saccharomyces cerevisiae, an amylase or a protease gene from a Bacillus species, or the calf preprochymosin gene. However, any signal peptide coding region capable of directing the expressed enzyme into the secretory pathway of a host cell of choice may be used in the present invention. The nucleotide sequence encoding the signal sequence maybe joined directly through the sequence encoding the processing signal to the sequence encoding the desired protein, or through a short linker, usually fewer than ten codons. The short linker may also contain a protease cleavage site, such as but not limited to the Kex2 or factor Xa cleavage sites.

A transcriptional termination regulatory region is operably linked to the 3′ terminus of the nucleic acid sequence encoding the polypeptide. A polyadenylation sequence may also be included in this region. Any terminator which is functional in the host cell of choice may be used in the present invention. The terminator sequence may be from any gene of Aspergillus fumigatus including but not limited to those of the enzyme genes of the invention, the homologous enzyme gene of the expression host, or any other termination sequence known in the art. Preferred terminators for filamentous fungal host cells are obtained from the genes encoding A. oryzae TAKA amylase, A. niger glucoarnylase, A. nidulans anthranilate synthase, A. niger alpha-glucosidase, and Fusarium oxysporum trypsin-like protease. Preferred terminators for yeast host cells are obtained from the genes encoding Saccharomyces cerevisiae enolase, S. cerevisiae cytochrome C (CYC1), or S. cerevisiae glyceraldehyde-3-phosphate dehydrogenase. Useful polyadenylation sequences for yeast host cells are described by Guo and Sherman, 1995, Molecular Cellular Biology 15:5983-5990.

For long-term, high-yield production of recombinant proteins, stable expression is preferred. For example, cell lines which stably express the enzyme gene protein can be engineered. Host cells can be transformed with nucleic acid controlled by appropriate expression control elements and a selectable marker. Following the introduction of the foreign nucleic acid, engineered cells can be allowed to grow for 1-2 days in an enriched medium, and then are switched to a selective medium. The selectable marker in the recombinant plasmid confers resistance to the selection after the plasmid integrates into the chromosome via a double cross-over event. Such cells form foci when cultured under selection, which in turn can be cloned and expanded into cell lines. In general, transformants with multiple integrated copies of the expression construct can be obtained by selection and/or amplification, and are preferred since a higher copy number usually results in higher protein production. Alternatively, if an autologously replicating vector is used, the vector can be maintained extrachromosomally in the cells.

As the vectors of the present invention may be integrated into the host cell genome when introduced into a host cell, the vector may rely on the nucleotide sequence encoding the polypeptide or any other element of the vector for stable integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location in a chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be nucleic acids comprising non-encoding or encoding nucleotide sequences. On the other hand, the vector may be integrated into the genome of the host cell by non-homologous recombination. These nucleic acids may comprise sequence that is homologous with a target sequence in the genome of the host cell, and, furthermore, may be non-encoding or encoding sequences.

In one particular embodiment, the enzyme gene of the invention replaces the homolgous enzyme gene of the expression host. The replacement can be effected by any techniques, including homologous recombination. The enzyme gene of the invention can be expressed by regulatory elements associated with the homologous gene in the chromosome or by heterologous regulatory elements. One advantage of this approach is the likelihood that expression of the enzyme gene will be similar to that of the homologous gene. Another advantage of such an expression host is simplification of purification of the desired enzyme, since the native homolgous enzyme is not produced.

A selection or selectable marker may or may not be part of the nucleic acid vector comprising the enzyme gene sequence. Typically, a selectable marker is a gene the product of which provides for drug or viral resistance, resistance to heavy metals, prototrophy to auxotrophs, and the like. The selection marker will have its own regulatory regions to allow for independent expression of the marker. A large number of transcriptional regulatory regions, preferably regions from genes that are under constituitive expression, are known and may be used in conjunction with the marker gene. Since the recombinant enzyme gene sequences of the invention are preferably introduced into a host that can be used for industrial production, selection markers to monitor the transformation are preferably dominant selection markers, i.e., no mutations have to be introduced into the host strain to be able to use these selection markers. Examples of dominant selectable markers that confer resistance to antibiotics include but are not limited to the ble gene that confers resistance to phleomycin (Austin et al., Gene 1990, 93:157-162), the hph gene that confers resistance to hygromycin B (Tang et al., 1992, Mol. Microbiol., 6:1663-1671), the benA gene that confers resistance to Benomyl (Seip et al., 1990, Appl. Environ. Microbiol. 56:3686-3692); the oligomycin-resistant ATP synthase subunit gene (oliC, Ward et al., 1988, Curr. Genet. 14:37); the bar gene (phosphinothricin acetyltransferase) and the gene that confers glufosinate resistance. Examples of bacterial selectable markers are the dal genes from Bacillus subtilis or Bacillus licheniformis, or markers which confer antibiotic resistance such as ampicillin, kanamycin, chloramphenicol or tetracycline resistance. Where antibiotic resistance is employed, the concentration of the antibiotic for selection will vary depending upon the antibiotic, generally ranging from about 30 to 300 g/ml of the antibiotic.

The other type of selection markers are nutritional markers that are used for complementation in specific types of mutant cells. For example, transformation of A. nidulans has been demonstrated by using plasmids containing the Neurospora crassa pyr-4 gene (Ballance, D. J. et al., Biochem. Biophys. Res. Commun., 112 (1983):284-289), the A. nidulans amdS gene (Tilburn, J. G. et al., Gene 26 (1983):205-221), the A. nidulans trpC gene (anthranilate synthase; Yelton, M. M. et al., Proc. Natl. Acad. Sci. U.S.A., 81 (1984):1470-1474) and the A. nidulans argb gene (John, M. A. and Peberdy J., Microb. Technol. 6 (1984):386-389). Transformation of Aspergillus niger with the amdS gene of A. nidulans was also described (Kelly, J. M. and Hynes, M. J., EMBO Journal 4 (1985), 475479) and amdS was shown to be a selection marker for use in transformation of A. niger that cannot grow strongly on acetamide as a sole nitrogen source. Transformation of A. niger using the argb gene of A. nidulans has also been described (Buxton, F. P. et al., Gene 37 (1985),207-214). Other examples of nutritional markers may include but are not limited to sC (sulfate adenyltransferase), nitrate utilization (niaD, Unkles et al., 1989, Gene 78:157-166); quinic acid utilization (qutE; Streatfield et al., 1992, Mol. Gen. Genet 233:231-240), and pyrG which complements a orotidine-5′-phosphate decarboxylase mutant (Weidner et al., Curr Genet 1998 May;33(5):378-85). Preferred for use in an Aspergillus cell are the amdS and pyrG markers of A. nidulans or A. oryzae and the bar marker of Streptomyces hygroscopicus. Suitable markers for yeast host cells are ADE2, HIS3, LEU2, LYS2, MET3, TRP1, URA3, and NST (nouseothricin resistance).

The expression host cells or transformants of the invention maybe cultured in any nutrient medium suitable for growth and expression of proteins. Low concentrations of a protease inhibitor may be employed (if the enzyme to be produced is not a protease), such as phenylmethylsulfonyl fluoride, leupeptin, α2-macroglobulins, pepstatin, or the like. Usually, the concentration will be in the range of about 1 μg/ml to 1 mg/ml. However, in some instances, the protease gene(s) of the expression host may be inactivated in order to avoid or reduce degradation of the desired protein. For example, the host cell may be cultivated by shake flask cultivation, small-scale or large-scale fermentation (including continuous, batch, fed-batch, or solid state fermentations) in laboratory or industrial fermentors performed under conditions allowing the polypeptide to be expressed and/or isolated. The cultivation takes place in a suitable nutrient medium comprising carbon and nitrogen sources and inorganic salts, using procedures known in the art (see, e.g., references for bacteria and yeast; Bennett, J. W. and LaSure, L., editors, More Gene Manipulations in Fungi, Academic Press, CA, 1991). Suitable media are available from commercial suppliers or may be prepared according to published compositions (e.g., in catalogues of the American Type Culture Collection). For example, for a fungal fermentation, spores and subsequently cells are transferred through a series of batch fermentations in Erlenmeyer flasks to a 10 liter fermentor. After growth in batch culture, the contents of the 10 liter fermentor are used as inoculum for a final 500 liter batch fermentation.

If the polypeptide is secreted into the nutrient medium, the polypeptide can be recovered directly from the medium. If the polypeptide is not secreted, it is recovered from cell lysates. The polypeptides may be detected using methods known in the art that are specific for the polypeptides. These detection methods may include use of specific antibodies, formation of an enzyme product, or disappearance of an enzyme substrate. An enzyme assay may be used to determine the activity of the polypeptide. Various methods for concentrating, and purifying the product if necessary, may be employed, such as filtration, centrifugation, solvent-solvent extraction, combinations thereof, or the like.

Protein purification techniques are well known in the art. Chromatographic methods such as ion-exchange chromatography, gel filtration, use of hydroxyapaptite columns, immobilized reactive dyes, chromatofocusing, and use of high-performance liquid chromatography (HPLC), maybe used to purify the protein. Electrophoretic methods such as one-dimensional gel electrophoresis, high-resolution two-dimensional polyacrylamide electrophoresis, isoelectric focusing, and others are also contemplated as purification methods. Also, affinity chromatographic methods, comprising solid phase bound-antibody, ligand presenting columns and other affinity chromatographic matrices are contemplated as purification methods in the present invention. Alternatively, epitope tagging of the protein can be used to allow simple one step purification of the protein.

A variety of non-fungal host-expression vector systems can also be utilized to express the enzyme gene coding sequences of the invention. Such host-expression systems represent vehicles by which the coding sequences of interest can be produced and subsequently purified, but also represent cells which can, when transformed or transfected with the appropriate nucleotide coding sequences, produce the enzyme gene protein of the invention in situ. These include but are not limited to microorganisms such as bacteria (e.g., E. coli, B. subtilis) transformed with recombinant bacteriophage DNA, plasmid DNA or cosmid DNA expression vectors containing enzyme gene protein coding sequences; insect cell systems infected with recombinant virus expression vectors (e.g., baculovirus) containing the enzyme gene protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing enzyme gene protein coding sequences; or mammalian cell systems (e.g. COS, CHO, BHK, 293, 3T3) harboring recombinant expression constructs containing promoters derived from the genome of mammalian cells (e.g., metallothionein promoter) or from mammalian viruses (e.g. the adenovirus late promoter; the vaccinia virus 7.5K promoter).

In bacterial systems, a number of expression vectors can be advantageously selected depending upon the use intended for the enzyme gene protein being expressed. For example, when a large quantity of such a protein is to be produced, for the generation of antibodies or to screen peptide libraries, for example, vectors which direct the expression of high levels of fusion protein products that are readily purified can be desirable. Such vectors include, but are not limited, to the E. coli expression vector pUR278 Luther et al., 1983, EMBO J. 2:1791), in which the enzyme gene protein coding sequence can be ligated individually into the vector in frame with the lacZ coding region so that a fusion protein is produced; pIN vectors (Inouye & Inouye, 1985, Nucleic Acids Res. 13:3101-3109; Van Heeke & Schuster, 1989, J. Biol. Chem. 264:5503-5509); and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned enzyme gene protein can be released from the GST moiety. For expression in bacteria, useful regulatory elements include but are not limited to the lac system, the trp system, the tet system and other antibiotic-based repression systems (e.g. PIP), the TAC system (DeBoer et al., 1983, Proc. Natl. Acad. Sci. USA 80:21-25), the TRC system, the major operator and promoter regions of phage A, and the control regions of fd coat protein. Other examples of useful promoters may include that of the Streptomyces coelicolor agarase gene (dagA), the Bacillus subtilis levansucrase gene (sacB), the Bacillus licheniformis alpha-amylase gene (amyL), the Bacillus stearothennophilus maltogenic amylase gene (amyM), the Bacillus amyloliquefaciens alphα-amylase gene (amyQ), the Bacillus licheniformis penicillinase gene (penP), and the Bacillus subtilis xylA and xylB genes.

The choice of a bacterial host cell will to a large extent depend upon the enzyme gene and its application. The host cell may be a bacteria that have been used for producing industrial enzymes. Useful host cells are bacterial cells such as gram positive bacteria including, but not limited to, a Bacillus cell, e.g., Bacillus subtilis, Bacillus licheniformis, Bacillus lentus, Bacillus brevis, Bacillus stearothernophilus, Bacillus alkalophilus, Bacillus amyloliquefaciens, Bacillus coagulans, Bacillus circulans, Bacillus lautus, Bacillus megaterium, and Bacillus thuringiensis; or a Streptomyces cell, e.g., Streptomyces lividans or Streptomyces murinus. In a preferred embodiment, the bacterial host cell is a Bacillus lentus, a Bacillus licheniformis, a Bacillus subtilis, or a Bacillus stearothermophilus cell. The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), orby conjugation (see, e.g., Koehler and Thome, 1987, Journal of Bacteriology 169:5771-5278).

5.3. Aspergillus fumigatus Gene Products

The enzyme gene products encompassed in the present invention include those gene products (e.g., RNA or proteins) that are encoded by the enzyme gene sequences as described above, such as, the enzyme gene sequences set forth in SEQ ID NO: 2, 5, 8, 11, 14, 17, 20, 23, 26, 29, 32, 35, 38, 41, 44, 47, 50, 53, 56, 59, 62, 65, 68, and 71. The enzyme gene products of the invention also encompasses those RNA or proteins that are encoded by the the genomic sequences of the enzyme genes as set forth in SEQ ID NO 1, 4, 7, 10, 13, 16, 19, 22, 25, 28, 31, 34, 37, 40, 43, 46, 49, 52, 55, 58, 61, 64, 67, and 70. The enzymes of the invention comprises an amino acid sequence selected from the group consisting of SEQ ID NO. 3, 6, 9, 12, 15, 18, 21, 24, 27, 30, 33, 36, 39, 42, 45, 48, 51, 54, 57, 60, 63, 66, 69, and 72.

The enzymes of the invention display at least one of the chemical characteristics or activity of an enzyme selected from the group consisting of tannase, cellulase, glucose oxidase, glucoamylase, α-amylase, phytase, β-galactosidase, sucrase, lipase, laccase, xylanase and polygalacturonase. As used herein, the term “chemical characteristic” of an enzyme of the invention refers to the substrate or chemical functionality upon which the enzyme acts and/or the catalytic reaction performed by the enzyme; e.g., the catalytic reaction may be hydrolysis (hydrolases) and the chemical functionality may be the type of bond upon which the enzyme acts (esterases cleave ester bonds) or may be the particular type of structure upon which the enzyme acts (a glycosidase which acts on glycosidic bonds).

As used herein, a “physical characteristic” with respect to an enzyme means a property (other than a chemical characteristic), such as optimum pH for catalytic reaction; temperature stability; optimum temperature for catalytic reaction; organic solvent tolerance; metal ion selectivity, detergent tolerance, etc. The enzymes of the invention can catalyzes their respective enzymatic reaction at a range of temperatures from ambient temperature to elevated temperature, for example, room temperature, i.e., 20° to 25° C., body temperature, i.e, about 37° C., and higher temperatures such as 45° C., 50° C., 55° C., 60° C. and up to 70° C. Since A. fumigatus is a thermophilic fungus, the enzymes of this organism are expected to be stable at 70° C., and even at higher temperature up to 100° C. See Latge, 1999, Clin. Microbiol. Rev. 12:210-350 and Pasarnontes et al., 1997 Applied Environ. Microbiol. 63:1696-1700. The spores of A. fumigatus are also known to survive at extreme low temperature. Accordingly, the enzymes of the invention are also expected to display enzymatic activity and/or stability at low temperatures, e.g., below 10° C., 4° C., −20° C., and −80° C. The enzymes of the invention also display increased half-life in storage and increased organic solvent tolerance.

The enzyme gene products of the invention can be readily produced, e.g., by synthetic techniques or by methods of recombinant DNA technology using techniques that are well known in the art. In one embodiment, the polypeptides and peptides of the invention can be synthesized or prepared by techniques well known in the art. See, for example, Creighton, 1983, Proteins: Structures and Molecular Principles, W. H. Freeman and Co., N.Y.

In addition, the methods and compositions of the invention also encompass proteins and polypeptides that represent functionally equivalent gene products. Such functionally equivalent gene products include, but are not limited to, natural variants of the polypeptides having an amino acid sequence set forth in SEQ ID NO: 3, 6, 9, 12, 15, 18, 21, 24, 27, 30, 33, 36, 39, 42, 45, 48, 51, 54, 57, 60, 63, 66, 69, and 72.

Such equivalent enzyme gene products can contain, e.g. deletions, additions or substitutions of amino acid residues within the amino acid sequences encoded by the enzyme gene sequences described above, but which result in a silent change, thus producing a functionally equivalent product. Amino acid substitutions can be made on the basis of similarity in polarity, charge, solubility, hydrophobicity, hydrophilicity and/or the amphipathic nature of the residues involved. For example, nonpolar (i.e., hydrophobic) amino acid residues can include alanine (Ala or A), leucine (Leu or L), isoleucine (Ile or I), valine (Val or V), proline (Pro or P), phenylalanine (Phe or F), tryptophan (Trp or W) and methionine (Met or M); polar neutral amino acid residues can include glycine (Gly or G), serine (Ser or S), threonine (Thr or T), cysteine (Cys or C), tyrosine (Tyr or Y), asparagine (Asn or N) and glutamine (Gln or Q); positively charged (i.e., basic) amino acid residues can include arginine (Arg or R), lysine (Lys or K) and histidine (His or H); and negatively charged (i.e., acidic) amino acid residues can include aspartic acid (Asp or D) and glutamic acid (Glu or E).

“Functionally equivalent,” as the term is utilized herein, refers to a polypeptide capable of exhibiting a substantially similar enzymatic activity or at least one chemical characteristics as the Aspergillus fumigatus enzyme gene product encoded by one of the enzyme gene sequences described in Table 1. Alternatively, the term “functionally equivalent” can refer to peptides or polypeptides that are capable of interacting with the substrate of an enzyme gene of the invention in a manner substantially similar to the way in which the enzyme gene product would interact with such a substrate. Preferably, the functionally equivalent enzyme gene products of the invention are about the same size and display similar physical characteristics as the enzyme encoded by one of the enzyme gene sequences described in Table 1.

It will be apparent to those skilled in the art that such substitutions can be made outside the regions critical to the function of the molecule and still result in an active polypeptide. Amino acid residues essential to the activity of the polypeptide encoded by the isolated nucleic acid sequence of the invention, and therefore preferably not subject to substitution, may be identified according to procedures known in the art, such as site-directed mutagenesis or alanine-scanning mutagenesis (see, e.g., Cunningham and Wells, 1989, Science 244:1081-1085). In the latter technique mutations are introduced at every residue in the molecule, and the resultant mutant molecules are tested for the enzyme activity to identify amino acid residues that are critical to the activity of the molecule. Sites of substrate-enzyme interaction can also be determined by analysis of crystal structure as determined by such techniques as nuclear magnetic resonance analysis, crystallography or photoaffinity labelling (see, e.g., de Vos et al., 1992, Science 255, 306-312; Smith et al., 1992, Journal of Molecular Biology 224:899-904; Wlodaver et al., 1992, FEBS Letters 309, 59-64).

Peptides and polypeptides corresponding to one or more domains of the enzyme gene products (e.g., signal sequences, active sites, or substrate-binding domains), truncated or deleted enzymes (e.g., polypeptides in which one or more domains of a enzyme are deleted) and fusion enzymes (e.g., proteins in which a full length or truncated or deleted enzyme, or a peptide or polypeptide corresponding to one or more domains of an enzyme is fused to an unrelated protein) are also within the scope of the present invention. Such peptides and polypeptides (also referred to as chimeric protein or polypeptides) can be readily designed by those skilled in the art on the basis of the enzyme gene nucleotide and amino acid sequences listed in Table 1. Exemplary fusion proteins can include, but are not limited to, epitope tag-fusion proteins which facilitates isolation of the enzyme gene product by affinity chromatography using reagents that binds the epitope. Other exemplary fusion proteins include fusions to any amino acid sequence that allows, e.g., the fusion protein to be immobilized onto a solid phase, thereby allowing the enzyme to be retained and re-used after a reaction; the fusion protein to be anchored to a cell membrane, thereby allowing the enzyme to be exhibited on a cell surface; or to a luminescent protein which can provide a marker function. Accordingly, the invention provides a fusion protein comprising a fragment of a first polypeptide fused to a second polypeptide, said fragment of the first polypeptide consisting of at least 6 consecutive residues of an amino acid sequence selected from one of SEQ ID NO: 3, 6, 9, 12, 15, 18, 21, 24, 27, 30, 33, 36, 39, 42, 45, 48, 51, 54, 57, 60, 63, 66, 69, and 72.

Other modifications of the enzyme gene product coding sequences described above can be made to generate polypeptides that are better suited, e.g., for expression, for scale up, etc. in a chosen host cell. For example, cysteine residues can be deleted or substituted with another amino acid in order to eliminate disulfide bridges.

The enzyme gene products of the invention preferably comprise at least as many contiguous amino acid residues as are necessary to represent an epitope fragment (that is, for the gene products to be recognized by an antibody directed to the enzyme gene product). For example, such protein fragments or peptides can comprise at least about 8 contiguous amino acid residues from a enzyme gene product. In alternative embodiments, the protein fragments and peptides of the invention can comprise about 6, 8, 10, 20, 25, 30, 35, 40, 45, 50, 55, 60, 65, 70, 75, 80, 85, 90, 95, 100, 150, 200, 250, 300, 350, 400, 450 or more contiguous amino acid residues of a enzyme gene product.

The enzyme gene products used and encompassed in the methods and compositions of the present invention also encompass amino acid sequences encoded by one or more of the above-described enzyme gene sequences of the invention wherein domains often encoded by one or more exons of those sequences, or fragments thereof, have been deleted. The enzyme gene products of the invention can still further comprise post translational modifications, including, but not limited to, glycosylations, acetylations and myristylations.

Depending on the industrial application, the enzyme gene protein can be labeled, either directly or indirectly, to facilitate its detection. Any of a variety of suitable labeling systems can be used including but not limited to radioisotopes such as ¹²⁵I; enzyme labeling systems that generate a detectable colorimetric signal or light when exposed to substrate; and fluorescent labels. Indirect labeling involves the use of a protein, such as a labeled antibody, which specifically binds to either a enzyme gene product. Such antibodies include but are not limited to polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

Enzymes of the invention can be used on an industrial scale as catalysts for processing various crude or raw materials. The invention encompasses enzymatic compositions comprising a catalytically effective amount of an enzyme of the invention isolated, purified or enriched to various degrees, e.g., the enzyme can constitute about 0.1%, 0.25%, 0.5%, 0.75%, 1%, 2%, 5%, 10%, 20%, 40%, 50%, 75%, 80%, 90%, 95%, 99% of the total protein in the composition. The enzymatic compositions are in a form suitable for use in the intended industrial processes, and may contain additional enzymes, stabilizing agents, preservatives, protease inhibitors, detergents, antifoaming agents, etc. Often these processes are cost-effective only when the enzymes can be re-used many times. For reuse of the enzymes, the enzymes need to be separated from the bulk of the process. This can be achieved when the enzymes are attached to a carrier or solid phase which can be isolated, for example by draining, filtration or centrifugation. This can also be achieved if the substrate is flowed across the surface of the solid phase where contacts with the enzymes are made. Accordingly, the present invention encompasses enzymes of the invention which exist not only in free-flowing soluble form, but also in immobilized or solid forms.

In one embodiment, the enzymes of the invention can be stabilized by their association with cell membranes, or whole microbial cells, viable or non-viable. Cells can be further stabilized by entrapment in various kinds of gel or attached to the surface of solid particles. Alternatively, the cells are homogenized and cross-linked with glutaraldehyde to form an insoluble yet permeable matrix. Accordingly, the invention encompasses immobilized cell compositions or cell lysate compositions comprising an enzyme of the invention.

In another embodiment, the enzymes of the invention are immobilized in the form of proteins purified to varying degrees as described above. Any known method for immobilization of enzyme based on chemical and physical binding of the enzyme to a soild phase, e.g, polysaccharides, glass, synthetic polymers, magnetic particles, which are usually modified with functional groups, such as amine, carboxy, epoxy, phenyl or alkane to enable covalent coupling to amino acid side chains on the enzyme surface, can be used. The solid phase can be porous, with pore diameters in the range of 30 to 300 nm. Ionic and non-ionic adsorption to porous support can be a simple and effective method of immobilization. The enzymes can also be entrapped or encapsulated in polymeric gels, membranes, or micelles in surfactant-stabilized aqueous droplets. The choice of a suitable immobilization method for a given enzyme depends enzyme characteristics, process demands, properties of support, and safety issues, and can be determined by one of skill in the art. Methods for immobilization of enzymes can be found, for example, in Methods of Enzymology, vol. 44, 135, 136, and 137, Academic Press, New York. Accordingly, the invention encompasses an enzymatic composition which comprises one or more solid phase(s), wherein a catalytically active enzyme of the invention is present on the solid phase(s).

The invention further encompasses enzymes of the invention in solid form. Enzymes in solid form or enzyme granulate can be used, for example, in solid detergent and in animal feed. Methods of making solid forms of enzymes are well known in the art, such as but not limited to prilling (spray-cooling in a waxy material), extrusion, agglomeration, or granulation (dilution with an inert material and binders). Solid enzymatic compositions comprising a solid form of an enzyme of the invention, in the form of mixed powder, tablets, and the like, is contemplated.

5.4. Isolation and Use of Antibodies Recognizing Products Encoded by Aspergillus fumigatus Enzyme Genes

Described herein are methods for the production of antibodies capable of specifically recognizing epitopes of one or more of the enzyme gene products described above. Such antibodies can include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

For the production of antibodies to an enzyme gene or gene product, various host animals can be immunized by injection with a enzyme gene protein, or a portion thereof. Such host animals can include but are not limited to rabbits, mice, and rats, to name but a few. Various adjuvants can be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Accordingly, the invention provides a method of eliciting an immune response in an animal, comprising introducing into the animal an immunogenic composition comprising an isolated polypeptide, the amino acid sequence of which comprises at least 6 consecutive residues of one of SEQ ID NO: 3, 6, 9, 12, 15, 18, 21, 24, 27, 30, 33, 36, 39, 42, 45, 48, 51, 54, 57, 60, 63, 66, 69, and 72, as well as the gene product encoded by genomic sequences of SEQ ID NO: 1, 4, 7, 10, 13, 16, 19, 22, 25, 28, 31, 34, 37, 40, 43, 46, 49, 52, 55, 58, 61, 64, 67, and 70, as expressed by Aspergillus fumigatus.

Polyclonal antibodies are heterogeneous populations of antibody molecules derived from the sera of animals immunized with an antigen, such as enzyme gene product, or an antigenic functional derivative thereof. For the production of polyclonal antibodies, host animals such as those described above, can be immunized by injection with enzyme gene product supplemented with adjuvants as also described above. The antibody titer in the immunized animal can be monitored over time by standard techniques, such as with an enzyme linked immunosorbent assay (ELISA) using immobilized polypeptide. If desired, the antibody molecules can be isolated from the animal (e.g., from the blood) and further purified by well-known techniques, such as protein A chromatography to obtain the IgG fraction.

Monoclonal antibodies, which are homogeneous populations of antibodies to a particular antigen, can be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. The hybridoma producing the mAb of this invention can be cultivated in vitro or in vivo. Production of high titers of mAbs in vivo makes this the presently preferred method of production.

Alternative to preparing monoclonal antibody-secreting hybridomas, a monoclonal antibody directed against a polypeptide of the invention can be identified and isolated by screening a recombinant combinatorial immunoglobulin library (e.g. an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

Antibody fragments which recognize specific epitopes can be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed (Huse et al., 1989, Science 246:1275-1281) to allow rapid and easy identification of monoclonal Fab fragments with the desired specificity.

Antibodies of the present invention may also be described or specified in terms of their binding affinity to a enzyme gene product. Preferred binding affinities include those with a dissociation constant or Kd less than 5×10⁻⁶ M, 10⁻⁶M, 5×10⁻⁷ M, 10⁻⁷M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibodies directed against an enzyme gene product or fragment thereof can be used to detect the enzyme gene product in order to evaluate the abundance and pattern of expression of the polypeptide under various environmental conditions, in different morphological forms (mycelium, spores) and stages of an organism's life cycle. Antibodies directed against an enzyme gene product or fragment thereof can be used diagnostically to monitor levels of an enzyme gene product in the tissue of an infected host as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerytrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, antibodies directed against an enzyme gene product or fragment thereof can be used therapeutically to treat an infectious disease by preventing infection, and/or inhibiting growth of the pathogen. Antibodies can also be used to modify the enzyme activity of an enzyme gene product.

5.5. Uses of the Enzymes

5.5.1. Tannases

The present invention encompasses polypeptides having tannase activity. The amino acid sequence of a first polypeptide of the invention having tannase activity is set forth in SEQ ID NO. 3. The polypeptide of SEQ ID NO: 3, herein referred to as tannase 1, is a gene product encoded by the ORF sequence of SEQ ID NO. 2 which is derived from the enzyme gene sequence of SEQ ID NO. 1.

The amino acid sequence of a second polypeptide of the invention having tannase activity is set forth in SEQ ID NO. 6. The polypeptide of SEQ ID NO: 6, herein referred to as tannase 2, is a gene product encoded by the ORF sequence of SEQ ID NO. 4 which is derived from the enzyme gene sequence of SEQ ID NO. 5.

As used herein the terms “tannase 1” and “tannase 2” encompass respectively, not only the polypeptides of SEQ ID NO: 3 and 6, but also all the enzyme gene products related to SEQ ID NO: 1, 2, 4, and 5 as described above in section 5.2, including but not limited to homologs, splice variants, polypeptide fragments, fusion proteins, and functional derivatives, that display tannase activity. In preferred embodiments, homologs of tannase 1 having greater than 48% amino acid sequence identity with tannase 1, and homologs of tannase 2 having greater than 79% amino acid sequence identity with tannase 2, are provided.

Polypeptides having tannase activity have been used in the tea product-making industry. Green tea leaf (as picked) contains colourless polyphenols known as catechins. The four major catechins in green tea leaf are epicatechin and epigallocatechin and the gallated forms of these catechins (bearing a gallic acid (GA) residue), epicatechin-3-gallate and epigallocatechin-3-gallate. The general reaction catalysed by tannase (tannin acylhydrolase, EC 3.1.1.20) is the cleavage of gallate ester linkages, both on gallated catechins and also from other gallated compounds within the leaf. Tannase activity may be measured via a number of assays, the choice of which is not critical to the present invention. For purposes of illustration, tannase activity can be determined by a spectrophotometric assay based on protocatechuic acid p-nitrophenyl ester (Iacazio et al., 2000, J. Microbiol. Methods, 42:209-14), or gallate derivative comprising rhodanine (Sharma et al., 2000, Anal. Biochem. 279:85-89).

Epigallocatechin-3-gallate (EGCG) and epicatechin-3-gallate (ECG) are the most abundant catechins in fresh tea leaves and their gallate ester linkages are cleaved by tannase treatment to yield epicatechin, epigallocatechin and gallic acid. Accordingly, A. fumigatus tannase 1 and/or tarmase 2 can be used to increase the levels of epicatechin, epigallocatechin and gallic acid in a tea extract. Generally, a method for modulating the amount of compounds that comprise a gallate ester linkage in a composition comprising contacting the composition with an enzymatic composition which comprises tannase 1 and/or tannase 2, is provided.

During oxidative fermentation of green leaf to produce black tea (either solid state fermentation to produce black leaf or slurry fermentation to produce black tea extracts) the catechins undergo oxidative biotransformations, through their quinones, into dimeric compounds known as theaflavins and higher molecular weight compounds known as thearubigins. Theaflavins and thearubigins are responsible for the orange and brown colours of black tea infusions and products as well as making significant contributions to the astringency and body of the made tea. The oxidative polymerisations are a combination of biochemical oxidations mediated by polyphenol oxidase and/or peroxidase enzymes. Theaflavin and theaflavins have been recognized to affect tea flavor and color. Most theaflavins have antioxidant properties and are therefore of great interest to the food and health industries. Tannase treatment of tea leaf extracts at various stages of tea product manufacturing results in a change in the levels of theaflavins and thearubigins. Accordingly, A. fumigatus tannase 1 and/or tannase 2 can be used to modulate the levels of theaflavins and thearubigins in a tea extract. For example, the tannase 1 and/or tannase 2 of the invention can be used in the processes of tea product manufacturing as described in U.S. Pat. No. 6,113,965.

Black tea extracts are normally produced by a hot or boiling water extraction process. However, the black tea extracts, and particularly dried black tea extract, when made to beverage concentrates, usually become turbid if the beverage or the extract is allowed to cool to room temperature or lower. This turbidity is caused by material present in the original black tea (tea solids which are extracted by hot water, but which are insoluble in cold water). This precipitate, known as “tea cream”, is separated from the infusion, for example by centrifugation. This clouding or creaming, however, has been a serious problem in the preparation of a stable commercial tea concentrate and in the acceptance by the consumer of soluble instant tea powders, particularly of instant ice tea products. Tannase has been used to remove this tea cream or to solubilize the cold water-insoluble constituents of a hot water extract of tea. Accordingly, A. fumigatus tannase 1 and/or tannase 2 can be used to solubilize the cold water-insoluble constituents of a hot water extract of tea, and generally, to improve the clarity of tea products. For example, the tannase 1 and/or tannase 2 of the invention can be used in the processes such as those described in British Patents GB-B-1,413,351 and GB-B-1,380,135, U.S. Pat. Nos. 4,639,375; 5,258,188; 5,445,836; 5,925,389.

In various embodiments, the tannase 1 and/or tannase 2 of the invention can be used to increase the yield of tea liquor from tea leaves, to improve the color, flavor, and health benefits of a tea product, particularly an instant tea product. The enzymes can also be used in wine making. The invention further encompasses an enzyme composition comprising tannase 1, tannase 2, or both, in free form or in an immobilized form. The enzyme composition may contain additional enzymes, such as but not limited to polyphenol oxidases, cellulases, hernicellulases, pectinases, or laccases.

5.5.2. Cellulase

The present invention encompasses polypeptides having cellulase activity. The amino acid sequence of a polypeptide of the invention having cellulase activity is set forth in SEQ ID NO. 9. The polypeptide of SEQ ID NO: 9, herein referred to as cellulase 1, is a gene product encoded by the ORF sequence of SEQ ID NO. 8 which is derived from the enzyme gene sequence of SEQ ID NO. 7. As used herein the terms “cellulase 1” encompasses respectively, not only the polypeptide of SEQ ID NO: 9, but also all the enzyme gene products related to SEQ ID NO: 7 and 8 as described above in section 5.2, including but not limited to homologs, splice variants, polypeptide fragments, fusion proteins, and functional derivatives, that display cellulase activity. In a preferred embodiment, homologs of cellulase 1 having greater than 76% amino acid sequence identity with cellulase 1 are provided.

The general reaction catalysed by cellulase is that of an endoglucanases (E.C. 3.2.1.4), cellobiohydrolases (also called exoglucanase, E.C. 3.2.1.91), or a β-glucosidases.(also called cellobiase, E.C. 3.2.1.21). Endoglucanases hydrolyze β-glycoside bonds internally and randornly along the cellulose chains whereas cellobiohydrolases remove cellobiose molecules from the reducing and non-reducing ends of the chains. β-Glucosidases hydrolyze the cellobiose to two molecules of glucose, and therefore eliminate the inhibition of cellobiose on cellobiohydrolases and endoglucanases. The presence of all three components in a composition is generally known as a complete cellulase system which can efficiently convert crystalline cellulose to glucose. Accordingly, A. fumigatus cellulase 1 can be used in methods that require hydrolysis of cellulose. Generally, a method for modulating the amount of cellulose in a composition comprising contacting the composition with an enzymatic composition which comprises cellulase 1, is provided. Cellulase activity may be measured via a number of assays, the choice of which is not critical to the present invention. For purposes of illustration, cellulase activity may be determined by a colorimetric assay based on a ferricyanide-molybdoarsenic acid reagent (Holm, 1978, Anal. Biochem. 84:522-532).

Polypeptides having cellulase activity have been included in detergent compositions for the purposes of enhancing the cleaning ability of the composition. Accordingly, A. fumigatus cellulase 1 can be used as a component of a detergent composition, and in methods of laundering garments in conjunction with other enzymes and surfactants. For example, the cellulase 1 of the invention can be used in the methods or be incorporated into the compositions such as those described in U.S. Pat. Nos. 5,904,736; 5,883,066; 6,020,293; 6,235,697; Great Britain Application Nos. 2,075,028, 2,095,275 and 2,094,826. Cellulase can be used to remove a greyish cast on washed garments containing on the surface disrupted and disordered fibrils caused by mechanical action. Cellulases have also been used for denim garment finishing, to achieve softness and the fashionable wom look traditionally obtained by stone-washing and acid washing. Accordingly, A. fumigatus cellulase 1 can be used for altering the properties of textile fibers including but not limited to cotton. The properties affected by cellulase treatment include but are limited to wettability, absorbancy, softness to the touch, optical properties relating to the reflection of light by dyes in colored fibers on the surface of garments. For example, the cellulase 1 of the invention can be used and incorporated into the compositions as described in U.S. Pat. Nos. 4,738,682; 5,874,293; 5,908,472; 5,916,798; 5,919,697; 6,066,494,. In various embodiments, the cellulase 1 of the invention can be used as a component of a detergent, as a cleaning agent, as a softening agent, or as a color restoring agent.

Cellulase has also been used to preserve and enhance the nutritive value of forage for silage and to improve the palatability, digestibility and rate of digestion of treated forage by ruminants. Accordingly, A. fumigatus cellulase 1 can be used in methods for reducing the amounts of cellulose in food products or animal feed. The cellulase can be used as additives for feed, digestants, and waste management agents. For example, the cellulase 1 of the invention can be used and included in compositions as described in U.S. Pat. Nos. 5,948,454; 6,042,853.

The invention further encompasses an enzyme composition comprising cellulase 1 in free form or in an immobilized form. The enzyme composition may contain additional enzymes, such as but not limited to other types of cellulases, hemicellulases, tannases, lipases, or pectinases. In a preferred embodiment, the enzyme composition comprising cellulase 1 is a complete cellulase system.

5.5.3. Glucose Oxidases

The present invention encompasses polypeptides having glucose oxidase activity. The amino acid sequence of a first polypeptide of the invention having glucose oxidase activity is set forth in SEQ ID NO: 12. The polypeptide of SEQ ID NO: 12, herein referred to as glucose oxidase 1, is a gene product encoded by the ORF sequence of SEQ ID NO: 11 which is derived from the enzyme gene sequence of SEQ ID NO. 10. The amino acid sequence of a second polypeptide of the invention having glucose oxidase activity is set forth in SEQ ID NO. 15. The polypeptide of SEQ ID NO: 15, herein referred to as glucose oxidase 2, is a gene product encoded by the ORF sequence of SEQ ID NO. 14 which is derived from the enzyme gene sequence of SEQ ID NO. 13. The amino acid sequence of a third polypeptide of the invention having glucose oxidase activity is set forth in SEQ ID NO. 18. The polypeptide of SEQ ID NO: 6, herein referred to as glucose oxidase 3, is a gene product encoded by the ORF sequence of SEQ ID NO. 17 which is derived from the enzyme gene sequence of SEQ ID NO. 16. As used herein the terms “glucose oxidase 1”, “glucose oxidase 2” and “glucose oxidase 3” encompasses respectively, not only the polypeptides of SEQ ID NO: 12, 15, and 18, but also all the enzyme gene products related to SEQ ID NO: 10, 11, 13, 14, 16, and 17 as described above in section 5.2, including but not limited to homologs, splice variants, polypeptide fragments, fusion proteins, and functional derivatives, that display glucose oxidase activity. In preferred embodiments, homologs of glucose oxidase 1 having greater than 34% amino acid sequence identity with glucose oxidase 1, homologs of glucose oxidase 2 having greater than 29% amino acid sequence identity with glucose oxidase 2, and homologs of glucose oxidase 3 having greater than 34% amino acid sequence identity with glucose oxidase 3, are provided.

Enzymes having glucose oxidase activity catalyze the oxidation of glucose to gluconic acid with the concomitant production of hydrogen peroxide. Accordingly, A. fumigatus glucose oxidase 1, glucose oxidase 2, and/or glucose oxidase 3 can be used in methods for producing gluconic acid and hydrogen peroxide. Moreover, the A. fumigatus glucose oxidases can individually or in combination be used to modulate the levels of oxygen, especially in a defined volume of space or in a modified atmosphere, such as but not limited to the spaces between food products, beverages and the packaging. The enzyme(s) can be used as a component of an antioxidant system, or in methods for removing oxygen so as to minimize detrimental oxidative processes in food. For example, the glucose oxidase 1, glucose oxidase 2, and/or glucose oxidase 3 of the invention can be used in the kind of processes described in U.S. Pat. No. 4,996,062 and 6,093,436.

Generally, a method for modulating the amount of glucose or oxygen in a composition comprising contacting the composition with an enzymatic composition which comprises glucose oxidase 1, glucose oxidase 2 and/or glucose oxidase 3, is provided. Glucose oxidase activity may be measured via a number of assays, the choice of which is not critical to the present invention. For purposes of illustration, glucose oxidase activity may be determined by measuring a decrease in glucose using a colorimetric assay as described in Blake and McLean (1989, Anal. Biochem. 177:156-160).

Glucose monitoring is commonly practiced by diabetic individuals to measure the level of glucose in a small amount of blood using a device. Many of these devices detect glucose in a blood sample electrochemically, by detecting the oxidation of blood glucose using glucose oxidase, provided as part of a disposable, single-use electrode system. Glucose monitoring is also performed routinely in various industrial processes such as starch conversion, and fermentation, where glucose is either used as a starting material or generated as an intermediate, a by-product, or an end-product. Accordingly, A. fumigatus glucose oxidase 1, glucose oxidase 2, and/or glucose oxidase 3 can be used in methods for detecting the presence of or measuring the concentration of glucose in a sample, such as body fluids, and fluid streams in industrial processes. For example, the glucose oxidase 1, glucose oxidase 2, and/or glucose oxidase 3 of the invention can be used in the devices and methods disclosed in European Patent No. 0 127 958, and U.S. Pat. Nos. 5,141,868; 5,286,362; 5,288,636; 5,437,999; and 6,241,862.

A. fumigatus glucose oxidases can also be as a bleach for dyes that have leached out of fabrics to prevent dye transfer in a laundering process, such as the methods described in WO 91/05839. In various embodiments, the glucose oxidase 1, glucose oxidase 2 and/or glucose oxidase 3 of the invention can be used in detergents, in desugaring eggs, in the removal of oxygen from beverages, moist food products, flavors, and hermetically sealed food packages, and in the detection and estimation of glucose in industrial solutions, and in body fluids such as blood and urine. The invention further encompasses an enzyme composition comprising the glucose oxidase 1, glucose oxidase 2 and/or glucose oxidase 3, in free form or in an immobilized form. The invention further encompasses a mechanical composition comprising the glucose oxidase 1, glucose oxidase 2 and/or glucose oxidase 3, which can be a device, or a form suitable for use in a device (e.g., test strips).

5.5.4. Phytase

The present invention encompasses polypeptides having phytase activity. The amino acid sequence of a polypeptide of the invention having phytase activity is set forth in SEQ ID NO. 24. The polypeptide of SEQ ID NO: 24, herein referred to as phytase 1, is a gene product encoded by the ORF sequence of SEQ ID NO. 23 which is derived from the enzyme gene sequence of SEQ ID NO. 22. As used herein the terms “phytase 1” encompasses respectively, not only the polypeptide of SEQ ID NO: 24, but also all the enzyme gene products related to SEQ ID NO: 23 and 22 as described above in section 5.2, including but not limited to homologs, splice variants, polypeptide fragments, fusion proteins, and functional derivatives, that display phytase activity. In a preferred embodiment, homologs of phytase 1 not from A. fumigatus having greater than 27% amino acid sequence identity with phytase 1 is provided.

A phytase is an enzyme which catalyzes the hydrolysis of phytate or myoinositol 1,2,3,4,5,6-hexakis dihydrogen phosphate (or for short myo-inositol hexakisphosphate) to (1) myo-inositol and/or (2) mono-, di-, tri-, tetra- and/or penta-phosphates thereof and (3) inorganic phosphate. There are primarily two types of phytases: 3-phytase (myo-inositol hexaphosphate 3-phosphohydrolase, EC 3.1.3.8) and 6-phytase (myo-inositol hexaphosphate 6-phosphohydrolase, EC 3.1.3.26). The 3-phytase hydrolyses first the ester bond at the 3-position, whereas the 6-phytase hydrolyzes first the ester bond at the 6-position. Accordingly, A. fumigatus phytase 1 can be used in degrading phytates, in methods for producing myo-inositol and/or its mono-, di-, tri-, tetra- and/or penta-phosphates from phytates, in methods of modulating the amount of myo-inositol phosphates, or in methods for removing inorganic phosphorous from various myo-inositol phosphates. Phytase activity may be measured via a number of assays, the choice of which is not critical to the present invention. For purposes of illustration, phytase activity may be determined by measuring the amount of enzyme which liberates inorganic phosphorous from 1.5 mM sodium phytate at the rate of 1 μmol/min at 37 C. and at pH 5.50.

A. fumigatus phytase 1 of the present invention may be applied to a variety of processes which require the conversion of phytate to inositol and inorganic phosphate. Phytic acid is the primary source of inositol and the primary storage form of phosphate in plant seeds. Seeds, cereal grains and legumes are important components of food and feed preparations, in particular of animal feed preparations. But also in human food cereals and legumes are becoming increasingly important. The phosphate moieties of phytic acid chelates divalent and trivalent cations such as metal ions, including the nutritionally essential ions of calcium, iron, zinc and magnesium as well as the trace minerals manganese, copper and molybdenum. However, phytic acid and its salts, phytates, are often not metabolized, since they are not absorbable from the gastrointestinal system. As a result, food and feed preparations need to be supplemented with inorganic phosphate and other nutritionally essential ions such as iron and calcium. Still further, since phytic acid is not metabolized, the phytate phosphorus is excreted with the manure, resulting in an undesirable phosphate pollution of the environment. Accordingly, A. fumigatus phytase 1 can be used in methods for increasing the nutritive value of food or feed substances. For example, the phytase 1 of the invention can be used and incorporated into the compositions such as those described in U.S. Pat. Nos. 3,297,548; 5,436,156; 6,063,431; 6,221,644.

In various embodiments, the phytase 1 of the invention can be used as a component of animal feed additives, especially animal feed additives for monogastric animals, such as pigs and poultry. Phytase activity in feed can be determined by a colorimetric assay as described in Engelen et al. (2001, J. AOAC Int. 84:629-633). A. fumigatus phytase 1 can also be used in other industrial processes using substrates that contain phytate such as the starch industry and in fermentation industries, such as the brewing industry. The invention further encompasses an enzyme composition comprising phytase 1 in free form or in an immobilized form. The enzyme composition may contain additional enzymes, such as but not limited to other phytases, and cellulases. The invention also encompasses animal feed compositions comprising plant seeds and A. fumigatus phytase 1.

5.5.5. β-Galactosidases

The present invention encompasses polypeptides having β-galactosidase activity.

The amino acid sequence of a first polypeptide of the invention having β-galactosidase activity is set forth in SEQ ID NO. 27. The polypeptide of SEQ ID NO: 27, herein referred to as β-galactosidase 1, is a gene product encoded by the ORF sequence of SEQ ID NO. 26 which is derived from the enzyme gene sequence of SEQ ID NO. 25. The amino acid sequence of a second polypeptide of the invention having β-galactosidase activity is set forth in SEQ ID NO. 30. The polypeptide of SEQ ID NO: 27, herein referred to as β-galactosidase 2, is a gene product encoded by the ORF sequence of SEQ ID NO. 29 which is derived from the enzyme gene sequence of SEQ ID NO. 28. As used herein the terms “β-galactosidase 1 ” and “β-galactosidase 2” encompasses respectively, not only the polypeptides of SEQ ID NO: 27 and 30, but also all the enzyme gene products related to SEQ ID NO: 25, 26, 28, and 29 as described above in section 5.2, including but not limited to splice variants, polypeptide fragments, fusion proteins, and derivatives, that display β-galactosidase activity. In preferred embodiments, homologs of β-galactosidase 1 having greater than 54% amino acid sequence identity with β-galactosidase 1, and homologs of β-galactosidase 2 having greater than 70% amino acid sequence identity with galactosidase 2, are provided.

β-galactosidase (also known as lactase) is an enzyme capable of hydrolyzing lactose into galactose and glucose, both of which are sweeter and more digestible by humans. Accordingly, A. fumigats β-galactosidase 1 and/or β-galactosidase 2 can be used in methods for producing galactose and/or glucose from lactose, and methods for modulating the level of lactose, galactose and glucose in a composition. For example, cheese whey contains large amounts of lactose, and can thus be used as a source of galactose or glucose after treatment with the β-galactosidase 1 and/or β-galactosidase 2 of the invention.

Food-grade lactase enzyme preparations have been commercially available. To reduce symptoms of lactose maldigestion, such preparations have been used to hydrolyze lactose in milk prior to consumption or taken in the form of a pill. See, e.g., Corazza et al., Aliment. Pharmacol. Therap. 6:61-66 (1992); Solomons et al., Am. J. Clin. Nutr. 41:222-227 (1985); Rosado et al., J. Am. College Nutr. 5:281-290 (1986); Paige et al., Am. J. Clin. Nutr. 28:818-822 (1975). Accordingly, the β-galactosidase 1 and/or β-galactosidase 2 can be used to make food products that are lactose-reduced or lactose-free, e.g., lactose-free milk. The invention further encompasses an enzyme composition comprising β-galactosidase 1, β-galactosidase 2, or both, in free form or in an immobilized form.

5.5.6. Invertase

The present invention encompasses polypeptides having invertase (or sucrase) activity. The amino acid sequence of a polypeptide of the invention having invertase activity is set forth in SEQ ID NO. 36. The polypeptide of SEQ ID NO: 36, herein referred to as invertase 1, is a gene product encoded by the ORF sequence of SEQ ID NO. 35 which is derived from the enzyme gene sequence of SEQ ID NO. 34. As used herein the terms “invertase 1 ” encompasses respectively, not only the polypeptide of SEQ ID NO: 36, but also all the enzyme gene products related to SEQ ID NO: 35 and 34 as described above in section 5.2, including but not limited to splice variants, polypeptide fragments, fusion proteins, and derivatives, that display invertase activity. In a preferred embodiment, homologs of invertase 1 having greater than 29% amino acid sequence identity with invertase 1 is provided.

The reaction catalysed by invertase is the conversion of sucrose to the hexose sugars glucose and fructose. Accordingly, A. fumigates invertase 1 can be used in methods for making glucose, methods for making fructose, or methods for modulating the levels of sucrose, glucose and fructose in a composition. For example, cane molasses is a by-product containing sucrose which is produced in the sugar-manufacturing industry. Invertase 1 of the invention can be used in a process that convert the sucrose in the molasses into hexoses so that the molasses can be used as a fermentation starting material for the manufacturing of other valuable chemicals, such as amino acids. See, for example, U.S. Pat. Nos. 4,774,183; and 4,543,330.

The invention further encompass an enzyme composition comprising invertase 1 in free form or in an immobilized form. Invertase activity may be measured via a number of assays, the choice of which is not critical to the present invention. For purposes of illustration, dehydrogenase-linked assays or a colorimetric assay as described in Carins (1987, Anal. Biochem. 167:270-278) can be used.

5.5.7. Lipase

The present invention encompasses polypeptides having lipase activity. The amino acid sequence of a polypeptide of the invention having lipase activity is set forth in SEQ ID NO. 39. The polypeptide of SEQ ID NO: 39, herein referred to as lipase 1, is a gene product encoded by the ORF sequence of SEQ ID NO.38 which is derived from the enzyme gene sequence of SEQ ID NO.37. As used herein the terms “lipase 1” encompasses respectively, not only the polypeptide of SEQ ID NO: 39, but also all the enzyme gene products related to SEQ ID NO: 38 and 37 as described above in section 5.2, including but not limited to homologs, splice variants, polypeptide fragments, fusion proteins, and functional derivatives, that display lipase activity. In a preferred embodiment, homologs of lipase 1 having greater than 61% amino acid sequence identity with lipase 1 is provided. Lipases are a group of enzymes belonging to the esterases, and are also called glyceroester hydrolases or acylglycerol-acylhydrolases. Lipases are employed for their ability to modify the structure and composition of triglyceride oils and fats by hydrolysis, esterification and transesterification reactions. These are equilibrium reactions which in one direction result into hydrolysis of triglycerides into free fatty acids and glycerol, mono- or diglycerides, and in the other direction result into re-esterification of glycerol, monoglycerides and diglycerides into triglycerides. Accordingly, A. fumigatus lipase 1 can be used in methods for degrading oils or fats, or producing fatty acids and alcohols from fats or oils, or in methods for modulating the amounts of triglycerides, in a composition. Many known lipases are characterized by a broad substrate spectrum of activity combined with frequently very high stereoselectivity. The end-products of such a lipase reaction, such as monoesters, maybe used as chiral precursors for a variety of compounds, such as non-naturally occurring amino acids and chiral polyesters. Accordingly, A. fumigatus lipase 1 can be used in methods for preparing fatty acids, esters, or alcohols of high optical purity. See, for example, U.S. Pat. Nos. 6,201,147; 6,210,956. Lipase activity may be measured via a number of assays, the choice of which is not critical to the present invention. For example, lipase activity may be determined by the assay of McKellar (1986, J. Dairy Res. 53:117-127).

Moreover, lipases have been included in detergent compositions for improved cleaning performance, e.g. used in the enhancement of removal of triglycerides containing soils and stains from fabrics. Lipases have also been used in desizing of the thread of fabric when the size used comprises oils or fat. Accordingly, A. fumigatus lipase 1 can be used in degrading fat and oils in the laundry or textile industry, or added to detergent compositions. For example, the lipase 1 of the invention can be used and incorporated into the compositions as described in U.S. Pat. Nos. 4,769,173; 5,069,809; 6,071,356; and PCT application WO94/03578.

The invention further encompasses an enzyme composition comprising lipase 1 in free form or in an immobilized form. The enzyme composition may contain additional enzymes, such as but not limited to other types of hemicellulases, tannases, xylanases, lipases, or pectinases.

5.5.8. Amylases and Glucoamylases

The present invention encompasses polypeptides having amylase activity and glucoamylase activity. The amino acid sequence of a first polypeptide of the invention having α-amylase activity is set forth in SEQ ID NO. 42. The polypeptide of SEQ ID NO: 42, herein referred to as α-amylase 1, is a gene product encoded by the ORF sequence of SEQ ID NO: 41 which is derived from the enzyme gene sequence of SEQ ID NO. 40. The amino acid sequence of a second polypeptide of the invention having amylase activity is set forth in SEQ ID NO. 45. The polypeptide of SEQ ID NO: 45, herein referred to as α-amylase 2, is a gene product encoded by the ORF sequence of SEQ ID NO. 44 which is derived from the enzyme gene sequence of SEQ ID NO. 43. The amino acid sequence of a third polypeptide of the invention having amylase activity is set forth in SEQ ID NO. 48. The polypeptide of SEQ ID NO: 48, herein referred to as α-amylase 3, is a gene product encoded by the ORF sequence of SEQ ID NO. 47 which is derived from the enzyme gene sequence of SEQ ID NO. 46. As used herein the terms “α-amylase 1”, “α-amylase 2” and “α-amylase 3” encompasses respectively, not only the polypeptides of SEQ ID NO: 42, 45, and 48, but also all the enzyme gene products related to SEQ ID NO: 40, 41, 43, 44, 46, and 47 as described above in section 5.2, including but not limited to splice variants, polypeptide fragments, fusion proteins, and derivatives, that display amylase activity. In preferred embodiments, homologs of α-amylase 1 having greater than 78% amino acid sequence identity with α-amylase 1, homologs of α-amylase 2 having greater than 70% amino acid sequence identity with α-amylase 2, and homologs of α-amylase 3 having greater than 50% amino acid sequence identity with α-amylase 3, are provided.

The amino acid sequence of a first polypeptide of the invention having glucoamylase activity is set forth in SEQ ID NO. 21. The polypeptide of SEQ ID NO: 21, herein referred to as glucoamylase 1, is a gene product encoded by the ORF sequence of SEQ ID NO: 20 which is derived from the enzyme gene sequence of SEQ ID NO. 19. The amino acid sequence of a second polypeptide of the invention having amylase activity is set forth in SEQ ID NO. 33. The polypeptide of SEQ ID NO: 33, herein referred to as glucoamylase 2, is a gene product encoded by the ORF sequence of SEQ ID NO. 32 which is derived from the enzyme gene sequence of SEQ ID NO. 31. The amino acid sequence of a third polypeptide of the invention having amylase activity is set forth in SEQ ID NO. 54. The polypeptide of SEQ ID NO: 54, herein referred to as glucoamylase 3, is a gene product encoded by the ORF sequence of SEQ ID NO. 53 which is derived from the enzyme gene sequence of SEQ ID NO. 52. As used herein the terms “glucoamylase 1”, “glucoamylase 2” and “glucoamylase 3” encompasses respectively, not only the polypeptides of SEQ ID NO: 21, 33, and 54, but also all the enzyme gene products related to SEQ ID NO: 19, 20, 31, 32, 52, and 53 as described above in section 5.2, including but not limited to splice variants, polypeptide fragments, fusion proteins, and derivatives, that display amylase activity. In preferred embodiments, homologs of glucoamylase 1 having greater than 58% amino acid sequence identity with glucoarnylase 1, homologs of glucoamylase 2 having greater than 51% amino acid sequence identity with glucoamylase 2, and homologs of glucoamylase 3 having greater than 68% amino acid sequence identity with glucoamylase 3, are provided.

Amylases cleave the α-1,4-glycosidic linkages of starch. Glucoamylases hydrolyse the terminal glucose monomers. Amylases and glucoamylase (also known as amyloglucosidase) are used as processing aid to convert starch-bearing raw materials (e.g., corn, potato, wheat, cassaya, barley) to products useful to the food industry, such as starches, starch derivatives and starch saccharification products of different sweetness. The primary steps of starch conversion are liquefaction, saccharification, and isomerization. The first step after a starch slurry is prepared, is heating and enzyme treatment. Thermostable amylases have been used to cleave the α-1,4-glycosidic linkages of pregelatinized starch to reduce the visocosity of the slurry, and to produce maltodextrins of low dextrose-equivalent values (DE <25). Maltodextrins are used as blandtasting functional ingredients, e.g., fillers, stabilizers, thickeners, paste, glues. Accordingly, the A. fumigatus α-amylase 1, α-amylase 2 and/or α-amylase 3, which are thermostable can be used in methods for gelatinizing starch, starch liquefaction, methods for reducing viscosity of a starch slurry, and methods for producing maltodextrins (DE <25, or DE of 8-12, 10-20 or 15-25). Generally, a method for modulating the amounts of starches or maltodextins in a composition comprising contacting the composition with an enzymatic composition which comprises α-amylase 1, α-amylase 2 and/or α-amylase 3, is provided. The invention encompasses an enzyme composition comprising the α-amylase 1, α-amylase 2 and/or α-amylase 3, in free form or in an immobilized form. In preferred embodiments, the α-amylase 1, α-amylase 2 and/or α-amylase 3 of the invention are present in an enzyme composition further comprising other α-amylases, such as bacterial α-amylases, preferably thermostable α-amylases including those derived from Bacillus subtilis and B. licheniformis. α-amylase activity may be measured via a number of assays, the choice of which is not critical to the present invention. For purposes of illustration, α-amylase activity may be determined the colorimetric assay of Winn-Deen et al. (1988, Clin. Chem. 34:2005-8), or the colorimetric and electron spin resonance spectroscopy (ESR) methods described in Marcazzan (1999, J. Biochem. Biophys. Methods, 38:191-202).

The next step of starch conversion is saccharification which can result in the near-total conversion of starch to glucose. Fungal glucoamylases obtained from A. niger, A. oryzae, A. awamori, which display an exoamylase activity and a low α-1,6-glycosidic cleavage activity (i.e., debranching activity) have been used to make glucose syrup or maltoe syrup with a high DE value (DE>40). Accordingly, the A. fumigatus glucoamylase 1, glucoamylase 2 and/or glucoamylase 3, can be used in methods for saccharification of starch, methods for saccharification of maltodextrin, methods for producing high dextrose syrup, such as high DE maltose syrup (DE>40, and up to 50-55 or 55-70), and methods for producing glucose syrup. Generally, a method for modulating the amounts of starches or maltodextrins in a composition comprising contacting the composition with an enzymatic composition which comprises glucoamylase 1, glucoamylase 2 and/or glucoamylase 3, is provided. The invention encompasses an enzyme composition comprising the glucoamylase 1, glucoamylase 2 and/or glucoamylase 3, in free form or in an immobilized form. In preferred embodiments, the glucoamylase 1, glucoamylase 2 and/or glucoamylase 3 of the invention are present in an enzyme composition further comprising other glucoamylases, β-amylases, and pullulanases.

For example, the α-amylases and glucoamylases can be used in starch converison processes such as those described in U.S. Pat. Nos. 4,132,595; 4,933,279; 5,180,699; 5,322,778; 5,445,990; and 5,935,826.

Fungal α-amylases have also been used along with proteases by the baking industry to affect the functional properties of dough and enhances characteristics that are desirable for the automated production of baked goods. Added α-amylases can increase the levels of fermentable monosaccharides and disaccharides in the dough which enhance the growth of baker's yeast. Accordingly, the A. fumigatus α-amylase 1, α-amylase 2 and/or α-amylase 3, can be used in methods for supplementing the amylolytic activity in flour or dough, methods for reducing the viscosity of dough, methods for increasing bread volume, and methods for improving storage properties of baked goods. The invention further encompasses an enzyme composition comprising the α-amylase 1, α-amylase 2 and/or α-amylase 3 of the invention and proteases. For example, the α-amylases can be used in processes for making baked products as described in U.S. Pat. Nos. 4,654,216; 5,352,473; 5,338,552 and 6,068,864.

Fungal α-amylases and glucoamylases have also been used in the brewing industry during the various stages of the brewing process, or in specific processes, such as barley brewing. The enzymes can be added during the mashing step to generate fermentable sugars from starch in the wort. The enzymes, α-amylases in particular, are used to produce low-carbohydrate “light” beer while glucoamylases maybe added to produce a sweet beer. Fungal α-amylases may be added to promote hydrolysis of residual starch which may contribute to turbidity in the final product. The enzymes can also be added to produce a highly carbonated brewed beverage by hydrolysing the residual starch for a second fermentation. The A. fumigatus α-amylase 1, α-amylase 2, α-amylase 3, glucoarnylase 1, glucoarnylase 2 and/or glucoamylase 3 can be used in any of these processes along with or in place of the fungal enzymes currently used. For example, the α-amylases and glucoamylases can be used in fermentation processes as described in U.S. Pat. Nos. 3,988,204; 5,021,246; and 5,048,385.

α-amylases have also been used in laundry detergents. The enzyne(s), preferably thernostable, catalyse the degradation of starch stains, and improve cleaning by hydrolysing the starch that binds other dirt and stains to fabric. Accordingly, the A. fumigatus α-amylase 1, α-amylase 2 and/or α-amylase 3, can be used as an additive in detergent compositions, and in methods for laundering fabric or dishwashing. The invention further encompasses a detergent composition comprising the α-amylase 1, α-amylase 2 and/or α-amylase 3 of the invention, surfactants, and other enzymes such as but not limited to proteases, lipases, and cellulases. For example, the α-amylases can be used in cleaning processes as described in U.S. Pat. Nos. 5,851,973; 5,972,040; 6,140,293; and 6,147,045.

5.5.9. Laccase

The present invention encompasses polypeptides having laccase activity. The amino acid sequence of a polypeptide of the invention having laccase activity is set forth in SEQ ID NO. 51. The polypeptide of SEQ ID NO: 51, herein referred to as laccase 1, is a gene product encoded by the ORF sequence of SEQ ID NO. 50 which is derived from the enzyme gene sequence of SEQ ID NO. 49. As used herein the terms “laccase 1” encompasses respectively, not only the polypeptide of SEQ ID NO: 51, but also all the enzyme gene products related to SEQ ID NO: 50 and 49 as described above in section 5.2, including but not limited to splice variants, polypeptide fragments, fusion proteins, and derivatives, that display laccase activity. In a preferred embodiment, homologs of laccase 1 having greater than 46% amino acid sequence identity with laccase 1 is provided.

Known laccases (benzenediol:oxygen oxidoreductases; E.C. 1.10.3.2) are multi-copper containing enzymes that catalyze the oxidation of phenolics. Laccase-mediated oxidations produce aryloxy-radical intermediates from a phenolic substrate which result in the formation of dimeric to polymeric reaction products. Known laccases exhibits a wide range of substrate specificity. A major problem with the use of known laccases are their poor storage stability at temperatures above room temperature, especially at 40° C. The laccase of the invention is thermostable and can thus be used in many applications that require temperature above room temperature. Laccase activity can be determined by any methods known in the art, such as syringaldazine oxidation monitored at 530 nm, 10-(2-hydroxyethyl)-phenoxazine (HEPO) oxidation which can be monitored photometrically at 528 nm. (G. Cauquil in Bulletin de la Society Chemique de France, 1960, p. 1049), or oxidation of 2,2′-azinobis-(3-ethybenzthiazoline-6-sulfonic acid) (ABTS). Generally, a method for modulating the amounts of oxidated phenolic compounds in a composition comprising contacting the composition with an enzymatic composition which comprises laccase 1, is provided.

The A. fumigatus laccase 1 may be used in a number of different industrial processes. These processes include polymerization of lignin, both Kraft and lignosulfates, in solution, in order to produce a lignin with a higher molecular weight. For example, laccase 1 of the invention can be used in processes such as those disclosed in U.S. Pat. No. 4,432,921; EP 0 275 544; and PCT/DK93/00217, 1993. Laccase 1 can also be useful in the copolymerization of lignin with low molecular weight compounds, such as is described by Milstein et al., 1994, Appl. Microbiol. Biotechnol. 40: 760-767.

The laccase 1 of the present invention can also be used for depolymerization of lignin in Kraft pulp, thereby producing a pulp with lower lignin content. This use of laccase is an improvement over the current use of chlorine for depolymerization of lignin, which leads to the production of chlorinated aromatic compounds, which are an environmentally undesirable by-product of paper mills. Such uses are described in, for example, U.S. Pat. No. 6,023,065; Current Opinion in Biotechnology 3: 261-266, 1992; Journal of Biotechnology 25: 333-339,1992; Hiroi et al., 1976, Svensk Papperstidning 5:162-166, 1976.

Laccase 1 of the invention can also be used in the oxidation of dyes or dye precursors and other chromophoric compounds that leads to decolorization of the compounds. This can be particularly advantageous in a situation in which a dye transfer between fabrics is undesirable, e.g., in the textile industry and in the detergent industry. Methods for bleaching, dye transfer inhibition and dye oxidation using a laccase can be found in U.S. Pat. No. 5,752,890; WO 96/12845; WO 96/12846; WO 92/01406; WO 92/18683; WO 92/18687; WO 91/05839; EP 0495836; Tsujino et al., 1991, J. Soc. Chem. 42: 273-282; which are incorporated herein by reference. Polypeptides having laccase activity can also be used in detergent compositions for the purposes of enhancing the cleaning ability of the composition. Accordingly, A. fumigatus laccase 1 can be used as a component of a detergent composition, and in methods of laundering garments in conjunction with other enzymes and surfectants. For example, the laccase 1 of the invention can be used as described in WO 95/01426.

The present laccase 1 can also be used for the polymerization or oxidation of phenolic compounds present in liquids. An example of such utility is the treatment of juices, such as apple juice, so that the laccase will accelerate a precipitation of the phenolic compounds present in the juice, thereby producing a more stable juice. Laccase 1 of the present invention can also useful in soil detoxification (Nannipieri et al., 1991, J. Environ. Qual. 20: 510-517; Dec and Bollag, 1990, Arch. Environ. Contam. Toxicol. 19: 543-550). In various embodiments, the laccase 1 of the invention can be used in lignin modification, paper strengthening, dye transfer inhibition in detergents, phenol polymerization, juice manufacture, phenol resin production, and waste water treatment. The invention further encompasses an enzyme composition comprising laccase 1, or both, in free form or in an immobilized form. The enzyme composition may contain additional enzymes, such as but not limited to polyphenol oxidases, cellulases, hemicellulases, and pectinases.

5.5.10. Polygalacturonases

The present invention encompasses polypeptides having polygalacturonase activity. The amino acid sequence of a first polypeptide of the invention having polygalacturonase activity is set forth in SEQ ID NO. 57. The polypeptide of SEQ ID NO: 57, herein referred to as polygalacturonase 1, is a gene product encoded by the ORF sequence of SEQ ID NO: 56 which is derived from the enzyme gene sequence of SEQ ID NO. 55. The amino acid sequence of a second polypeptide of the invention having polygalacturonase activity is set forth in SEQ ID NO. 60. The polypeptide of SEQ ID NO: 60, herein referred to as polygalacturonase 2, is a gene product encoded by the ORF sequence of SEQ ID NO. 59 which is derived from the enzyme gene sequence of SEQ ID NO. 58. The amino acid sequence of a third polypeptide of the invention having polygalacturonase activity is set forth in SEQ ID NO. 63. The polypeptide of SEQ ID NO: 63, herein referred to as polygalacturonase 3, is a gene product encoded by the ORF sequence of SEQ ID NO. 62 which is derived from the enzyme gene sequence of SEQ ID NO. 61. As used herein the terms “polygalacturonase 1”, “polygalacturonase 2” and “polygalacturonase 3” encompasses respectively, not only the polypeptides of SEQ ID NO: 57, 60, and 63, but also all the enzyme gene products related to SEQ ID NO: 55, 56, 58, 59, 61, and 62 as described above in section 5.2, including but not limited to homologs, splice variants, polypeptide fragments, fusion proteins, and functional derivatives, that display polygalacturonase activity. In preferred embodiments, homologs of polygalacturonase 1 having greater than 69% amino acid sequence identity with polygalacturonase 1, homologs of polygalacturonase 2 having greater than 80% amino acid sequence identity with polygalacturonase 2, and homologs of polygalacturonase 3 having greater than 80% amino acid sequence identity with polygalacturonase 3, are provided. Enzymes having polygalacturonase activity hydrolyses the glycosidic linkages in a polygalacturonic acid chain which are commonly found in plant cell walls. They exist mainly as chains of 1,4-linked a-D-galacturonic acid and methoxylated derivatives thereof. Accordingly, A. fumigatus polygalacturonases can be used to reduce the amounts of polygalacturonic acid polymers in a composition, or to produce monogalacturonic acid or galacturonic acid containing oligosaccharides from pectin-containing materials.

The enzymes of the invention are useful in the food industry, primarily in fruit and vegetable processing such as fruit juice production or wine making. For example, A. fumigatus polygalacturonase 1, polygalacturonase 2, and/or polygalacturonase 3 can be used in methods for degrading pectin polymers in plant-derived materials, e.g. obtained from soy beans, sugar beets, apples or pears, so as to reduce the viscosity and thus improve the processing or storage properties of the materials. The enzymes may also be used in the treatment of mash or pulp from fruits and vegetables in order to improve the properties of the mash for processing or disposal. For example, the consistency and appearance of processed fruit or vegetables can be manipulated with the polygalcturonases of the invention. The polygalacturonases of the invention can alone or together with other enzymes be used to improve the digestibility of pectin-containing animal feed. For example, the polygalacturonase 1, polygalacturonase 2, and/or polygalacturonase 3 of the invention can be used in the type of processes described in U.S. Pat. No. 5,830,737 and 6,159,718.

The invention further encompasses an enzyme composition comprising the polygalacturonase 1, polygalacturonase 2 and/or polygalacturonase 3, in free form or in an immobilized form. The invention further encompasses an eznyme composition comprising polygalacturonase 1, polygalacturonase 2 and/or polygalacturonase 3, and cellulases, xylanases, proteases, and pectin degrading enzymes, such as but not limited to a pectin methyl esterase, a pectin lyase, pectin acetyl esterase, a rhamnogalacturonase, a galactanase, an arabinanase and/or a rharnnogalacturonan acetyl esterase.

5.5.11. Xylanases

The present invention encompasses polypeptides having xylanase activity. The amino acid sequence of a first polypeptide of the invention having xylanase activity is set forth in SEQ ID NO. 66. The polypeptide of SEQ ID NO: 66, herein referred to as xylanase 1, is a gene product encoded by the ORF sequence of SEQ ID NO: 65 which is derived from the enzyme gene sequence of SEQ ID NO. 64. The amino acid sequence of a second polypeptide of the invention having xylanase activity is set forth in SEQ ID NO. 69. The polypeptide of SEQ ID NO: 69, herein referred to as xylanase 2, is a gene product encoded by the ORF sequence of SEQ ID NO. 68 which is derived from the enzyme gene sequence of SEQ ID NO. 67. The amino acid sequence of a third polypeptide of the invention having xylanase activity is set forth in SEQ ID NO. 72. The polypeptide of SEQ ID NO: 72, herein referred to as xylanase 3, is a gene product encoded by the ORF sequence of SEQ ID NO. 71 which is derived from the enzyme gene sequence of SEQ ID NO. 70. As used herein the terms “xylanase 1”, “xylanase 2” and “xylanase 3” encompasses respectively, not only the polypeptides of SEQ ID NO: 66, 69, and 72, but also all the enzyme gene products related to SEQ ID NO: 64, 65, 67, 68, 70, and 71 as described above in section 5.2, including but not limited to homologs, splice variants., polypeptide fragments, fusion proteins, and functional derivatives, that display xylanase activity. In preferred embodiments, homologs of xylanase 1 having greater than 73% amino acid sequence identity with xylanase 1, homologs of xylanase 2 having greater than 77% amino acid sequence identity with xylanase 2, and homologs of xylanase 3 having greater than 79% amino acid sequence identity with xylanase 3, are provided.

Xylan, a major component of plant hemicellulose, is a polymer of D-xylose linked by β-1,4-xylosidic bonds. Xylan can be degraded to xylose and xylo-oligomers by xylanases (EC3.2.1.8) that randomly cleave the β,1-4 linkages. When this plant cell wall polysaccharide is hydrolyzed with xylanases, it can be exploited as a rich source of carbon and energy for the production of livestock and microorganisms. Accordingly, A. fumigatus xylanase 1, xylanase 2, and/or xylanase 3 can be used in methods for degrading xylan, or methods for producing xylose and xylo-oligomers which may serve as growth substrates for microorganisms in various fermentation processes.

The A. fumigatus xylanase 1, xylanase 2, and/or xylanase 3 can also be used as an animal feed additive. The treatment of forages with xylanases along with cellulases increase the rate of acid production, thus ensuring better quality silage and improvement in the subsequent rate of plant cell wall digestion by ruminants. The xylanases can also be used to treat rye, and other cereals with a high arabinoxylan content to improve the digestibility of cereal by poultry and swine.

Enzymatic disruption of plant cell walls can increase the efficiency of a number of industrial processes. For example, the xylanases of the invention can be used in biopulping to treat cellulose pulps to remove xylan impurities or to produce pulps with different characteristics. Further, xylanases of the invention can be useful in the retting of flax fibers, the clarification of fruit juices, the preparation of dextrans for use as food thickeners and the production of fluids and juices from plant materials. For example, the xylanase 1, xylanase 2, and/or xylanase 3 of the invention can be used in the type of processes described in U.S. Pat. No. WO 91/19782, EP 463 706, WO 92/01793, and WO 92/17573. The invention further encompasses an enzyme composition comprising the xylanase 1, xylanase 2 and/or xylanase 3, in free form or in an immobilized form. The invention further encompasses an enzyme composition comprising the xylanase 1, xylanase 2 and/or xylanase 3, and cellulases, and hemicellulases.

### 6. EXAMPLES

Described below are techniques used in the analysis of genomic DNA from Aspergillus fumigatus and the cloning and expression of the enzyme genes of the invention.

6.1. Isolation of Genomic DNA from Aspergillus fumigatus

Genomic DNA was isolated from Aspergillus fumigatus strain CEA17 using a commercially available isolation kit (DNEasy Plant Mini Kit, Qiagen, Inc.) according to the manufacturer's instructions with the following minor modifications. Briefly, mycelia were cultured by collecting spores from a confluent plate using a wet inoculating loop and the scraped spores touched to the surface of culture medium placed in a 24 well culture dish. The spores were swirled in the medium to ensure even growth and the dish was incubated without shaking for about 14 to 16 hours at 37° C. The mycelia grow on the surface at the air-medium interface.

The mycelia were harvested using a sterile toothpick and placed between sterile paper towels. The mycelia were squeezed to remove excess liquid and the harvested mycelia were allowed to dry for 5-10 minutes. The semi-dry mycelia were placed into Bio101 Homogenizing Matrix tubes using a sterile toothpick. To each tube, 400 μl of lysis buffer (Buffer AP1) was added and the tubes were placed into the Bio101 FastPrep Apparatus (Qbiogene), run at a speed setting of 5 for 30 seconds, and then subjected to centrifugation in a nicrofuge for two minutes at maximum speed at 4° C.

The supernatant containing the genomic DNA was transferred to a sterile 1.5 ml tube, 4 μl of 100 mg/mL solution of RNase was added to each tube, and the tubes were incubated for 10 minutes at 65° C. Approximately 130 μl of protein precipitation buffer (Buffer AP2) was added, the tubes mixed and incubated for about 5 minutes on ice. The supernatant was applied to the supplied QIAshredder spin column (lilac) sitting in a 2 ml collection tube and subjected to centrifugation in a microfuge for 2 min at maximum speed. The flow-through fraction was transferred to a sterile tube without disturbing the cell-debris pellet, 0.5-volume of DNA precipitation buffer (Buffer AP3) and 1 volume of ethanol (96-100%) were added to the cleared supematant and the tubes mixed by inverting a couple times. The supematant was applied in 650 μl aliquots, including any precipitate that may have formed, to the supplied DNeasy mini-spin column sitting in a 2 ml collection tube (supplied). The column was subjected to centrifugation in a microfuge for 1 minute at >8000 rpm and flow-through and the collection tube were discarded. The DNEasy column was placed in the supplied 2 ml collection tube, 500 μl of wash buffer (Buffer AW) was added and the DNeasy column was subjected to centrifugation in a microfuge at >8000 rpm for about 1 minute. The flow-through was discarded and the genomic DNA was eluted twice by the addition of 100 μl of a preheated (56° C.-65° C.) elution buffer (Buffer AE). The above-described protocol typically results in ˜50-100 ng of genomic DNA/μl (approximately 200 μl elution volume).

6.2. Transformation of Aspergillus fumigatus Protoplasts

6.2.1. Growth and Harvest of Mycelia

An aliquot of approximate 10⁹ spores of Aspergillus fumigatus CEA17 was inoculated into 250 ml of non-selective medium supplemented with uridine and uracil, e.g., Aspergillus complete medium (ACM), and the culture was incubated with shaking at 250 rpm for about 14 to 16 hours at 30° C. After incubation, the culture is checked under a microscope to determine whether balls of mycelia have formed. If balls of mycelia are not evident, the culture was shifted to 37° C. and incubated for another 2-3 hours to stimulate mycelia ball formation. Approximately 10 transformation procedures can be performed from 250 ml of primary culture.

The mycelia were collected by filtration using a vacuum flask adapted with a sterile, cheesecloth-lined funnel. The collected mycelia were washed with 25 ml of a sterile solution of cold 0.6 M MgSO₄ and the washed mycelia were allowed to dry for about one minute. The mycelia were harvested using a sterile spatula to remove the mycelia from the cheesecloth and placed in a tube. The mass of mycelia should optimally occupy no more than 20% of the volume of the tube for optimal protoplast formation.

6.2.2. Generation and Collection of Protoplasts

Approximately a 10 ml volume of collected mycelia was placed in a 50 ml conical tube, and a sterile solution of osmotic medium (1.2 M MgSO₄, 10 mM NaPO₄, pH 5.8) is added to the tube to a final volume of 50 ml. The mycelia were dispersed by vortexing for 0.5 to 1 minute. In a separate 2 ml tube, 250 mg of Driselase enzyme (Interspex Products, San Mateo, Calif.) was added to about 1 ml of osmotic medium and placed on ice for 5 minutes. The tube was subjected to brief centrifugation at 14,000×G for 30 seconds to pellet the enzyme starch carrier. Failure to remove the starch carrier may interfere with obtaining protoplasts. The enzyme supematant was transferred to a sterile tube and 400 mg β-D-glucanase (Interspex Products, San Mateo, Calif.) was added. The enzyme mixture was allowed to dissolve, added to the 50 ml mycelia preparation, and mixed by inverting.

The contents of the tube were poured into 500 ml Erlenmeyer flask and incubated with shaking between 100-125 rpm for 2.5 hours at 30° C. The progress of protoplast formation was examined microscopically at various time intervals until complete. Protoplast formation is typically complete within two hours. The protoplast suspension was dispensed into several 50 ml conical tubes adding no more than 10 ml volume to each tube. The suspension was gently overlaid with an equal volume of sterile Trapping Buffer (0.6 M Sorbitol in 0.1 M Tris-Cl, pH 7.0) being careful not to mix the two layers. The tubes were subjected to centrifugation at 3,000×G in a swinging bucket rotor for 15 minutes. The fuzzy white layer of that forms at the Osmotic medium/Trapping Buffer interface containing the protoplasts was removed using a transfer pipette and the samples were combined.

The combined samples were placed into a plastic centrifuge tube capable of withstanding up to 10,000×g and an equal volume of sterile STC buffer (1.2 M sorbitol, 10 mM CaCl₂ in 10 mM Tris-HCl, pH 7) was added. The protoplasts were pelleted by subjecting the protoplast sample to centrifugation at 8,000×g for 8 minutes at 4° C. The supernatant of the sample was removed taking care not to disturb the pellet. The pellet was gently resuspended in 5 ml STC buffer using a transfer pipette and the protoplasts were pelleted by subjecting the protoplast sample to centrifugation at 8,000×g for 8 minutes at 4° C. The above-described STC buffer wash steps were repeated an additional two times, the protoplasts were combined into a single tube, and resuspended into an appropriate volume for transformation (approximately 100 μl protoplast suspension/transformation reaction).

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications and patents are cited throughout the specification. The disclosures of each of these publications and patents are incorporated by reference in its entirety.

What is claimed is:

1. An isolated nucleic acid molecule comprising (a) a nucleotide sequence that encodes a polypeptide, said polypeptide comprising an amino acid sequence selected from the group consisting of SEQ ID NO: 3, 6, 9, 12, 15, 18, 21, 24, 27, 30, 33, 36, 39, 42, 45, 48, 51, 54, 57, 60, 63, 66, 69, and 72; or (b) a complement of (a).

2. An isolated nucleic acid molecule comprising (a) a nucleotide sequence selected from the group consisting of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70; or (b) a complement of (a).

3. An isolated nucleic acid molecule comprising a nucleotide sequence that hybridizes under medium stringency conditions to a nucleic acid probe consisting of:
(a) a nucleotide sequence selected from the group consisting of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, 70, 73, 76, and 79;
(b) a nucleotide sequence that encodes a polypeptide consisting of an amino acid sequence selected from the group consisting of SEQ ID NO: 3, 6, 9, 12, 15, 18, 21, 24, 27, 30, 33, 36, 39, 42, 45, 48, 51, 54, 57, 60, 63, 66, 69, and 72; or
(c) the complement of the nucleotide sequence of (a), or (b);
wherein said medium stringency conditions comprise hybridization to filter-bound DNA in 6×sodium chloride/sodium citrate (SSC) at about 45° C. followed by one or more washes in 0.2×SSC/0.1% SDS at about 50 to about 65° C.

4. The isolated nucleic acid molecule of claim 1, 2, or 3, which is genomic DNA.

5. The isolated nucleic acid molecule of claim 1, 2, or 3, which is cDNA.

6. The isolated nucleic acid molecule of claim 1, 2, or 3, which is RNA.

7. The isolated nucleic acid molecule of claim 1, 2, or 3, which is single-stranded.

8. An isolated nucleic acid molecule comprising at least 8 consecutive nucleotides of:
(a) a nucleotide sequence that encodes a polypeptide, said polypeptide consisting of an amino acid sequence selected from the group consisting of SEQ ID NO: 3, 6, 9, 12, 15, 18, 21, 24, 27, 30, 33, 36, 39, 42, 45, 48, 51, 54, 57, 60, 63, 66, 69, and 72;
(b) a nucleotide sequence selected from the group consisting of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70; or
(c) the complement of the nucleotide sequence of (a), or (b).

9. A nucleic acid probe consisting of at least 8 nucleotides, wherein the nucleic acid probe is hybridizable under medium stringency conditions to at least a portion of:
(a) a nucleotide sequence selected from the group consisting of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70; or
(b) the complement of the nucleotide sequence of (a)
wherein said medium stringency conditions comprise hybridization to filter-bound DNA in 6×sodium chloride/sodium citrate (SSC) at about 45° C. followed by one or more washes in 0.2×SSC/0.1% SDS at about 50 to 65° C.

10. A nucleic acid molecule comprising a nucleotide sequence of claim 1, 2, or 3 uninterrupted by stop codons within a coding sequence that encodes a heterologous protein or peptide.

11. A recombinant vector comprising the nucleic acid molecule of claim 1, 2, 3, 8, or 10.

12. An expression construct comprising the nucleic acid molecule of claim 1, 2, 3, 8, or 10, wherein the nucleotide sequence is operatively associated with a regulatory nucleotide sequence containing transcriptional and/or translational regulatory signals that controls expression of the nucleotide sequence in a host cell.

13. A genetically engineered host cell comprising the nucleic acid molecule of claim 1, 2, 3, 6, 7, 8, or 10.

14. A genetically engineered host cell comprising the nucleic acid molecule of claim 1, 2, 3, 8, or 10, wherein the nucleotide sequence is operatively associated with a regulatory nucleotide sequence containing transcriptional and/or translational regulatory information that controls expression of the nucleotide sequence in the host cell.

15. A method for detecting in a sample the presence of a nucleic acid molecule that encodes an enzyme, said method comprising:
(a) contacting the sample with a nucleic acid probe of claim 9 under hybridizing conditions; and
(b) measuring the hybridization of the probe to the nucleic acids of the sample,
thereby detecting the presence of the nucleic acid molecule.

16. A method of making a polypeptide comprising the steps of:
(a) culturing the cell of claim 14 under the appropriate conditions to produce the polypeptide; and
(b) isolating the polypeptide.

17. An isolated polypeptide comprising an amino acid sequence selected from the group consisting of SEQ ID NO: 3, 6, 9, 12, 15, 18, 21, 24, 27, 30, 33, 36, 39, 42, 45, 48, 51, 54, 57, 60, 63, 66, 69, 72, 75, 78, and 81.

18. An isolated polypeptide comprising an amino acid sequence encoded by a nucleotide sequence selected from the group consisting of SEQ ID NO: 2, 5, 8, 11, 14, 17, 20, 23, 26, 29, 32, 35, 38, 41, 44, 47, 50, 53, 56, 59, 62, 65, 68, 71, 1, 4, 7, 10, 13, 16, 19, 22, 25, 28, 31, 34, 37, 40, 43, 46, 49, 52, 55, 58, 61, 64, 67, and 70.

19. An isolated polypeptide, the amino acid sequence of which comprises at least six consecutive residues of an amino acid sequence selected from the group consisting of SEQ ID NO: 3, 6, 9, 12, 15, 18, 21, 24, 27, 30, 33, 36, 39, 42, 45, 48, 51, 54, 57, 60, 63, 66, 69, and 72.

20. An isolated polypeptide, the amino acid sequence of which is selected from the group consisting of SEQ ID NO: 3, 6, 9, 12, 15, 18, 21, 24, 27, 30, 33, 36, 39, 42, 45, 48, 51, 54, 57, 60, 63, 66, 69, and 72 with at least one conservative amino acid substitution.

21. An isolated polypeptide comprising an amino acid sequence which is at least 60% identical to an amino acid sequence selected from the group consisting of SEQ ID NO: 3, 6, 9, 12, 15, 18, 21, 24, 27, 30, 33, 36, 39, 42, 45, 48, 51, 54, 57, 60, 63, 66, 69, and 72, and displays the catalytic activity of an enzyme selected from the group consisting of tannase, cellulase, glucose oxidase, glucoamylase, phytase, β-galactosidases, invertase, lipase, α-amylase, laccase, polygalacturonase and xylanase.

22. A chimeric protein comprising a polypeptide of claim 19 fused via a covalent bond to an amino acid sequence of a second polypeptide.

23. An enzymatic composition comprising the polypeptide of claim 17, 18, or 21.

24. The enzymatic composition of claim 23, wherein the composition is in solid form.

25. The enzymatic composition of claim 23 further comprising one or more solid phase, wherein the polypeptide is present on the solid phase.

26. An enzymatic composition comprising (a) a lysate of the genetically engineered cells of claim 14, or (b) a culture medium in which the genetically engineered cells of claim 14 were cultured.

27. An enzymatic composition enriched for a polypeptide which is at least 60% identical to the polypeptide, the amino acid sequence of which is selected from the group consisting of SEQ ID NO: 3, 6, 9, 12, 15, 18, 21, 24, 27, 30, 33, 36, 39, 42, 45, 48, 51, 54, 57, 60, 63, 66, 69, and 72, and displays the catalytic activity of an enzyme selected from the group consisting of tannase, cellulase, glucose oxidase, glucoamylase, phytase, β-galactosidases, invertase, lipase, α-amylase, laccase, polygalacturonase and xylanase.

28. An antibody preparation which binds to the polypeptide of claim 17.

29. A molecule comprising a fragment of the antibody of claim 28, which fragment binds to the polypeptide of claim 17

30. The antibody preparation of claim 28 which comprises a monoclonal antibody.

31. A kit comprising in one or more containers the enzymatic composition of claim 23.

32. A method for modulating the amount of compounds that comprise a gallate ester linkage in a composition comprising contacting the composition with an enzymatic composition, wherein the enzymatic composition comprises (a) a polypeptide comprising an amino acid sequence selected from the group consisting of SEQ ID NO: 3 and 6, (b) a polypeptide encoded by a nucleotide sequence selected from the group consisting of SEQ ID NO: 1, 2, 4, and 5; or (c) a polypeptide comprising an amino acid sequence which is at least 60% identical to the amino acid sequence of the polypeptide of (a) and displays the catalytic activity of a tannase.

33. A method for modulating the amount of cellulose in a composition comprising contacting the composition with an enzymatic composition, wherein the enzymatic composition comprises (a) a polypeptide comprising the amino acid sequence of SEQ ID NO: 9, (b) a polypeptide encoded by a nucleotide sequence selected from the group consisting of SEQ ID NO: 7 and 8, or (c) a polypeptide comprising an amino acid sequence which is at least 60% identical to the amino acid sequence of the polypeptide of (a) and displays the catalytic activity of a cellulase.

34. A method for modulating the amount of glucose or oxygen in a composition comprising contacting the composition with an enzymatic composition, wherein the enzymatic composition comprises (a) a polypeptide comprising an amino acid sequence selected from the group consisting of SEQ ID NO: 12, 15, and 18, (b) a polypeptide encoded by a nucleotide sequence selected from the group consisting of SEQ ID NO: 10, 11, 13, 14, 16, and 17, or (c) a polypeptide comprising an amino acid sequence which is at least 60% identical to the amino acid sequence of the polypeptide of (a) and displays the catalytic activity of a glucose oxidase.

35. A method for modulating the amount of myo-inositol phosphates in a composition comprising contacting the composition with an enzymatic composition, wherein the enzymatic composition comprises (a) a polypeptide comprising the amino acid sequence of SEQ ID NO: 24, (b) a polypeptide encoded by a nucleotide sequence selected from the group consisting of SEQ ID NO: 22 and 23, or (c) a polypeptide comprising an amino acid sequence which is at least 60% identical to the amino acid sequence of the polypeptide of (a) and displays the catalytic activity of a phytase.

36. A method for modulating the amount of lactose in a composition comprising contacting the composition with an enzymatic composition, wherein the enzymatic composition comprises (a) a polypeptide comprising an amino acid sequence selected from the group consisting of SEQ ID NO: 27 and 30, (b) a polypeptide encoded by a nucleotide sequence selected from the group consisting of SEQ ID NO: 25, 26, 28, and 29, or (c) a polypeptide comprising an amino acid sequence which is at least 60% identical to the amino acid sequence of the polypeptide of (a) and displays the catalytic activity of a β-galactosidase.

37. A method for modulating the amount of sucrose in a composition comprising contacting the composition with an enzymatic composition, wherein the enzymatic composition comprises (a) a polypeptide comprising the amino acid sequence of SEQ ID NO: 36, (b) a polypeptide encoded by a nucleotide sequence selected from the group consisting of SEQ ID NO: 34 and 35, or (c) a polypeptide comprising an amino acid sequence which is at least 60% identical to the amino acid sequence of the polypeptide of (a) and displays the catalytic activity of an invertase.

38. A method for modulating the amount of glyceride in a composition comprising contacting the composition with an enzymatic composition, wherein the enzymatic composition comprises (a) a polypeptide comprising the amino acid sequence of SEQ ID NO: 39, (b) a polypeptide encoded by a nucleotide sequence selected from the group consisting of SEQ ID NO: 37 and 38, or (c) a polypeptide comprising an amino acid sequence which is at least 60% identical to the amino acid sequence of the polypeptide of (a) and displays the catalytic activity of a lipase.

39. A method for modulating the amount of starches or maltodextrins in a composition comprising contacting the composition with an enzymatic composition, wherein the enzymatic composition comprises (a) a polypeptide comprising an amino acid sequence selected from the group consisting of SEQ ID NO: 42, 45, and 48, (b) a polypeptide encoded by a nucleotide sequence selected from the group consisting of SEQ ID NO: 40, 41, 43, 44, 46 and 47; or (c) a polypeptide comprising an amino acid sequence which is at least 60% identical to the amino acid sequence of the polypeptide of (a) and displays the catalytic activity of an α-amylase.

40. A method for modulating the amount of oxidated phenolic compounds in a composition comprising contacting the composition with an enzymatic composition, wherein the enzymatic composition comprises (a) a polypeptide comprising the amino acid sequence of SEQ ID NO: 51, (b) a polypeptide encoded by a nucleotide sequence selected from the group consisting of SEQ ID NO: 49 and 50; or (c) a polypeptide comprising an amino acid sequence which is at least 60% identical to the amino acid sequence of the polypeptide of (a) and displays the catalytic activity of a laccase.

41. A method for modulating the amount of high molecular weight polygalacturonic acid chains or low molecular weight polygalacturonic acid chains in a composition comprising contacting the composition with an enzymatic composition, wherein the enzymatic composition comprises (a) a polypeptide comprising an amino acid sequence selected from the group consisting of SEQ ID NO: 57, 60 and 63, (b) a polypeptide encoded by a nucleotide sequence selected from the group consisting of SEQ ID NO: 55, 56, 58, 59, 61 and 62; or (c) a polypeptide comprising an amino acid sequence which is at least 60% identical to the amino acid sequence of the polypeptide of (a) and displays the catalytic activity of a polygalacturonase.

42. A method for modulating the amount of xylan or xylo-oligomers in a composition comprising contacting the composition with an enzymatic composition, wherein the enzymatic composition comprises (a) a polypeptide comprising an amino acid sequence selected from the group consisting of SEQ ID NO: 66, 69, and 72; (b) a polypeptide encoded by a nucleotide sequence selected from the group consisting of SEQ ID NO: 64, 65, 67, 68, 70, and 71; or (c) a polypeptide comprising an amino acid sequence which is at least 60% identical to the amino acid sequence of the polypeptide of (a) and displays the catalytic activity of a xylanase.

43. A method for identifying a compound that modulates the activity of an enzyme, comprising:
(a) contacting a test compound with an enzymatic composition of claim 23; and
(b) detecting a change in the activity of the enzyme as compared to an enzymatic composition that is not contacted with the test compound;
wherein the activity of the enzyme is that of an enzyme selected from the group consisting of tannase, cellulase, glucose oxidase, glucoamylase, phytase, β-galactosidases, invertase, lipase, α-amylase, laccase, polygalacturonase and xylanase.

44. A method for identifying an enzyme with modified chemical and/or physical characteristics, comprising:
(a) mutagenizing a nucleotide sequence that encodes the enzyme; and
(b) determining the chemical and/or physical characteristics of the enzyme produced as a result of expression of the mutagenized nucleotide sequence.

45. The method of claim 44 wherein the mutagenizing step comprises using portions of the nucleotide sequence of another enzyme that is imperfectly matched to the nucleotide sequence of the enzyme in a sequence alignment.


SEQUENCE LISTING (72 sequences)

SEQ ID NO: 1 - DNA, 1722 bp, Aspergillus
atgcgtccctctagctgcatccccttctccgtgggccttgccgttgtggcccatgcagcgtctccgtcaaccctggaggagctctgcaccgtttcatatcttcaaactgtgttgccttcatctaagttcattcagggcatcaccattgactcagactctctcacaaccagtgtggtgaccaacagcactgtcagcagcgttgactaccctacggcgacgatcgactactgcaatgtcaccctggcctactcgcacgatggtatcgacggcgaccgagtcctactccaaatatggctaccagctcctaccgacttccaaaaccgatggctgtccaccggaggcggtggttatgccatcaattccgggacgcgaatgctccctgagggtatcatctacggtgcggcgtctggactcacggacggtgggtttgggggcttctcggtgaatgccgacagcgctatgttgctggccaacggtacactgaactatgaggcgctgtacatgttcgggtacaaggctcaccgggaacttagcttgattggcaaggccttcacgcggaaggtctacggcatggccgacagtgagaagctctatgcctactaccatggatgctccgagggtggccgtgagggctggagccaagtgcagcgctacggtgacgaatgggatggggccattatcggggctccagcgtttcgttggtcgtttcagcagactcaacatctgttctcgaatatcgttgagaagactcttgactactaccctccgccctgcgagctggagaagattgtgaacgagactatcgttgcatgcgatcccttggacggcaggactgatggtgtggtcgctcgaaccgatctctgcctgctgcatttcgacctgaaacatgtcattggcaagaagtactcctgcgcggcttctaccaccgcaccggcacagagtggcactgtttcagccaaggcagtcgaggtcgctaaaaccatcattaacggactgcatgacactcagggccgtcgagtgtacttctcctaccaacccagtgccgcattcgacgatgctcagacgcagttcaatgccgacaccggcaagtgggagctgtctatcaaccaactcgggggcaaacacattgcactgctgatgaacaaaaacagcaccaccctggacagcctaaacggcgttacctacgacacgttgaaagactggataatttcgggcatgcaggagtactacagcaccctgcagactacctggcccgatctcacacccttccaccaggctggcggcaaagtgatccactaccatggcgacgctgatttcagcatccccactgcttcgtctatccgctactgggagtcggtgcggagtaccatgtacggcaacctgtcgtacaaagctggtgctaacgcattgaatgaatggtaccgcttatacactgtgcctggtgccggccactgctcgaccaacgatgccatgcccaatggtccatggcctcagaccaacctcgctaccatggtagaatgggtcgaaaagggagtgactccggtcaccctgaatgcaacagtgctccagggcgagtatgaaggcgagacccagcagctctgcgcgtggccattgcgtcctctctggaagaacaaaggaaagaccctaaactgtgtgtatgaccaggcgtccatcaacagctggcactatgacttggatgcagttcctatgcctgtatattag

SEQ ID NO: 2 - DNA, 1722 bp, Aspergillus; identical to SEQ ID NO: 1
  CDS (1)...(1722)

SEQ ID NO: 3 - protein, 573 aa, Aspergillus
MRPSSCIPFSVGLAVVAHAASPSTLEELCTVSYLQTVLPSSKFIQGITIDSDSLTTSVVTNSTVSSVDYPTATIDYCNVTLAYSHDGIDGDRVLLQIWLPAPTDFQNRWLSTGGGGYAINSGTRMLPEGIIYGAASGLTDGGFGGFSVNADSAMLLANGTLNYEALYMFGYKAHRELSLIGKAFTRKVYGMADSEKLYAYYHGCSEGGREGWSQVQRYGDEWDGAIIGAPAFRWSFQQTQHLFSNIVEKTLDYYPPPCELEKIVNETIVACDPLDGRTDGVVARTDLCLLHFDLKHVIGKKYSCAASTTAPAQSGTVSAKAVEVAKTIINGLHDTQGRRVYFSYQPSAAFDDAQTQFNADTGKWELSINQLGGKHIALLMNKNSTTLDSLNGVTYDTLKDWIISGMQEYYSTLQTTWPDLTPFHQAGGKVIHYHGDADFSIPTASSIRYWESVRSTMYGNLSYKAGANALNEWYRLYTVPGAGHCSTNDAMPNGPWPQTNLATMVEWVEKGVTPVTLNATVLQGEYEGETQQLCAWPLRPLWKNKGKTLNCVYDQASINSWHYDLDAVPMPVY

SEQ ID NO: 4 - DNA, 1767 bp, Aspergillus
atgcgtatctcctacggctcagccgttgctgctctggcagcggcagctaatgctgcatctctcgctgacgtgtgcaccatctcccatgtgcagtccgtgcttccttcaaacggaactcttctgggtatcaacgtgattccgtccgccgtgactgcaagtgcagtctacaacagtacctccagcggcggcatgggcggcatgggcggctccaacagtgccaactacccctactgcaatgtgacggtcacctatacccaccccggtaagggcgacaaggtggtcgttaagtatgccttcccccagccttccgacttcaagaaccgcttctacgttgccgggggtggcggttactccctctccagcgatgccactggcggtctagagtatggtgcggcgtctggtgccaccgatgcgggttacgatgccttcagctacagctacgacgaggtggttctttacggcaacggatcgatcaactgggacgccacgtacatgttcgcctaccaggccctgggcgagatgaccactctcggcaagacattgacccgaaacttctacggtctgtctagcgatgccaagatctacacctactacgaaggatgctccgacggtggacgtgaaggcatgagccaggttcagcgctacggcgatctgtacgacggtgccatcaccggtgccccggctttccgctatgcccagcagcaggtccaccacgtcttttcgtcggttgtggagaagactctggactactaccctcccccgtgcgaactggccaagatcgtcaacgccaccattgaggcctgcgatcctctcgacggccgcactgacggggtggtctcccgcaccgacctctgcaagctgcactttgacctttcgaagatcatcggggagccgtactactgcgccgcaaagaccagcacctccctcggcttcggcttcagcaaacgccaggcagccggcagcacgaccagctaccagcctgcacaaaacggcaccgtcaccaaggagggggtggctgtcgccaaggccatttacgacggtctgcacaacacccagggccagcgcgcctacctctcctggcagatcgcctcggaattctccgatgccaccaccgagtggaacaatgacaccggctcctgggagctgagcatcccgtccaccggcggcgaattcgtgaccaagttcgtccaactcctggatctcgataacctgtccactctcgacaacgtcacctacgacaccctcgtcgaatggatgaacaccgccatggtgcgctacatggacagcctgcagaccaccgtcccggacctgacgactttcaaatcctccggcggcaagctgctgcattaccacggtgaatccgaccccagtatccccgccgcgtcctcggtgcactactggcagtctgtgcgctcgatcatgtaccccggtgtgtcggccgcgaagagtctgaaggagctccaggaatggtaccagttctatctcatccccggtgcggcgcactgcggtgccaactccctgcagcctggcccgtacccgcagaacaacatggacatcatgatcgactgggtggagaacggcgtgcagccgagccgactgaacaccacagtgtcctcgggcgactacgccggcgagacccagatgctctgccagtggccgacccgtcctctgtggaaggacaactcgacctttgactgtgtcaacgacgaaaagtcgatcgagagctggacctatacctttcctgctttcaaggtatctgtctactga

SEQ ID NO: 5 - DNA, 1767 bp, Aspergillus; identical to SEQ ID NO: 4
  CDS (1)...(1767)

SEQ ID NO: 6 - protein, 588 aa, Aspergillus
MRISYGSAVAALAAAANAASLADVCTISHVQSVLPSNGTLLGINVIPSAVTASAVYNSTSSGGMGGMGGSNSANYPYCNVTVTYTHPGKGDKVVVKYAFPQPSDFKNRFYVAGGGGYSLSSDATGGLEYGAASGATDAGYDAFSYSYDEVVLYGNGSINWDATYMFAYQALGEMTTLGKTLTRNFYGLSSDAKIYTYYEGCSDGGREGMSQVQRYGDLYDGAITGAPAFRYAQQQVHHVFSSVVEKTLDYYPPPCELAKIVNATIEACDPLDGRTDGVVSRTDLCKLHFDLSKIIGEPYYCAAKTSTSLGFGFSKRQAAGSTTSYQPAQNGTVTKEGVAVAKAIYDGLHNTQGQRAYLSWQIASEFSDATTEWNNDTGSWELSIPSTGGEFVTKFVQLLDLDNLSTLDNVTYDTLVEWMNTAMVRYMDSLQTTVPDLTTFKSSGGKLLHYHGESDPSIPAASSVHYWQSVRSIMYPGVSAAKSLKELQEWYQFYLIPGAAHCGANSLQPGPYPQNNMDIMIDWVENGVQPSRLNTTVSSGDYAGETQMLCQWPTRPLWKDNSTFDCVNDEKSIESWTYTFPAFKVSVY

SEQ ID NO: 7 - DNA, 1713 bp, Aspergillus
atgaagcaccttgcatcttccatcgcattgactctactgttgcctgccgtgcaggcccagcagaccgtatggggccaatgtatgttctggctgtcactggaataagactgtatcaactgctgatatgcttctaggtggcggccaaggctggtctggcccgacgagctgtgttgccggcgcagcctgtagcacactgaatccctgtatgttagatatcgtcctgagtggagacttatactgacttccttagactacgctcagtgtatcccgggagccaccgcgacgtccaccaccctcacgacgacgacggcggcgacgacgacatcccagaccaccaccaaacctaccacgactggtccaactacatccgcacccaccgtgaccgcatccggtaaccctttcagcggctaccagctgtatgccaacccctactactcctccgaggtccatactctggccatgccttctctgcccagctcgctgcagcccaaggctagtgctgttgctgaagtgccctcatttgtttggctgtaagtggccttatcccaatactgagaccaactctctgacagtcgtagcgacgttgccgccaaggtgcccactatgggaacctacctggccgacattcaggccaagaacaaggccggcgccaaccctcctatcgctggtatcttcgtggtctacgacttgccggaccgtgactgcgccgctctggccagtaatggcgagtactcaattgccaacaacggtgtggccaactacaaggcgtacattgacgccatccgtgctcagctggtgaagtactctgacgttcacaccatcctcgtcatcggtaggccgtacacctccgttgcgcgccgcctttctctgacatcttgcagaacccgacagcttggccaacctggtgaccaacctcaacgtcgccaaatgcgccaatgcgcagagcgcctacctggagtgtgtcgactatgctctgaagcagctcaacctgcccaacgtcgccatgtacctcgacgcaggtatgcctcacttcccgcattctgtatcccttccagacactaactcatcaggccatgcgggctggctcggatggcccgccaacttgggccccgccgcaacactcttcgccaaagtctacaccgacgcgggttcccccgcggctgttcgtggcctggccaccaacgtcgccaactacaacgcctggtcgctcagtacctgcccctcctacacccagggagaccccaactgcgacgagaagaagtacatcaacgccatggcgcctcttctcaaggaagccggcttcgatgcccacttcatcatggatacctgtaagtgcttattccaatcgccgatgtgtgccgactaatcaatgtttcagcccggaatggcgtccagcccacgaagcaaaacgcctggggtgactggtgcaacgtcatcggcaccggcttcggtgttcgcccctcgactaacaccggcgatccgctccaggatgcctttgtatggatcaagcccggtggagagagtgatggcacgtccaactcgacttccccccggtatgacgcgcactgcggatatagtgatgctctgcagcctgctcctgaggctggtacttggttccaggtatgtcatccattagccagatgagggataagtgactgacggacctaggcctactttgagcagcttctgaccaacgctaacccgtccttttaa

SEQ ID NO: 8 - DNA, 1365 bp, Aspergillus
  CDS (1)...(1365)
atgaagcaccttgcatcttccatcgcattgactctactgttgcctgccgtgcaggcccagcagaccgtatggggccaatgtggcggccaaggctggtctggcccgacgagctgtgttgccggcgcagcctgtagcacactgaatccctactacgctcagtgtatcccgggagccaccgcgacgtccaccaccctcacgacgacgacggcggcgacgacgacatcccagaccaccaccaaacctaccacgactggtccaactacatccgcacccaccgtgaccgcatccggtaaccctttcagcggctaccagctgtatgccaacccctactactcctccgaggtccatactctggccatgccttctctgcccagctcgctgcagcccaaggctagtgctgttgctgaagtgccctcatttgtttggctcgacgttgccgccaaggtgcccactatgggaacctacctggccgacattcaggccaagaacaaggccggcgccaaccctcctatcgctggtatcttcgtggtctacgacttgccggaccgtgactgcgccgctctggccagtaatggcgagtactcaattgccaacaacggtgtggccaactacaaggcgtacattgacgccatccgtgctcagctggtgaagtactctgacgttcacaccatcctcgtcatcgaacccgacagcttggccaacctggtgaccaacctcaacgtcgccaaatgcgccaatgcgcagagcgcctacctggagtgtgtcgactatgctctgaagcagctcaacctgcccaacgtcgccatgtacctcgacgcaggccatgcgggctggctcggatggcccgccaacttgggccccgccgcaacactcttcgccaaagtctacaccgacgcgggttcccccgcggctgttcgtggcctggccaccaacgtcgccaactacaacgcctggtcgctcagtacctgcccctcctacacccagggagaccccaactgcgacgagaagaagtacatcaacgccatggcgcctcttctcaaggaagccggcttcgatgcccacttcatcatggatacctcccggaatggcgtccagcccacgaagcaaaacgcctggggtgactggtgcaacgtcatcggcaccggcttcggtgttcgcccctcgactaacaccggcgatccgctccaggatgcctttgtatggatcaagcccggtggagagagtgatggcacgtccaactcgacttccccccggtatgacgcgcactgcggatatagtgatgctctgcagcctgctcctgaggctggtacttggttccaggcctactttgagcagcttctgaccaacgctaacccgtccttttaa

SEQ ID NO: 9 - protein, 454 aa, Aspergillus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 10 - DNA, 1866 bp, Aspergillus
atgaccgaatacgactatatcattgtcggcgcaggcatcggtggcctggtgctggccaaccgcctgagcgcagacgcctctgtcaatgtccttctcatcgaagccggggccaatcgcatgggcgatcctcgtatcgacacccctgggtttctgggcatgttgtacggaaacccagacttcgactgggattatatgagtgtccctcaggtatcacctctcttccactgctccatttacggcaacaatgcaacaatgctgatcgtatagccgcatgtcaacaaccgacaaatcgcgcagccccgcggccgggtcgtcggcggttcctcggccctgaatttctcggtcgtcctgtaccctcccgcatcagacttcgaggcatggaaggcgttgggaaaccaaggctggggcgcagaggatatggcgccgtacctgcggaagtttcacacgttctccccgcccagcaagtccacggccgaccttctcggcgtcgatagctacatgaaagccagcagccaggggtgtgacggccctgtgcctgtttccctgccggacgtatatgggccgttcaacgaagcgtgggataagacgtttgagaagcttggttggcggacggacgcggatcccatcgccggacgcaaactgggtgcgttcaccccgccgctgaccgttgacgcaaagacagggaagcgggggtatgcagcggcctactactcccccgaggtggccgcacggccaaacctgcgcctgctggcagagaccatggtcgagcgggtgctgttgaccaggcaggatggagacgtgctggccaccggcgtgctggtcaaggacaaggatggcgcgagagagattcacgcaaagaaagaagtcattatctgcgccggcagcctgaatacgcctcagatcctggaactctccggcattggaaatgcagggctgctacagaagcacgacatccccgtggtgatcgacaaccccggcgttggcgagaacctccaagatcactgcatcagctgcatcagcttcgagatcgccgacggtcaagtctcgggcgacattctccgtgaccccaacgtggtacaggccctcgtcaagctgtacgaggagactcgcggcggcccgctggcaggcatgcccatcagcgtggcgtaccttcctttcgttgacggacgaggcgtcgtcccgcggccagaagtcgaggagctgctggccaagtatctcgacagcgccgcccttccacccaatctgcaggcgcagtacgcgcacctcaggaaagcgatcctcgacaacgataccccgtcgtcagagtacctcttcctccccgcgcagctacacatgaaacccggtgcgacaagcttgcccgacgtcctggccaagcccctgccggagaactacatcagcatcatgatcctgcacaaccaccctttctcacgaggatcggtgcatatctcctcccccaaggcggaggacaaaccgatctacgatccgaactttctctcccacccgctggacttggagatactcgctcggcacacgcaattccttgagacaatcgccgccacggagccgttcaagtctcttctcaaggaacggcggatcccggaaaacgcgagggacttgggcgatctggagcgagccaaggagctggtcaaggaccggctgtttacgtgctttcaccctgcggggacgtgtgccatgttgccgcgggagatggggggtgtggtggatgaccagctgagagtgtatgggacacgcaatctgagggtggttgatgccagtgtctttccgctggagccggcggggaatatccaggcgacggtgtatgcggtggcggagcgcgcagctgatttgatcatcggcagtaattag

SEQ ID NO: 11 - DNA, 1806 bp, Aspergillus
  CDS (1)...(1806)
atgaccgaatacgactatatcattgtcggcgcaggcatcggtggcctggtgctggccaaccgcctgagcgcagacgcctctgtcaatgtccttctcatcgaagccggggccaatcgcatgggcgatcctcgtatcgacacccctgggtttctgggcatgttgtacggaaacccagacttcgactgggattatatgagtgtccctcagccgcatgtcaacaaccgacaaatcgcgcagccccgcggccgggtcgtcggcggttcctcggccctgaatttctcggtcgtcctgtaccctcccgcatcagacttcgaggcatggaaggcgttgggaaaccaaggctggggcgcagaggatatggcgccgtacctgcggaagtttcacacgttctccccgcccagcaagtccacggccgaccttctcggcgtcgatagctacatgaaagccagcagccaggggtgtgacggccctgtgcctgtttccctgccggacgtatatgggccgttcaacgaagcgtgggataagacgtttgagaagcttggttggcggacggacgcggatcccatcgccggacgcaaactgggtgcgttcaccccgccgctgaccgttgacgcaaagacagggaagcgggggtatgcagcggcctactactcccccgaggtggccgcacggccaaacctgcgcctgctggcagagaccatggtcgagcgggtgctgttgaccaggcaggatggagacgtgctggccaccggcgtgctggtcaaggacaaggatggcgcgagagagattcacgcaaagaaagaagtcattatctgcgccggcagcctgaatacgcctcagatcctggaactctccggcattggaaatgcagggctgctacagaagcacgacatccccgtggtgatcgacaaccccggcgttggcgagaacctccaagatcactgcatcagctgcatcagcttcgagatcgccgacggtcaagtctcgggcgacattctccgtgaccccaacgtggtacaggccctcgtcaagctgtacgaggagactcgcggcggcccgctggcaggcatgcccatcagcgtggcgtaccttcctttcgttgacggacgaggcgtcgtcccgcggccagaagtcgaggagctgctggccaagtatctcgacagcgccgcccttccacccaatctgcaggcgcagtacgcgcacctcaggaaagcgatcctcgacaacgataccccgtcgtcagagtacctcttcctccccgcgcagctacacatgaaacccggtgcgacaagcttgcccgacgtcctggccaagcccctgccggagaactacatcagcatcatgatcctgcacaaccaccctttctcacgaggatcggtgcatatctcctcccccaaggcggaggacaaaccgatctacgatccgaactttctctcccacccgctggacttggagatactcgctcggcacacgcaattccttgagacaatcgccgccacggagccgttcaagtctcttctcaaggaacggcggatcccggaaaacgcgagggacttgggcgatctggagcgagccaaggagctggtcaaggaccggctgtttacgtgctttcaccctgcggggacgtgtgccatgttgccgcgggagatggggggtgtggtggatgaccagctgagagtgtatgggacacgcaatctgagggtggttgatgccagtgtctttccgctggagccggcggggaatatccaggcgacggtgtatgcggtggcggagcgcgcagctgatttgatcatcggcagtaattag

SEQ ID NO: 12 - protein, 601 aa, Aspergillus
MTEYDYIIVGAGIGGLVLANRLSADASVNVLLIEAGANRMGDPRIDTPGFLGMLYGNPDFDWDYMSVPQPHVNNRQIAQPRGRVVGGSSALNFSVVLYPPASDFEAWKALGNQGWGAEDMAPYLRKFHTFSPPSKSTADLLGVDSYMKASSQGCDGPVPVSLPDVYGPFNEAWDKTFEKLGWRTDADPIAGRKLGAFTPPLTVDAKTGKRGYAAAYYSPEVAARPNLRLLAETMVERVLLTRQDGDVLATGVLVKDKDGAREIHAKKEVIICAGSLNTPQILELSGIGNAGLLQKHDIPVVIDNPGVGENLQDHCISCISFEIADGQVSGDILRDPNVVQALVKLYEETRGGPLAGMPISVAYLPFVDGRGVVPRPEVEELLAKYLDSAALPPNLQAQYAHLRKAILDNDTPSSEYLFLPAQLHMKPGATSLPDVLAKPLPENYISIMILHNHPFSRGSVHISSPKAEDKPIYDPNFLSHPLDLEILARHTQFLETIAATEPFKSLLKERRIPENARDLGDLERAKELVKDRLFTCFHPAGTCAMLPREMGGVVDDQLRVYGTRNLRVVDASVFPLEPAGNIQATVYAVAERAADLIIGSN

SEQ ID NO: 13 - DNA, 1680 bp, Aspergillus
atgaagctccaacgcctcctctctatcaccctcacctttaccatcaccgcgccaacagccacagccacagcttcagcttcagctaaagcagatgcagaagcagaagcagactatctcgtcaccggtggcggcacaaccggcctcctcctcgccaaccgcctctcttcaacacccacgaccaccgttttgatcctcgaccccggcaacgacatccgcacgaaccccaatgtcaccgatccaacactctggctccgcaacgcacacacagaggtcgactgggcctacccctccaccccccagtcccacgccctcaaccgcatcctctcctacaccgccggccgcatcctaggcggcacgagcatgatcaacgggatgacgtacctgcgcgccgacaagcccgagatcgatgcgtgggaggcattaggtgctaaggggtggaattgggggagcttgtggccgtattatttgcgcacggagaagttcagtccgccgctgggatggcaggtgggtgctggggcggattatgtgcctgatttacatgggagaacggggagtgtggatgtgtgtttctcgatggagttgtcacgggtagggttttgggagagggtgagggatgcgtggagggttctaggggtgaattggaatagagatccgaatggggggtcggttgcaggggtgtcggtgtggccgcagacgattgattatcaggaggatgtgaggtgtagttctgcgaaggcgttttattatcctgttgaggggagggagaatctgagggttgtgaaggggaccgtcagaaggattctttgggctgacacccggggaggagagcatgtggcggcgggagtcgagtatctggatgagaacggacagatgcgaactgcaacggcccgcaaagaagtgatcctgtcagctggggcactgcggacaccccccatcctggaagcatcaggcgttggcgacgcagacaggctcagaggacttgggatagagacgcgaatcgatctccctggcgtaggagagaacctgcaagaccaggcaaacgtgcctctgctatacacgggaaacctcaatatctcagggacctcgccgtacgccacgttcctcatggcatcacagctctttggcgagaatctcgaagctgtagctgcgtaagttccctccgtctggcttgcacactggctgacatgccgcagagagacactctcctccgttccgtcctgggccgactccctcgcctcatcctcatccaacaacctcaacagcagcgcgatagaacgcctcctcaccctccagcacgccctcatcttcaaatccaacgtcactatagcagaaattctcaccagcgcgtccggaaccaccctcctctctgccttctggctcctcctccctttctcccggggcagcgtgcacctctcctcaacaaggcgagaagatataaacgcacccagcattgacacaaatttcttccaggtggactttgatctccagaccgagatggccatagggagactagcgcagtcattctgggaacaggggcccgtgaagagtctacatcccgttccgatgcccggcagggcattggatgataatgcaacagacacagaatggacagcgtttactaaagaaacctgtgagttgcatctggcagagaaaaatgaagaaaaggggcatactgattga

SEQ ID NO: 14 - DNA, 1635 bp, Aspergillus
  CDS (1)...(1635)
atgaagctccaacgcctcctctctatcaccctcacctttaccatcaccgcgccaacagccacagccacagcttcagcttcagctaaagcagatgcagaagcagaagcagactatctcgtcaccggtggcggcacaaccggcctcctcctcgccaaccgcctctcttcaacacccacgaccaccgttttgatcctcgaccccggcaacgacatccgcacgaaccccaatgtcaccgatccaacactctggctccgcaacgcacacacagaggtcgactgggcctacccctccaccccccagtcccacgccctcaaccgcatcctctcctacaccgccggccgcatcctaggcggcacgagcatgatcaacgggatgacgtacctgcgcgccgacaagcccgagatcgatgcgtgggaggcattaggtgctaaggggtggaattgggggagcttgtggccgtattatttgcgcacggagaagttcagtccgccgctgggatggcaggtgggtgctggggcggattatgtgcctgatttacatgggagaacggggagtgtggatgtgtgtttctcgatggagttgtcacgggtagggttttgggagagggtgagggatgcgtggagggttctaggggtgaattggaatagagatccgaatggggggtcggttgcaggggtgtcggtgtggccgcagacgattgattatcaggaggatgtgaggtgtagttctgcgaaggcgttttattatcctgttgaggggagggagaatctgagggttgtgaaggggaccgtcagaaggattctttgggctgacacccggggaggagagcatgtggcggcgggagtcgagtatctggatgagaacggacagatgcgaactgcaacggcccgcaaagaagtgatcctgtcagctggggcactgcggacaccccccatcctggaagcatcaggcgttggcgacgcagacaggctcagaggacttgggatagagacgcgaatcgatctccctggcgtaggagagaacctgcaagaccaggcaaacgtgcctctgctatacacgggaaacctcaatatctcagggacctcgccgtacgccacgttcctcatggcatcacagctctttggcgagaatctcgaagctgtagctgcagagacactctcctccgttccgtcctgggccgactccctcgcctcatcctcatccaacaacctcaacagcagcgcgatagaacgcctcctcaccctccagcacgccctcatcttcaaatccaacgtcactatagcagaaattctcaccagcgcgtccggaaccaccctcctctctgccttctggctcctcctccctttctcccggggcagcgtgcacctctcctcaacaaggcgagaagatataaacgcacccagcattgacacaaatttcttccaggtggactttgatctccagaccgagatggccatagggagactagcgcagtcattctgggaacaggggcccgtgaagagtctacatcccgttccgatgcccggcagggcattggatgataatgcaacagacacagaatggacagcgtttactaaagaaacctgtgagttgcatctggcagagaaaaatgaagaaaaggggcatactgattga

SEQ ID NO: 15 - protein, 544 aa, Aspergillus
MKLQRLLSITLTFTITAPTATATASASAKADAEAEADYLVTGGGTTGLLLANRLSSTPTTTVLILDPGNDIRTNPNVTDPTLWLRNAHTEVDWAYPSTPQSHALNRILSYTAGRILGGTSMINGMTYLRADKPEIDAWEALGAKGWNWGSLWPYYLRTEKFSPPLGWQVGAGADYVPDLHGRTGSVDVCFSMELSRVGFWERVRDAWRVLGVNWNRDPNGGSVAGVSVWPQTIDYQEDVRCSSAKAFYYPVEGRENLRVVKGTVRRILWADTRGGEHVAAGVEYLDENGQMRTATARKEVILSAGALRTPPILEASGVGDADRLRGLGIETRIDLPGVGENLQDQANVPLLYTGNLNISGTSPYATFLMASQLFGENLEAVAAETLSSVPSWADSLASSSSNNLNSSAIERLLTLQHALIFKSNVTIAEILTSASGTTLLSAFWLLLPFSRGSVHLSSTRREDINAPSIDTNFFQVDFDLQTEMAIGRLAQSFWEQGPVKSLHPVPMPGRALDDNATDTEWTAFTKETCELHLAEKNEEKGHTD

SEQ ID NO: 16 - DNA, 1943 bp, Aspergillus
atgattctcagtgcgttcattctccttctcactctggtcactgctctggcccccgaatatgacttcatcatcgtcggaggaggagtcagcggccttgtcgtcgccaaccgtctctccgaggaccccaacgtctccgttctcatcatcgaagccggtccatcagtccttgacaacgagaatgtgaccgatgtcgacgcgtactcccgtgcgtttggtaccgaaatagactggcagttcatcagtgaatcacagctcttcggcggagaaccacagatcctccgggccggacgagcgctgggtggcgggagcgcaatcaatggtgaatcatccccatccggatatggatatgaatatgcagaagagctaagactgcgtttaggaatggcctacgtgcgtgctgaggacgtccaactggacgcttggcagtccatcggcaatgagcgctggaactggacgagtctgtttccttactatctcaagagcgagaacctgaccttgccgacagccgtgcagacggatgcaggcgcaacatacgatgcctttgcgcatggcacccgcggtcccctcaaggttgctttcccacgtatgcagagtggtgataatgatctgacacctgcggtcaaccagacgcttcaggctgcggggattccgtggaacgtagatgtgaatgctgggcgcatgcgtggatttagtatttacccctggaccatcgatgaggaggcgtatattcggtatgatgccgctcgggcttatttttggccgtttcagtcgcgatcgaacctgcatgcctggttgaacacgagggtgaatcggattgtttggagagacgtgcctggtggagagaacacccgtgcagctggcgtggaggttacttcacagcatggcactgtaagtgttgtgatgtcgagaagagaggtgattctctcggctggagcgctcaagtcgcccgccattcttgagctttccgggatcgggaatccacggtgagttgcttctcggtcttatatccgcgataaacgggcttgagctgaggcactgaaactcccttatagcatcctcaatcaacacaacatccccgttcacgtctcactcccaggcgtcggagaaaacttacaggatcagatgaacactctcatgacagcgtcgacccattcacccatcaccggcggcagaaccgtcactttcgcatcggccacggacatcttcagtcaggatatctcctccctcgccaacctcacacataccaagctcccctcgtacgctgccctcgtcgcaaacatgagcaacggagccatgcaacccgaacacctgcgggctgtattccaagtgcaacacgaccttatcttcaagaacaacattcccattgcagaaataatcttcaagcccggcggcgagaaaaccgtcaacgccgggttctggggcctcctcccctttgcacgcgggaacgtgcacatccgctcttctgatccggcagcccagcccgcgatcagtcccaattacggcctgttggactgggatatccagttgcaggttgcaattgccaagttcatccggcggatgtttcgctctgcgccgcttgaggggatgattgaggaggaatcgaggcctggtctgtcggcggtgcctggggatgccgcggatgaggtgtgggggaactggttggaagataactgtacgtggggtaattttctctttttcctaggagatgtctctaactcagtgctgtagatgcgtccaacttccatgccatcggcacagcggccatgatgccccgttctctaggtggagtcgtcaacgatcggctgcaggtgtacggcacggcgaatgttcgcgttgtcgatgcgtctattcatccgcttcagctttgcggtcatccgatggctaacctgtacgctattgcagagcgcacagcggatttgatcaaggaggattggacgggctaa

SEQ ID NO: 17 - DNA, 1758 bp, Aspergillus
  CDS (1)...(1758)
atgattctcagtgcgttcattctccttctcactctggtcactgctctggcccccgaatatgacttcatcatcgtcggaggaggagtcagcggccttgtcgtcgccaaccgtctctccgaggaccccaacgtctccgttctcatcatcgaagccggtccatcagtccttgacaacgagaatgtgaccgatgtcgacgcgtactcccgtgcgtttggtaccgaaatagactggcagttcatcagtgaatcacagctcttcggcggagaaccacagatcctccgggccggacgagcgctgggtggcgggagcgcaatcaatggaatggcctacgtgcgtgctgaggacgtccaactggacgcttggcagtccatcggcaatgagcgctggaactggacgagtctgtttccttactatctcaagagcgagaacctgaccttgccgacagccgtgcagacggatgcaggcgcaacatacgatgcctttgcgcatggcacccgcggtcccctcaaggttgctttcccacgtatgcagagtggtgataatgatctgacacctgcggtcaaccagacgcttcaggctgcggggattccgtggaacgtagatgtgaatgctgggcgcatgcgtggatttagtatttacccctggaccatcgatgaggaggcgtatattcggtatgatgccgctcgggcttatttttggccgtttcagtcgcgatcgaacctgcatgcctggttgaacacgagggtgaatcggattgtttggagagacgtgcctggtggagagaacacccgtgcagctggcgtggaggttacttcacagcatggcactgtaagtgttgtgatgtcgagaagagaggtgattctctcggctggagcgctcaagtcgcccgccattcttgagctttccgggatcgggaatccacgcatcctcaatcaacacaacatccccgttcacgtctcactcccaggcgtcggagaaaacttacaggatcagatgaacactctcatgacagcgtcgacccattcacccatcaccggcggcagaaccgtcactttcgcatcggccacggacatcttcagtcaggatatctcctccctcgccaacctcacacataccaagctcccctcgtacgctgccctcgtcgcaaacatgagcaacggagccatgcaacccgaacacctgcgggctgtattccaagtgcaacacgaccttatcttcaagaacaacattcccattgcagaaataatcttcaagcccggcggcgagaaaaccgtcaacgccgggttctggggcctcctcccctttgcacgcgggaacgtgcacatccgctcttctgatccggcagcccagcccgcgatcagtcccaattacggcctgttggactgggatatccagttgcaggttgcaattgccaagttcatccggcggatgtttcgctctgcgccgcttgaggggatgattgaggaggaatcgaggcctggtctgtcggcggtgcctggggatgccgcggatgaggtgtgggggaactggttggaagataactatgcgtccaacttccatgccatcggcacagcggccatgatgccccgttctctaggtggagtcgtcaacgatcggctgcaggtgtacggcacggcgaatgttcgcgttgtcgatgcgtctattcatccgcttcagctttgcggtcatccgatggctaacctgtacgctattgcagagcgcacagcggatttgatcaaggaggattggacgggctaa

SEQ ID NO: 18 - protein, 585 aa, Aspergillus
MILSAFILLLTLVTALAPEYDFIIVGGGVSGLVVANRLSEDPNVSVLIIEAGPSVLDNENVTDVDAYSRAFGTEIDWQFISESQLFGGEPQILRAGRALGGGSAINGMAYVRAEDVQLDAWQSIGNERWNWTSLFPYYLKSENLTLPTAVQTDAGATYDAFAHGTRGPLKVAFPRMQSGDNDLTPAVNQTLQAAGIPWNVDVNAGRMRGFSIYPWTIDEEAYIRYDAARAYFWPFQSRSNLHAWLNTRVNRIVWRDVPGGENTRAAGVEVTSQHGTVSVVMSRREVILSAGALKSPAILELSGIGNPRILNQHNIPVHVSLPGVGENLQDQMNTLMTASTHSPITGGRTVTFASATDIFSQDISSLANLTHTKLPSYAALVANMSNGAMQPEHLRAVFQVQHDLIFKNNIPIAEIIFKPGGEKTVNAGFWGLLPFARGNVHIRSSDPAAQPAISPNYGLLDWDIQLQVAIAKFIRRMFRSAPLEGMIEEESRPGLSAVPGDAADEVWGNWLEDNYASNFHAIGTAAMMPRSLGGVVNDRLQVYGTANVRVVDASIHPLQLCGHPMANLYAIAERTADLIKEDWTG

SEQ ID NO: 19 - DNA, 2353 bp, Aspergillus
atgcagcaagaaggggtctcaggtggttcgtctctttcggtcttggagtgtgaccgctcagcttctcgtttcaattttccttatttccctaccatcatcatcatcattatcatcatcatcatcactgtgatctattcaaggcagtcatcgataagagcatgtgaaagttgtgcatgcgaggcagtctccatccgccttgatgaaatgaaagatgaaacgccgcgaatgaccgagatgacaaggaagccgttcctgacgatcgccataggcgtcttgaccactgtcagcgctctctcggaccccgctcagattgtgattccagcaacaataacaccccagacgacggaccccttggtgtcatggttagcgcaagaaacttcatacgccctcgatggtgtgttaaacaatgtcggaccgaacggggctaaggcaactggcgctagctctggcatcatcatcgctagtcctagccaaagcaatccggactgtgagtaattacttcctccgtaccatttcactgcaactgactcggctgacctgtggttgagactactatacctggactcgagacgccgctctcaccgtcaaatacctcgttggttcttttgccgccgatcacgatcctgcaatccaaaggatcatagaggattacgtagaatcccaggcgcacttgcagactgtctccaacccttccggaaatctatcaagcggaggcttgggggaacctaagttgagggtggatggatcggcattccatggctcctgggggcgtccacagagtgatgggcctgcgctgagagcaacaacactgatatcctacgcaaacttgatgattgtatgtttttgctcttttcaataaagattgagcttcctaacttcgtgtactctaataggataatggttatttctctaccgttgagtcaagcatctggccaatcattcagaatgacctttcctatctaacggagttttggaactcttctacttttggtacgtttgtgtgtttctcatacgggaggatcatccagtgaccagatccggtagatctttgggaggaagttcgcggttcctcatttttcaccacggcagtccagcatcaagccctcagaaaaggtgcagcccttgcccagaggcttggtaaaacctgttcaaactgccagtcacaggcaccgcaggttctctgtttcctgcagacttactggactggctcgtccatactggccaatctgtatagtgatcggtccgggaaagatgccaattcaatactaggcatcatccatacttttgaccccaatgcgggctgcgacggacagacgttccaaccgtgttctgatcgggcgcttgcaagccacaaggaagtcgtagactccttccgctcgctgtacccgatcaatgcagacattccccagggccaagctgtagccgtgggtcggtacccagaggatgtctatcaaggtggacacccttggtatctgtgcactttggcagcagctgagcagctatacgatgcactttatcagtgggagagattggggtccctcgccgttacaaatataagcctcgcgttcttccaggacttggtcccatcagctacagttgggacatacgcgaaggacaccatcacatttgcctctatatctgcagctgttcgagattacgcagatcgatttctccgaatcgtggtaggtgttactctctaactttttttttggccagaaccgtgtcaagcactaacatgacctcacagcagaaatacacacccccgaatggcgccctcgcagagcaattctcgcgctatgacggctcccccctctcagcgcaagacctcacttggtcctacacatcctttctcactgcagttcaagcccgaagacatgctcttaatccttccgcatcacacatccaacctctcctctcaaacgcgaccaccgccactgccctcccccaagtctgcaccccttcctccgcacgagggccctaccagcccgttaaacgaatcaagtggcccagaccagagtgtctctcccctcggagtaccgtcgccgttcggttcaatgtcctggcgaccaccgttattggcgaggacatcttcctcgtcgggtccatccccgcgctgggcgaatgggatgcgcggcatcaggctttgaagttggaagcgaatgagtactcgagcatcacgcccttgtggtacaggaccgtgatgttggatgccggggaggagtttgaatataaattcattcggaaaagggtgggtgacggtaaggtaatctgggaagaaggccagaatcgagcatttgtggtgccgagagagtgtggcccttccaatgcgacaatcaatgccgtctggagatga

SEQ ID NO: 20 - DNA, 2115 bp, Aspergillus
  CDS (1)...(2115)
atgcagcaagaaggggtctcaggtggttcgtctctttcggtcttggagtgtgaccgctcagcttctcgtttcaattttccttatttccctaccatcatcatcatcattatcatcatcatcatcactgtgatctattcaaggcagtcatcgataagagcatgtgaaagttgtgcatgcgaggcagtctccatccgccttgatgaaatgaaagatgaaacgccgcgaatgaccgagatgacaaggaagccgttcctgacgatcgccataggcgtcttgaccactgtcagcgctctctcggaccccgctcagattgtgattccagcaacaataacaccccagacgacggaccccttggtgtcatggttagcgcaagaaacttcatacgccctcgatggtgtgttaaacaatgtcggaccgaacggggctaaggcaactggcgctagctctggcatcatcatcgctagtcctagccaaagcaatccggactactactatacctggactcgagacgccgctctcaccgtcaaatacctcgttggttcttttgccgccgatcacgatcctgcaatccaaaggatcatagaggattacgtagaatcccaggcgcacttgcagactgtctccaacccttccggaaatctatcaagcggaggcttgggggaacctaagttgagggtggatggatcggcattccatggctcctgggggcgtccacagagtgatgggcctgcgctgagagcaacaacactgatatcctacgcaaacttgatgattgataatggttatttctctaccgttgagtcaagcatctggccaatcattcagaatgacctttcctatctaacggagttttggaactcttctacttttgatctttgggaggaagttcgcggttcctcatttttcaccacggcagtccagcatcaagccctcagaaaaggtgcagcccttgcccagaggcttggtaaaacctgttcaaactgccagtcacaggcaccgcaggttctctgtttcctgcagacttactggactggctcgtccatactggccaatctgtatagtgatcggtccgggaaagatgccaattcaatactaggcatcatccatacttttgaccccaatgcgggctgcgacggacagacgttccaaccgtgttctgatcgggcgcttgcaagccacaaggaagtcgtagactccttccgctcgctgtacccgatcaatgcagacattccccagggccaagctgtagccgtgggtcggtacccagaggatgtctatcaaggtggacacccttggtatctgtgcactttggcagcagctgagcagctatacgatgcactttatcagtgggagagattggggtccctcgccgttacaaatataagcctcgcgttcttccaggacttggtcccatcagctacagttgggacatacgcgaaggacaccatcacatttgcctctatatctgcagctgttcgagattacgcagatcgatttctccgaatcgtgcagaaatacacacccccgaatggcgccctcgcagagcaattctcgcgctatgacggctcccccctctcagcgcaagacctcacttggtcctacacatcctttctcactgcagttcaagcccgaagacatgctcttaatccttccgcatcacacatccaacctctcctctcaaacgcgaccaccgccactgccctcccccaagtctgcaccccttcctccgcacgagggccctaccagcccgttaaacgaatcaagtggcccagaccagagtgtctctcccctcggagtaccgtcgccgttcggttcaatgtcctggcgaccaccgttattggcgaggacatcttcctcgtcgggtccatccccgcgctgggcgaatgggatgcgcggcatcaggctttgaagttggaagcgaatgagtactcgagcatcacgcccttgtggtacaggaccgtgatgttggatgccggggaggagtttgaatataaattcattcggaaaagggtgggtgacggtaaggtaatctgggaagaaggccagaatcgagcatttgtggtgccgagagagtgtggcccttccaatgcgacaatcaatgccgtctggagatga

SEQ ID NO: 21 - protein, 704 aa, Aspergillus
MQQEGVSGGSSLSVLECDRSASRFNFPYFPTIIIIIIIIIITVIYSRQSSIRACESCACEAVSIRLDEMKDETPRMTEMTRKPFLTIAIGVLTTVSALSDPAQIVIPATITPQTTDPLVSWLAQETSYALDGVLNNVGPNGAKATGASSGIIIASPSQSNPDYYYTWTRDAALTVKYLVGSFAADHDPAIQRIIEDYVESQAHLQTVSNPSGNLSSGGLGEPKLRVDGSAFHGSWGRPQSDGPALRATTLISYANLMIDNGYFSTVESSIWPIIQNDLSYLTEFWNSSTFDLWEEVRGSSFFTTAVQHQALRKGAALAQRLGKTCSNCQSQAPQVLCFLQTYWTGSSILANLYSDRSGKDANSILGIIHTFDPNAGCDGQTFQPCSDRALASHKEVVDSFRSLYPINADIPQGQAVAVGRYPEDVYQGGHPWYLCTLAAAEQLYDALYQWERLGSLAVTNISLAFFQDLVPSATVGTYAKDTITFASISAAVRDYADRFLRIVQKYTPPNGALAEQFSRYDGSPLSAQDLTWSYTSFLTAVQARRHALNPSASHIQPLLSNATTATALPQVCTPSSARGPYQPVKRIKWPRPECLSPRSTVAVRFNVLATTVIGEDIFLVGSIPALGEWDARHQALKLEANEYSSITPLWYRTVMLDAGEEFEYKFIRKRVGDGKVIWEEGQNRAFVVPRECGPSNATINAVWR

SEQ ID NO: 22 - DNA, 1455 bp, Aspergillus
atggtgactctgactttcctgctttcggcggcgtatctgctttctgggtgagtggcttggatctattgctcggatagggctgtggtgctgattctgaaacggagtagagtgtctgcggcacctagttctgctggctccaagtcctgcgatacggtagacctcgggtaccagtgctcccctgcgacttctcatctatggggccagtactcgccattcttttcgctcgaggacgagctgtccgtgtcgagtaagcttcccaaggattgccggatcaccttggtacaggtgctatcgcgccatggagcgcggtacccaaccagctccaagagcaaaaagtataagaagcttgtgacggcgatccaggccaatgccaccgacttcaagggcaagtttgcctttttgaagacgtacaactatactctgggtgcggatgacctcactccctttggggagcagcagctggtgaactcgggcatcaagttctaccagaggtacaaggctctggcgcgcagtgtggtgccgtttattcgcgcctcaggctcggaccgggttattgcctcgggagagaagttcatcgaggggttccagcaggcgaagctggctgatcctggcgcgacgaaccgcgccgctccggcgattagtgtgattattccggagagcgagacgttcaacaatacgctggaccacggtgtgtgcacgaagtttgaggcgagtcagctgggagatgaggttgcggccaatttcactgcgctctttgcacccgacatccgagctcgcgccgagaagcatcttcctggcgtgacgctgacagacgaggacgttgtcagtctaatggacatgtgttcgtttgatacggtagcgcgcaccagcgacgcaagtcagctgtcaccgttctgtcaactcttcactcacaatgagtggaagaagtacaactaccttcagtccttgggcaagtactacggctacggcgcaggcaaccctctgggaccggctcaggggatagggttcaccaacgagctgattgcccggttgactcgttcgccagtgcaggaccacaccagcactaactcgactctagtctccaacccggccaccttcccgttgaacgctaccatgtacgtcgacttttcacacgacaacagcatggtttccatcttctttgcattgggcctgtacaacggcactgaacccttgtcccggacctcggtggaaagcgccaaggaattggatgggtattctgcatcctgggtggtgcctttcggcgcgcgagcctacttcgagacgatgcaatgcaagtcggaaaaggagcctcttgttcgcgctttgattaatgaccgggttgtgccactgcatggctgcgatgtggacaagctggggcgatgcaagctgaatgactttgtcaagggattgagttgggccagatctgggggcaactggggagagtgctttagttga

SEQ ID NO: 23 - DNA, 1425 bp, Aspergillus
  CDS (1)...(1425)
atggtgactctgactttcctgctttcggcggcgtatctgctttctggggctgtggtgctgattctgaaacggagtagagtgtctgcggcacctagttctgctggctccaagtcctgcgatacggtagacctcgggtaccagtgctcccctgcgacttctcatctatggggccagtactcgccattcttttcgctcgaggacgagctgtccgtgtcgagtaagcttcccaaggattgccggatcaccttggtacaggtgctatcgcgccatggagcgcggtacccaaccagctccaagagcaaaaagtataagaagcttgtgacggcgatccaggccaatgccaccgacttcaagggcaagtttgcctttttgaagacgtacaactatactctgggtgcggatgacctcactccctttggggagcagcagctggtgaactcgggcatcaagttctaccagaggtacaaggctctggcgcgcagtgtggtgccgtttattcgcgcctcaggctcggaccgggttattgcctcgggagagaagttcatcgaggggttccagcaggcgaagctggctgatcctggcgcgacgaaccgcgccgctccggcgattagtgtgattattccggagagcgagacgttcaacaatacgctggaccacggtgtgtgcacgaagtttgaggcgagtcagctgggagatgaggttgcggccaatttcactgcgctctttgcacccgacatccgagctcgcgccgagaagcatcttcctggcgtgacgctgacagacgaggacgttgtcagtctaatggacatgtgttcgtttgatacggtagcgcgcaccagcgacgcaagtcagctgtcaccgttctgtcaactcttcactcacaatgagtggaagaagtacaactaccttcagtccttgggcaagtactacggctacggcgcaggcaaccctctgggaccggctcaggggatagggttcaccaacgagctgattgcccggttgactcgttcgccagtgcaggaccacaccagcactaactcgactctagtctccaacccggccaccttcccgttgaacgctaccatgtacgtcgacttttcacacgacaacagcatggtttccatcttctttgcattgggcctgtacaacggcactgaacccttgtcccggacctcggtggaaagcgccaaggaattggatgggtattctgcatcctgggtggtgcctttcggcgcgcgagcctacttcgagacgatgcaatgcaagtcggaaaaggagcctcttgttcgcgctttgattaatgaccgggttgtgccactgcatggctgcgatgtggacaagctggggcgatgcaagctgaatgactttgtcaagggattgagttgggccagatctgggggcaactggggagagtgctttagttga

SEQ ID NO: 24 - protein, 474 aa, Aspergillus
MVTLTFLLSAAYLLSGAVVLILKRSRVSAAPSSAGSKSCDTVDLGYQCSPATSHLWGQYSPFFSLEDELSVSSKLPKDCRITLVQVLSRHGARYPTSSKSKKYKKLVTAIQANATDFKGKFAFLKTYNYTLGADDLTPFGEQQLVNSGIKFYQRYKALARSVVPFIRASGSDRVIASGEKFIEGFQQAKLADPGATNRAAPAISVIIPESETFNNTLDHGVCTKFEASQLGDEVAANFTALFAPDIRARAEKHLPGVTLTDEDVVSLMDMCSFDTVARTSDASQLSPFCQLFTHNEWKKYNYLQSLGKYYGYGAGNPLGPAQGIGFTNELIARLTRSPVQDHTSTNSTLVSNPATFPLNATMYVDFSHDNSMVSIFFALGLYNGTEPLSRTSVESAKELDGYSASWVVPFGARAYFETMQCKSEKEPLVRALINDRVVPLHGCDVDKLGRCKLNDFVKGLSWARSGGNWGECFS

SEQ ID NO: 25 - DNA, 2997 bp, Aspergillus
atgaagtcccttttaaagagattgattgcactggccgccgcttattcggtagcagctgcaccatcctttagccaccattcatctcaagatgctgcgaataagagagagcttcttcaagacttagtgacgtgggaccaacactccctgtttgtccggggcgagcggctcatgatcttctctggagagtttcacccttttcgactgccagttcctggactctggtttgatgtcttccagaagattaagagtctgggattcaatgctgtctccttctatactgactggggtctcatggagggtaatcctggccacgttgtgactgacggtatctggagccttgacgagtttttcactgctgcacgtgaagccggtctctacctgatcgctcggccagggccatatatcaatgcggaaacctcggctggtggtatacccggatgggttctccgtcgaaagggcatcatccgctcaaacagtgaggattacctccgtgcgacagatacgtacatggccactctagggaagataattgccaaggcgcaaattaccaatggagggccggtgatcttggttcagccagaaaatgagtacacgacttggcccaacgtctcagagtccgagtttccgacgaccatgaatcaggaagtcatggcctatgccgagaagcagcttcgagacgctggagtagttgttccaaccgttgtaaatgacaataagaacctgggatactttgcccctgggactggactgggcgagacggatctctacgggattgacgcctatcctatgagatatgactgtgagaccattactttcaagagatgattggatctgatgcagaatcaggtggtaacccctatgtatggccaacatatcgattcccgcgagattggcagcatgaacaccgcaaccatagccctacaacgccatttgctattatggagttccaagggggttcaggggatgggtggtaagagatacataccatgggtactgcccctatgaacgcaatgactaacataatcaccaggggaggagtcacagaagacggatgtgccattcttgtcaacaatgaagctgtgcgagtggtgtacaagaacaattacggatttggcgtcagagtgttcaacatctacatggtgtgaaatgctccctcctcctcctccctacatccgctcctaactgactgtgtagacgtacggaggcaccaattggggcaatctcggctactatggaggatacacctcgtatgactacggggctgccattaccgaggatcgtcagatctggcgagaaaagtatagcgaagagaagctacaggcaaactttctgaaagtgtctccggcgtacctcacctcaacgcctggaaacggggtcaatggatcatacacaggaaacaaggatataacggtcactccgttgtttggcaatggaacgaccaccaatctctatctagtccggcatgccgactttacttcgactggaagtgcccaatacaatctcagcatttccacgagcgtcggaaatgtgacgatccctcaactaggaggcagtctctccttgaacggccgagactccaaattccacatcacggactatgatgtgggtggattcaatctcatctactcttctgcagaagtcttcacttgggcgaagggagacaacaagaagcgcgttctcgtgctatatggaggagcaggggaactacatgagttcgcgctgcccaaacatcttccgcggccaaccgttgttgaggggtcctacgtcaagattgccaaacagggaagtgcctgggtcgtacagtgggaggtagctgctcaaagacgggttttacgtgcaggaaagcttgagatccacctcctctggcgtaacgacgcctaccagcactgggtcttggaactaccagcaaaacaacccattgctaactatagctcgccatccaaagagacggtgattgtgaagggaggatatttgcttcgaagcgcgtggattactgacaatgacctgcatttgactggtgatgtgaacgtaacaactcccttggaggtgatttcagcgccgaagcgatttgatgggattgtcttcaacggccagtccttgaaatcaacacgatctaagattggcaatctcgctgcaacagttcactatcaaccaccagctatctctctgccagatctaaagcgtcttgactggaaatacatcgattctctcccagagatctccacggaatataacgacgagggctggacaccactgacgaacacatacaccaataacaccagagagttcactggccctacatgtctgtacgccgatgactacggctatcacgggggctcactgatctaccggggccacttcacggccaacggagacgagtcatgggttttcctcaacacctctggcggtgtaggcttcgccaattcggtatggctaaaccaaacattcctgggttcatggacaggcagtggaagaaacatgacctatccccgaaacatctcgcttccccacgagctgtctcctggggaaccgtacgttttcaccgtggtgatcgaccacatgggccaggacgaagaggcaccggggacagacgcaatcaagtttcctcgaggaattctcgactacgcgctctcgggccatgagctgtccgatctccgatggaagatgactggcaacctcggcggcgagcagtaccaggatttgacgcgaggtccgctcaacgagggagccatgtatgccgagcggcagggatatcatcttccaagtccgcctacttccagctggaagtcatccaacccgattaaggaagggctcactggcgctggtattggattctatgctacttcgttctccttggatctacccgagggatacgatatcccattgagctttcggttcaacaactcggcatcagccgcgaggtcagggacgagctatcgctgtcagctgtttgttaatggataccagtttggaaaatacggtatgataagaagttga

SEQ ID NO: 26 - DNA, 2835 bp, Aspergillus
  CDS (1)...(2835)
atgaagtcccttttaaagagattgattgcactggccgccgcttattcggtagcagctgcaccatcctttagccaccattcatctcaagatgctgcgaataagagagagcttcttcaagacttagtgacgtgggaccaacactccctgtttgtccggggcgagcggctcatgatcttctctggagagtttcacccttttcgactgccagttcctggactctggtttgatgtcttccagaagattaagagtctgggattcaatgctgtctccttctatactgactggggtctcatggagggtaatcctggccacgttgtgactgacggtatctggagccttgacgagtttttcactgctgcacgtgaagccggtctctacctgatcgctcggccagggccatatatcaatgcggaaacctcggctggtggtatacccggatgggttctccgtcgaaagggcatcatccgctcaaacagtgaggattacctccgtgcgacagatacgtacatggccactctagggaagataattgccaaggcgcaaattaccaatggagggccggtgatcttggttcagccagaaaatgagtacacgacttggcccaacgtctcagagtccgagtttccgacgaccatgaatcaggaagtcatggcctatgccgagaagcagcttcgagacgctggagtagttgttccaaccgttgtaaatgacaataagaacctgggatactttgcccctgggactggactgggcgagacggatctctacgggattgacgcctatcctatgagatatgactgtggtaacccctatgtatggccaacatatcgattcccgcgagattggcagcatgaacaccgcaaccatagccctacaacgccatttgctattatggagttccaagggggttcaggggatgggtggggaggagtcacagaagacggatgtgccattcttgtcaacaatgaagctgtgcgagtggtgtacaagaacaattacggatttggcgtcagagtgttcaacatctacatgacgtacggaggcaccaattggggcaatctcggctactatggaggatacacctcgtatgactacggggctgccattaccgaggatcgtcagatctggcgagaaaagtatagcgaagagaagctacaggcaaactttctgaaagtgtctccggcgtacctcacctcaacgcctggaaacggggtcaatggatcatacacaggaaacaaggatataacggtcactccgttgtttggcaatggaacgaccaccaatctctatctagtccggcatgccgactttacttcgactggaagtgcccaatacaatctcagcatttccacgagcgtcggaaatgtgacgatccctcaactaggaggcagtctctccttgaacggccgagactccaaattccacatcacggactatgatgtgggtggattcaatctcatctactcttctgcagaagtcttcacttgggcgaagggagacaacaagaagcgcgttctcgtgctatatggaggagcaggggaactacatgagttcgcgctgcccaaacatcttccgcggccaaccgttgttgaggggtcctacgtcaagattgccaaacagggaagtgcctgggtcgtacagtgggaggtagctgctcaaagacgggttttacgtgcaggaaagcttgagatccacctcctctggcgtaacgacgcctaccagcactgggtcttggaactaccagcaaaacaacccattgctaactatagctcgccatccaaagagacggtgattgtgaagggaggatatttgcttcgaagcgcgtggattactgacaatgacctgcatttgactggtgatgtgaacgtaacaactcccttggaggtgatttcagcgccgaagcgatttgatgggattgtcttcaacggccagtccttgaaatcaacacgatctaagattggcaatctcgctgcaacagttcactatcaaccaccagctatctctctgccagatctaaagcgtcttgactggaaatacatcgattctctcccagagatctccacggaatataacgacgagggctggacaccactgacgaacacatacaccaataacaccagagagttcactggccctacatgtctgtacgccgatgactacggctatcacgggggctcactgatctaccggggccacttcacggccaacggagacgagtcatgggttttcctcaacacctctggcggtgtaggcttcgccaattcggtatggctaaaccaaacattcctgggttcatggacaggcagtggaagaaacatgacctatccccgaaacatctcgcttccccacgagctgtctcctggggaaccgtacgttttcaccgtggtgatcgaccacatgggccaggacgaagaggcaccggggacagacgcaatcaagtttcctcgaggaattctcgactacgcgctctcgggccatgagctgtccgatctccgatggaagatgactggcaacctcggcggcgagcagtaccaggatttgacgcgaggtccgctcaacgagggagccatgtatgccgagcggcagggatatcatcttccaagtccgcctacttccagctggaagtcatccaacccgattaaggaagggctcactggcgctggtattggattctatgctacttcgttctccttggatctacccgagggatacgatatcccattgagctttcggttcaacaactcggcatcagccgcgaggtcagggacgagctatcgctgtcagctgtttgttaatggataccagtttggaaaatacggtatgataagaagttga

SEQ ID NO: 27 - protein, 944 aa, Aspergillus
MKSLLKRLIALAAAYSVAAAPSFSHHSSQDAANKRELLQDLVTWDQHSLFVRGERLMIFSGEFHPFRLPVPGLWFDVFQKIKSLGFNAVSFYTDWGLMEGNPGHVVTDGIWSLDEFFTAAREAGLYLIARPGPYINAETSAGGIPGWVLRRKGIIRSNSEDYLRATDTYMATLGKIIAKAQITNGGPVILVQPENEYTTWPNVSESEFPTTMNQEVMAYAEKQLRDAGVVVPTVVNDNKNLGYFAPGTGLGETDLYGIDAYPMRYDCGNPYVWPTYRFPRDWQHEHRNHSPTTPFAIMEFQGGSGDGWGGVTEDGCAILVNNEAVRVVYKNNYGFGVRVFNIYMTYGGTNWGNLGYYGGYTSYDYGAAITEDRQIWREKYSEEKLQANFLKVSPAYLTSTPGNGVNGSYTGNKDITVTPLFGNGTTTNLYLVRHADFTSTGSAQYNLSISTSVGNVTIPQLGGSLSLNGRDSKFHITDYDVGGFNLIYSSAEVFTWAKGDNKKRVLVLYGGAGELHEFALPKHLPRPTVVEGSYVKIAKQGSAWVVQWEVAAQRRVLRAGKLEIHLLWRNDAYQHWVLELPAKQPIANYSSPSKETVIVKGGYLLRSAWITDNDLHLTGDVNVTTPLEVISAPKRFDGIVFNGQSLKSTRSKIGNLAATVHYQPPAISLPDLKRLDWKYIDSLPEISTEYNDEGWTPLTNTYTNNTREFTGPTCLYADDYGYHGGSLIYRGHFTANGDESWVFLNTSGGVGFANSVWLNQTFLGSWTGSGRNMTYPRNISLPHELSPGEPYVFTVVIDHMGQDEEAPGTDAIKFPRGILDYALSGHELSDLRWKMTGNLGGEQYQDLTRGPLNEGAMYAERQGYHLPSPPTSSWKSSNPIKEGLTGAGIGFYATSFSLDLPEGYDIPLSFRFNNSASAARSGTSYRCQLFVNGYQFGKYGMIRS

SEQ ID NO: 28 - DNA, 3433 bp, Aspergillus
atgaagcttctctctgtctgcgcaattgccttgttggcagctcaggcagcgggtgcttccatcaagcacatgctcaatggcttcactcttatggagcactcggatcctgcgaagcgggaactgctacagaaatacgtatgtgggatcgaaaacttggtaatcgattaatgtgtactcagacatagctctaggtcacctgggatgagaagtcactgtttgtcaatggcgagagaatcatgattttcagcggcgaagttcaccctttcaggtgcgacacagacgcggtgatctactctggtttgttcttacattcttacattgtgtagactgcccgttccttcgctgtggctcgatgtctttcagaaaatcaaggccctggggttcaattgcgtttcattctacgtcgactgggccctcctcgaaggaaaacctggcgagtacagggctgaaggcaactttgccctcgagcctttctttgacgtggctaagcaggcaggaatctatctcctcgctcgtcctggaccttatatcaatgctgaggcttcgggcggtggtttccctggatggttgcagagagtgaacggaaccctgcggacatccgatcccgcgtacctgaaggctactgataagtatgtcgcgtacgattgtttgtagcctcgaagctgctaactctgaatcagctacattgcccatgttgctgctactattgccaaaggccaaatcacaaacgggggccccgtcattctttatcaaccggagaatgagtacagcggggcgtgttgtgatgctactttccccgatggggactacatgcaatatgtcatcgaccaggctcgaaacgccggtattgttgtaccattgatcaataacgatgcctggactggaggacacaatgcccctgggacgggcaagggcgaggttgacatctatggtcacgatagctatccgtgagtcaaattccaccttctgggaagacaatcctaacttgctttgattagactaggatttgactgtgtaagattgagttttgttgaactctttgagcgattctgatgaaaatagggacacccaagcgtttggcccaagggcaacctgccaaccactttccggacggatcacttgaagcagagcccgacaaccccgtactctcttattgaggtgtgtgtgtgtctcaaatgccctcggatccggacaccactaatcgtcgtgcagttccaagcaggttcctttgatccatggggtggacccggctttgcagcatgcgccgccctcgtcaatcacgagttcgagagagtattctacaagaatgacctaagctttggagctgctatcctgaacttgtacatggtatggaaggatcccgcttaagattgaccaagcgctgacacgcttagacctttggcggcacgaactggggtaaccttggccatcccggtggatacacatcctacgattatggatcgccgctgactgaatctcgcaatgtcactcgagagaagtacagtgagctcaagttaatcggtaacttcgtgaaagcctcgccgtcttaccttctggctacgcctggcaatttgactacctctggatatgccgacaccgctgatttgaccgtgactcctttgctgggcaatggcactggatcgtactttgttgtcagacatacggattacaccagccaggcatctaccccgtacaaactcagcctcccaaccagtgctggaagactcacagttccccagctcggcggtactctgacgctcaatggacgtgattccaaaattcatgttgtggactacaacgtcgcggggaccaatatcatatactcgactgccgaagtcttcacctggaagaactttggggatagcaaagtcctcatcttgtatggtggacctggcgagcaccatgagctggcggtttctttcaagtccgacgtccaggtcgtcgaaggatctaattcagaattcaagtcgaagaaggtgggggatgttgctgtggttgcctgggatgtgtctccatcccgccgtattgtccagattggcgacctcaagatattcctgctgggtgagtagatccgcttgttcttcttactcaaagctgatttcgagcagataggaactctgtctacaattactgggtgccacaacttgacaaggacgactcgtcaacgggctacagctccgaaaagactactgcttcgtccattattgtcaaggctggttaccttgtgcgaaccgcttatactaagggctccggcctctatcttactgctgacttcaacgcaacaactcctgtcgaagtgatcggagccccgtcaaacgtccggaacctgtacatcaacggcgagaagacccagttcaagaccgataagaatggtatctggtcaactgaggtcaagtacagtgctcctaagatcaagcttcccagcatgaaggatctggattggaagtatcttgatactctgcaggaggttcagtcaacctatgacgattctgcgtggccagcagccgacctggacaccacacctaacaccttgcgacccttgaccacgccaaagtctctgtactcgtcggactatggcttccatacgggttacctgatctaccgcggccactttgttgctgacggcagcgagactacatttgacgtgcgcacgcaaggaggctcggcctttggcagttccgtctggctgaacgaatcattcctaggctcctggactggtctcaacgccaatgcggactacaactcaacctacaaattgccgcaggtcgagcaaggcaagaactatgtcctcactattctcattgacacaatgggcctcaacgagaactgggttgtcggcacggacgagatgaagaacccccggggtattctctcctacaagctctccggccgggacgcctccgccatcacctggaaattgaccggaaacctcggcggagaggattaccaagacaagatccgcggccctctgaacgaaggtggattgtatgccgaacggcaaggcttccaccagccccagccgcccagccagaagtggaaatcagctagtcctctcgatgggttatccaagcctggcattggcttctacaccgctcagtttgatctggatatcccaagcgggtgggacgtgccgctctacttcaactttggcaacagcacgaagtcagcctaccgggtgcagctgtatgtcaacggataccagtacggcaagttcgtcagcaacataggccctcagacaagcttccccgtcccgcagggtatcctgaactatcagggaaccaattgggttgcgttgactctctgggcactcgagtcggatggtgccaagttggatgactttgagttggtgaacaccacaccagtcatgactgccctatcaaaaattcggccatcaaagcagccaaattatcgtcaacggaagggggcgtattga

SEQ ID NO: 29 - DNA, 3075 bp, Aspergillus
  CDS (1)...(3075)
atgaagcttctctctgtctgcgcaattgccttgttggcagctcaggcagcgggtgcttccatcaagcacatgctcaatggcttcactcttatggagcactcggatcctgcgaagcgggaactgctacagaaatacgtcacctgggatgagaagtcactgtttgtcaatggcgagagaatcatgattttcagcggcgaagttcaccctttcagactgcccgttccttcgctgtggctcgatgtctttcagaaaatcaaggccctggggttcaattgcgtttcattctacgtcgactgggccctcctcgaaggaaaacctggcgagtacagggctgaaggcaactttgccctcgagcctttctttgacgtggctaagcaggcaggaatctatctcctcgctcgtcctggaccttatatcaatgctgaggcttcgggcggtggtttccctggatggttgcagagagtgaacggaaccctgcggacatccgatcccgcgtacctgaaggctactgataactacattgcccatgttgctgctactattgccaaaggccaaatcacaaacgggggccccgtcattctttatcaaccggagaatgagtacagcggggcgtgttgtgatgctactttccccgatggggactacatgcaatatgtcatcgaccaggctcgaaacgccggtattgttgtaccattgatcaataacgatgcctggactggaggacacaatgcccctgggacgggcaagggcgaggttgacatctatggtcacgatagctatccactaggatttgactgtggacacccaagcgtttggcccaagggcaacctgccaaccactttccggacggatcacttgaagcagagcccgacaaccccgtactctcttattgaggtgtgtgtgtgtctcaaatgccctcggatccggacaccactaatcgtcgtgcagttccaagcaggttcctttgatccatggggtggacccggctttgcagcatgcgccgccctcgtcaatcacgagttcgagagagtattctacaagaatgacctaagctttggagctgctatcctgaacttgtacatgacctttggcggcacgaactggggtaaccttggccatcccggtggatacacatcctacgattatggatcgccgctgactgaatctcgcaatgtcactcgagagaagtacagtgagctcaagttaatcggtaacttcgtgaaagcctcgccgtcttaccttctggctacgcctggcaatttgactacctctggatatgccgacaccgctgatttgaccgtgactcctttgctgggcaatggcactggatcgtactttgttgtcagacatacggattacaccagccaggcatctaccccgtacaaactcagcctcccaaccagtgctggaagactcacagttccccagctcggcggtactctgacgctcaatggacgtgattccaaaattcatgttgtggactacaacgtcgcggggaccaatatcatatactcgactgccgaagtcttcacctggaagaactttggggatagcaaagtcctcatcttgtatggtggacctggcgagcaccatgagctggcggtttctttcaagtccgacgtccaggtcgtcgaaggatctaattcagaattcaagtcgaagaaggtgggggatgttgctgtggttgcctgggatgtgtctccatcccgccgtattgtccagattggcgacctcaagatattcctgctggataggaactctgtctacaattactgggtgccacaacttgacaaggacgactcgtcaacgggctacagctccgaaaagactactgcttcgtccattattgtcaaggctggttaccttgtgcgaaccgcttatactaagggctccggcctctatcttactgctgacttcaacgcaacaactcctgtcgaagtgatcggagccccgtcaaacgtccggaacctgtacatcaacggcgagaagacccagttcaagaccgataagaatggtatctggtcaactgaggtcaagtacagtgctcctaagatcaagcttcccagcatgaaggatctggattggaagtatcttgatactctgcaggaggttcagtcaacctatgacgattctgcgtggccagcagccgacctggacaccacacctaacaccttgcgacccttgaccacgccaaagtctctgtactcgtcggactatggcttccatacgggttacctgatctaccgcggccactttgttgctgacggcagcgagactacatttgacgtgcgcacgcaaggaggctcggcctttggcagttccgtctggctgaacgaatcattcctaggctcctggactggtctcaacgccaatgcggactacaactcaacctacaaattgccgcaggtcgagcaaggcaagaactatgtcctcactattctcattgacacaatgggcctcaacgagaactgggttgtcggcacggacgagatgaagaacccccggggtattctctcctacaagctctccggccgggacgcctccgccatcacctggaaattgaccggaaacctcggcggagaggattaccaagacaagatccgcggccctctgaacgaaggtggattgtatgccgaacggcaaggcttccaccagccccagccgcccagccagaagtggaaatcagctagtcctctcgatgggttatccaagcctggcattggcttctacaccgctcagtttgatctggatatcccaagcgggtgggacgtgccgctctacttcaactttggcaacagcacgaagtcagcctaccgggtgcagctgtatgtcaacggataccagtacggcaagttcgtcagcaacataggccctcagacaagcttccccgtcccgcagggtatcctgaactatcagggaaccaattgggttgcgttgactctctgggcactcgagtcggatggtgccaagttggatgactttgagttggtgaacaccacaccagtcatgactgccctatcaaaaattcggccatcaaagcagccaaattatcgtcaacggaagggggcgtattga

SEQ ID NO: 30 - protein, 1024 aa, Aspergillus
MKLLSVCAIALLAAQAAGASIKHMLNGFTLMEHSDPAKRELLQKYVTWDEKSLFVNGERIMIFSGEVHPFRLPVPSLWLDVFQKIKALGFNCVSFYVDWALLEGKPGEYRAEGNFALEPFFDVAKQAGIYLLARPGPYINAEASGGGFPGWLQRVNGTLRTSDPAYLKATDNYIAHVAATIAKGQITNGGPVILYQPENEYSGACCDATFPDGDYMQYVIDQARNAGIVVPLINNDAWTGGHNAPGTGKGEVDIYGHDSYPLGFDCGHPSVWPKGNLPTTFRTDHLKQSPTTPYSLIEVCVCLKCPRIRTPLIVVQFQAGSFDPWGGPGFAACAALVNHEFERVFYKNDLSFGAAILNLYMTFGGTNWGNLGHPGGYTSYDYGSPLTESRNVTREKYSELKLIGNFVKASPSYLLATPGNLTTSGYADTADLTVTPLLGNGTGSYFVVRHTDYTSQASTPYKLSLPTSAGRLTVPQLGGTLTLNGRDSKIHVVDYNVAGTNIIYSTAEVFTWKNFGDSKVLILYGGPGEHHELAVSFKSDVQVVEGSNSEFKSKKVGDVAVVAWDVSPSRRIVQIGDLKIFLLDRNSVYNYWVPQLDKDDSSTGYSSEKTTASSIIVKAGYLVRTAYTKGSGLYLTADFNATTPVEVIGAPSNVRNLYINGEKTQFKTDKNGIWSTEVKYSAPKIKLPSMKDLDWKYLDTLQEVQSTYDDSAWPAADLDTTPNTLRPLTTPKSLYSSDYGFHTGYLIYRGHFVADGSETTFDVRTQGGSAFGSSVWLNESFLGSWTGLNANADYNSTYKLPQVEQGKNYVLTILIDTMGLNENWVVGTDEMKNPRGILSYKLSGRDASAITWKLTGNLGGEDYQDKIRGPLNEGGLYAERQGFHQPQPPSQKWKSASPLDGLSKPGIGFYTAQFDLDIPSGWDVPLYFNFGNSTKSAYRVQLYVNGYQYGKFVSNIGPQTSFPVPQGILNYQGTNWVALTLWALESDGAKLDDFELVNTTPVMTALSKIRPSKQPNYRQRKGAY

SEQ ID NO: 31 - DNA, 1977 bp, Aspergillus
atgcgatttgctggcattgctgtaggcatagccagtgtcctagggcacttggccactccttcgtctgctaaagaacttgctgcacgctcccctgcagcggcaaattcacgtctggtcaaagagggactgttcgcctacgagagcattcttgcggctcttgggaacactggcatcaatgctcctggcacagcggctggattactcattgcaagtcctaccatacagaaccctgattgtaggtgaaaaaggccccgttaggaagatcaagttgacttgatctgctccattctctagacttctatacgtggactcgggatgcggcattgactttcaaaggacttgtcgatatcttcattgggggcgatacatttatcgtcgtcaatctcgacggtcttgaaacccacatccaggactacatctcttcccaggcagttttgcagaacgtgtccaacccatccggaagactttctgacggctctggactcggcgagcccaagttcgaggtcaatttcaacccatactccggcggttggggccgtccccagcgcgatggtcctgcccttcgcgccattaccatgttgacctatattcgtcagctaatccagcaagggaaacagtctgtggcttccaacctgatctggcccgttgtagccaacgatctcacctatgtcgcgcagtactggaaccacaccggattcgatctctgggaagaaatcgatggctcttccttctttacgactgccgtgcagcaccgagcgatggttgagggtagtgcgatagctcaagcccttggtaagccacatgctgggtatgacgcggttgctcccgagattctttgcttgctgcagagctactggaacgaaagcgccattatctcgaacatcaatgtcaataacggccggtcgggcatcgatctcaactcagtcctgaccagtattcatacctttgaccctgcggctggttgtgatgattccaccttccaacaatgttcgtctaaggctttggcaaatcataaggtctatgtcgattctttccgttctatctacggtatcaacgctggccttggtcctggaaaggctgcaaatgtaggccgttacgctgaagatatttaccagggaggaaacccatggtaggtcaaaccaatctcctcacttactacgtgtcagacttctgatggattaaaggtacctcgcaactcttgcggcggcagagcagctgtatgatgccctctatcagtggaaaaagcagggttatctgaccgtcacacaaacctcgctcgccttcttccgtgacttctcgtccaccgtcgagccaggaacttacaagtcgaataccccgaactacaaatccctgactgagtatgtccgcacctacgctgatgacttcttcttccttgttgagaaatacaccccaagcaacggctccttagccgagcagtatgaccgcaacaccggtgtccctctgtcagccaacgatcttacctggtcctatgcagccttcctctctacccttcagcgccgcctcaacatcatgcctgactcctggggcccctcgtctgcaaatactgttcccactacctgcagcaaaaccaccataactggtacatacagtgctgtggcgcctccctttccgacgtccaccgctcagtgcgttgccgctgtgactgttcctgtcaccttttggttgatcgagaatacctactatggcgagaatgtgtacatgaccggcaacgtcagcgctctcggcgactggaacgccagcgcaggctatagtctgaatgccggtctctacacctcggatgagaatctttggttcgcaactgtcaagggactcgagccgggtgttaccatggagtacaagttctacaagattgaaccgggaaactccgtgacttttgaggggggggagaacagggtgtatgcagttcctacagcatgtcctgtggcgcctcaggtccacgctgtttggcagacctga

SEQ ID NO: 32 - DNA, 1863 bp, Aspergillus
  CDS (1)...(1863)
atgcgatttgctggcattgctgtaggcatagccagtgtcctagggcacttggccactccttcgtctgctaaagaacttgctgcacgctcccctgcagcggcaaattcacgtctggtcaaagagggactgttcgcctacgagagcattcttgcggctcttgggaacactggcatcaatgctcctggcacagcggctggattactcattgcaagtcctaccatacagaaccctgattacttctatacgtggactcgggatgcggcattgactttcaaaggacttgtcgatatcttcattgggggcgatacatttatcgtcgtcaatctcgacggtcttgaaacccacatccaggactacatctcttcccaggcagttttgcagaacgtgtccaacccatccggaagactttctgacggctctggactcggcgagcccaagttcgaggtcaatttcaacccatactccggcggttggggccgtccccagcgcgatggtcctgcccttcgcgccattaccatgttgacctatattcgtcagctaatccagcaagggaaacagtctgtggcttccaacctgatctggcccgttgtagccaacgatctcacctatgtcgcgcagtactggaaccacaccggattcgatctctgggaagaaatcgatggctcttccttctttacgactgccgtgcagcaccgagcgatggttgagggtagtgcgatagctcaagcccttggtaagccacatgctgggtatgacgcggttgctcccgagattctttgcttgctgcagagctactggaacgaaagcgccattatctcgaacatcaatgtcaataacggccggtcgggcatcgatctcaactcagtcctgaccagtattcatacctttgaccctgcggctggttgtgatgattccaccttccaacaatgttcgtctaaggctttggcaaatcataaggtctatgtcgattctttccgttctatctacggtatcaacgctggccttggtcctggaaaggctgcaaatgtaggccgttacgctgaagatatttaccagggaggaaacccatggtacctcgcaactcttgcggcggcagagcagctgtatgatgccctctatcagtggaaaaagcagggttatctgaccgtcacacaaacctcgctcgccttcttccgtgacttctcgtccaccgtcgagccaggaacttacaagtcgaataccccgaactacaaatccctgactgagtatgtccgcacctacgctgatgacttcttcttccttgttgagaaatacaccccaagcaacggctccttagccgagcagtatgaccgcaacaccggtgtccctctgtcagccaacgatcttacctggtcctatgcagccttcctctctacccttcagcgccgcctcaacatcatgcctgactcctggggcccctcgtctgcaaatactgttcccactacctgcagcaaaaccaccataactggtacatacagtgctgtggcgcctccctttccgacgtccaccgctcagtgcgttgccgctgtgactgttcctgtcaccttttggttgatcgagaatacctactatggcgagaatgtgtacatgaccggcaacgtcagcgctctcggcgactggaacgccagcgcaggctatagtctgaatgccggtctctacacctcggatgagaatctttggttcgcaactgtcaagggactcgagccgggtgttaccatggagtacaagttctacaagattgaaccgggaaactccgtgacttttgaggggggggagaacagggtgtatgcagttcctacagcatgtcctgtggcgcctcaggtccacgctgtttggcagacctga

SEQ ID NO: 33 - protein, 620 aa, Aspergillus
MRFAGIAVGIASVLGHLATPSSAKELAARSPAAANSRLVKEGLFAYESILAALGNTGINAPGTAAGLLIASPTIQNPDYFYTWTRDAALTFKGLVDIFIGGDTFIVVNLDGLETHIQDYISSQAVLQNVSNPSGRLSDGSGLGEPKFEVNFNPYSGGWGRPQRDGPALRAITMLTYIRQLIQQGKQSVASNLIWPVVANDLTYVAQYWNHTGFDLWEEIDGSSFFTTAVQHRAMVEGSAIAQALGKPHAGYDAVAPEILCLLQSYWNESAIISNINVNNGRSGIDLNSVLTSIHTFDPAAGCDDSTFQQCSSKALANHKVYVDSFRSIYGINAGLGPGKAANVGRYAEDIYQGGNPWYLATLAAAEQLYDALYQWKKQGYLTVTQTSLAFFRDFSSTVEPGTYKSNTPNYKSLTEYVRTYADDFFFLVEKYTPSNGSLAEQYDRNTGVPLSANDLTWSYAAFLSTLQRRLNIMPDSWGPSSANTVPTTCSKTTITGTYSAVAPPFPTSTAQCVAAVTVPVTFWLIENTYYGENVYMTGNVSALGDWNASAGYSLNAGLYTSDENLWFATVKGLEPGVTMEYKFYKIEPGNSVTFEGGENRVYAVPTACPVAPQVHAVWQT

SEQ ID NO: 34 - DNA, 1943 bp, Aspergillus
atgcatatccttccaggatcccaacatgcggcagagttggacaacagtggaactctaatacactctgtccattgcgatccagaacagaaagccaagaatatcccgcaatcgacgggtatagctcaagcgagctctgaatggcgaccgtcataccatcttgctgcgccccgggggtggatgaatgacccctgtgggcttgggtatgacccgacgactggactgtaccacctgtccttccaatggaacccgcatgggaatgactggggcaacatctcctggggccatgccacctccagcgatctagtgtcctggcaaatatcccccgaaccatgtctcacgccgtctgcggagtatgatcgatgcggcgtgttcactggctgcttccgttctcatggtccagatggcaagccaggtgtactaacgtatgtatacacatccgtaaatcaccttccactccactatactctgccgtatgtcaagggcagtgaatcgctcagtatagctgtctcccgtgaccatggcaagacctggcaacgaatcgacagtaatccgattcaccccggcgcccctgcaggactcgaggtgacaggctggagagacccttatctgaactgctggccctcgttgcgggcccagcgacagggtggagtggcctccccggatctgtatggatttatctccgggggcatcgctaaagagtccccaacagtctttgtatatgtcgtcaatcccgacaatctgactgagtggacatatatcgggcctttgcttcatgttggcttgaattatcgaccctctcgatggtcgggggatctcggcgtcaactgggaagttgccaacttctttacgttgacagatggcggcgtctcccgggatatcgtgatcttcggcgcagagggatgcttgtcttgcgaggttggatcaaagagagtaccgcgctctttgctctggatgtgcattaatgtccgacctggactgcaggcgcaaagcagtggcgagcctctcgcagattattcattctcggggatcttcgatcacgggtgctgctacgctgcaaactctttctgggacccggtcacggaggagtatgtcgtatactgctggattacagaggaggatttaccagatcgcctgcgccatcgacaaggctggagtggcatcatgtctctaccgcgactagtaaggctggtgactcttcacaatgtcaagcgagcccaccagtcaaagctggaatccattacgtctgtcgaaatagagcgccattcgcaaggtactcaggtccgaaccttgagtgtccggcctgaccccaggctcaacatacttcgaacatcggctcgagagctgcatttatccaatgtacaactgggctctgtggcccatcagcccccggcattcttgcccctgcgcactgctaggtgggaaatgaccgcgactttcgtgattgggacgcattgcgcggcagtaggattggaaatcggccatagccctggtgagctctcacgcttcttcttttcccaaaattgcaacaaatgctgacgcacaaagaagatttccaccagcgcaccaccctatcatggataccatacgacgagacgttcacaatcgaacggcctccgcttcacgatgccggtatcaaccatgtccccgagaccgctccgcatactctcttcaccttctgcaacaatgaaggcgaggaggtgacggaaccgcttcagatccacgcctatttcgatgcaagtgtgctagaagtgtttgtgaatagcaggacagtcatctccacacgcatctatacaccccacgcgcaggtctgcacggggctgaaattcttcgcctcggcaaccgaaagtcagccgaaaccatcaacatctgctcctgctgctgtgctggtgagagcagatatttgggatggactgagtgtgattcgtgatgaaattaagcattag

SEQ ID NO: 35 - DNA, 1887 bp, Aspergillus
  CDS (1)...(1887)
atgcatatccttccaggatcccaacatgcggcagagttggacaacagtggaactctaatacactctgtccattgcgatccagaacagaaagccaagaatatcccgcaatcgacgggtatagctcaagcgagctctgaatggcgaccgtcataccatcttgctgcgccccgggggtggatgaatgacccctgtgggcttgggtatgacccgacgactggactgtaccacctgtccttccaatggaacccgcatgggaatgactggggcaacatctcctggggccatgccacctccagcgatctagtgtcctggcaaatatcccccgaaccatgtctcacgccgtctgcggagtatgatcgatgcggcgtgttcactggctgcttccgttctcatggtccagatggcaagccaggtgtactaacgtatgtatacacatccgtaaatcaccttccactccactatactctgccgtatgtcaagggcagtgaatcgctcagtatagctgtctcccgtgaccatggcaagacctggcaacgaatcgacagtaatccgattcaccccggcgcccctgcaggactcgaggtgacaggctggagagacccttatctgaactgctggccctcgttgcgggcccagcgacagggtggagtggcctccccggatctgtatggatttatctccgggggcatcgctaaagagtccccaacagtctttgtatatgtcgtcaatcccgacaatctgactgagtggacatatatcgggcctttgcttcatgttggcttgaattatcgaccctctcgatggtcgggggatctcggcgtcaactgggaagttgccaacttctttacgttgacagatggcggcgtctcccgggatatcgtgatcttcggcgcagagggatgcttgtcttgcgaggttggatcaaagagagtaccgcgctctttgctctggatgtgcattaatgtccgacctggactgcaggcgcaaagcagtggcgagcctctcgcagattattcattctcggggatcttcgatcacgggtgctgctacgctgcaaactctttctgggacccggtcacggaggagtatgtcgtatactgctggattacagaggaggatttaccagatcgcctgcgccatcgacaaggctggagtggcatcatgtctctaccgcgactagtaaggctggtgactcttcacaatgtcaagcgagcccaccagtcaaagctggaatccattacgtctgtcgaaatagagcgccattcgcaaggtactcaggtccgaaccttgagtgtccggcctgaccccaggctcaacatacttcgaacatcggctcgagagctgcatttatccaatgtacaactgggctctgtggcccatcagcccccggcattcttgcccctgcgcactgctaggtgggaaatgaccgcgactttcgtgattgggacgcattgcgcggcagtaggattggaaatcggccatagccctgaagatttccaccagcgcaccaccctatcatggataccatacgacgagacgttcacaatcgaacggcctccgcttcacgatgccggtatcaaccatgtccccgagaccgctccgcatactctcttcaccttctgcaacaatgaaggcgaggaggtgacggaaccgcttcagatccacgcctatttcgatgcaagtgtgctagaagtgtttgtgaatagcaggacagtcatctccacacgcatctatacaccccacgcgcaggtctgcacggggctgaaattcttcgcctcggcaaccgaaagtcagccgaaaccatcaacatctgctcctgctgctgtgctggtgagagcagatatttgggatggactgagtgtgattcgtgatgaaattaagcattag

SEQ ID NO: 36 - protein, 628 aa, Aspergillus
MHILPGSQHAAELDNSGTLIHSVHCDPEQKAKNIPQSTGIAQASSEWRPSYHLAAPRGWMNDPCGLGYDPTTGLYHLSFQWNPHGNDWGNISWGHATSSDLVSWQISPEPCLTPSAEYDRCGVFTGCFRSHGPDGKPGVLTYVYTSVNHLPLHYTLPYVKGSESLSIAVSRDHGKTWQRIDSNPIHPGAPAGLEVTGWRDPYLNCWPSLRAQRQGGVASPDLYGFISGGIAKESPTVFVYVVNPDNLTEWTYIGPLLHVGLNYRPSRWSGDLGVNWEVANFFTLTDGGVSRDIVIFGAEGCLSCEVGSKRVPRSLLWMCINVRPGLQAQSSGEPLADYSFSGIFDHGCCYAANSFWDPVTEEYVVYCWITEEDLPDRLRHRQGWSGIMSLPRLVRLVTLHNVKRAHQSKLESITSVEIERHSQGTQVRTLSVRPDPRLNILRTSARELHLSNVQLGSVAHQPPAFLPLRTARWEMTATFVIGTHCAAVGLEIGHSPEDFHQRTTLSWIPYDETFTIERPPLHDAGINHVPETAPHTLFTFCNNEGEEVTEPLQIHAYFDASVLEVFVNSRTVISTRIYTPHAQVCTGLKFFASATESQPKPSTSAPAAVLVRADIWDGLSVIRDEIKH

SEQ ID NO: 37 - DNA, 1050 bp, Aspergillus
atggttcactttaagtctgtctgtacgctggctgttacggcgtttgctgcgctgggtgctgcggcgccagcaatgttggcaaagcgaggtatgtctgacgcttcctacgattgggctatgtgcgatgctaactattaagtagacgtgtcctcgtcggtgctgcaaaaactgtcactgtttgcgcaatactctgctgcctcctattgtaccaacaacatcaattccacgggcaacaagctgacatgctcctctggagagtgcccgctggtcgaggcagccaacaccaagaccctttcggaattctacgagtaggtcgatcccatgcatgagtagctcgtatctctaacagagttggtagtgattccgcgtatggagacgtcgcaggcttcttggttgcagacaccacgaacaagctacttgtggtctctttcagaggaagccgcacgatagacacatggttggcgaacctggactttggcctggacagtatcagtgatgtttgcagcggatgtgcggtacataagggattctggaagtcctgggaagtcgttgccaatgcactaacgaccgagctaaactctgcccttgcaacttacagtggctataccgttgtctttactggccatagcttcggcgctgctcttgcaacgctgggggctgctacgttgcggaaagcagggattcccgtagagctggtaagtcatcccttgtcaaatcaggtaagggcgtaatgggactaattgattgaagtatggttacggatccccgcgtgttggaaataaggccttggcaacattcatcaccggacagggttccaattaccgtgtcacacacacaaacgacattgtccccagactcccgccccgagtctttggcttcagccacattagcccagagtactggatcacgagcggtgacaacgctcctgtcacgacgtctgatgtcacggttgtccagggaatcgactcaagcggtggaaatgcgggcgaggattctaccagcattgaggcccataattggtatattggccatattgatggttgtcaataa

SEQ ID NO: 38 - DNA, 900 bp, Aspergillus
  CDS (1)...(900)
atggttcactttaagtctgtctgtacgctggctgttacggcgtttgctgcgctgggtgctgcggcgccagcaatgttggcaaagcgagacgtgtcctcgtcggtgctgcaaaaactgtcactgtttgcgcaatactctgctgcctcctattgtaccaacaacatcaattccacgggcaacaagctgacatgctcctctggagagtgcccgctggtcgaggcagccaacaccaagaccctttcggaattctacgaagttggtagtgattccgcgtatggagacgtcgcaggcttcttggttgcagacaccacgaacaagctacttgtggtctctttcagaggaagccgcacgatagacacatggttggcgaacctggactttggcctggacagtatcagtgatgtttgcagcggatgtgcggtacataagggattctggaagtcctgggaagtcgttgccaatgcactaacgaccgagctaaactctgcccttgcaacttacagtggctataccgttgtctttactggccatagcttcggcgctgctcttgcaacgctgggggctgctacgttgcggaaagcagggattcccgtagagctgtatggttacggatccccgcgtgttggaaataaggccttggcaacattcatcaccggacagggttccaattaccgtgtcacacacacaaacgacattgtccccagactcccgccccgagtctttggcttcagccacattagcccagagtactggatcacgagcggtgacaacgctcctgtcacgacgtctgatgtcacggttgtccagggaatcgactcaagcggtggaaatgcgggcgaggattctaccagcattgaggcccataattggtatattggccatattgatggttgtcaataa

SEQ ID NO: 39 - protein, 299 aa, Aspergillus
MVHFKSVCTLAVTAFAALGAAAPAMLAKRDVSSSVLQKLSLFAQYSAASYCTNNINSTGNKLTCSSGECPLVEAANTKTLSEFYEVGSDSAYGDVAGFLVADTTNKLLVVSFRGSRTIDTWLANLDFGLDSISDVCSGCAVHKGFWKSWEVVANALTTELNSALATYSGYTVVFTGHSFGAALATLGAATLRKAGIPVELYGYGSPRVGNKALATFITGQGSNYRVTHTNDIVPRLPPRVFGFSHISPEYWITSGDNAPVTTSDVTVVQGIDSSGGNAGEDSTSIEAHNWYIGHIDGCQ

SEQ ID NO: 40 - DNA, 1926 bp, Aspergillus
atgcatatacgctggtcatccttttttctttcttgccttgctggaacagctttggcggccactccagcgcaatggcgatcccagtccatctatttcttactcacggatcgattcgcaaggactgacggttccacaacggcctcgtgtgatactagtgctcgagtcagctacacgagcctcccttcacaagccacagtgtactgacacgtgaaataggagtattgtggtggtacatggcaaggcattattgaacaagtcagaactagtgtggagagtaagagtgtaacaatggctgattgggtcaatatagctcgattacatacaaggcatgggctttacagcaatttggataaccccagtcaccaagcaacttccccaggatacgtcggaaggcactgcataccacgggtactggcagcaagacatgtgagcatcaagtcatccccacagtctcgtattagctttttgctgactcggtaagttattcggtcaattccaactatgggaccgctgacgacctgaaagccctggcgtcagctcttcatgacaggggcatgtatctcatggtcgatgttgttgccaatcacatggttcgctggatcagcccattgatgtgctgcttgctgattgaaccagggatatgccggtgcaggcgattcggtcgactatagtgtcttcaaccccttcaactcccagacctccttccacccattgtgcttcatcagcaattacgataatcagacagacgtggaaaattgctggttgggggacaattcggttcctttacccgatcttgatactacaaatccggatgttcaaaagatttggtacaattgggtgaactctttagtgtctaattattccagtaaggacggactcttggtatctgcacaaaaacaagaattgactgatatgctacagtcgacggcctacgaattgacactgtgaagcacgtccagagcgatttctggccgggattcaatgacgccgcaggcgtctactgtatcggggaggtatttgatggagatccagcgtacacttgtccctaccaagaggtcctggacggggtgctgaattaccctatgtaagtcgcataagcatatgaacctgtccatggctgcggatctaattagagcagatattatccgcttctgaaagccttccaatccacaagtggcagtatgagcagcctctacgatatgatcaacacggtcaagtcgcagtgcgccgattcgacgctgttgggcacctttgtcgaaaatcacgatactccccggttcgcatcgtaagtttcttcctagactcattgtcattccagcgtcctgatgaatccccgctcgcaggtataccaaggacatggccctcgctaaaaacgccgcagcattcatcatcttctccgatggaatcccaatcatctacgccggccaggagcaacattacagcggcggcgcggatcccgcaaaccgtgaggcagtctggctatccggctactcgacaacgagtgacctatacaagctcatcgcgacagcaaacgccatccggagtcacgccattagcaaggacccgggatacgtgacttataaggtacgaggctctcactcacggaaggaccatacgatcaagcagggcttactcaacccacagaataaccccatctacaaagatacctccaccatcgccatgcgaaaaggctccgacggagcgcagatcatcaccgtcctctcgaacctcggtgcttctggaagctcttacacgctctcattgggtggaacaggctatgaggccggacaacaactgactgagatgttctcctgcaccacggtgaccgtgggctcagacaaaaaggtccccgtttccatggctagtggcttgccccgggtgttttacccaacggctgggctgaacggaagtactgtttgtacttga

SEQ ID NO: 41 - DNA, 1488 bp, Aspergillus
  CDS (1)...(1488)
atgcatatacgctggtcatccttttttctttcttgccttgctggaacagctttggcggccactccagcgcaatggcgatcccagtccatctatttcttactcacggatcgattcgcaaggactgacggttccacaacggcctcgtgtgatactagtgctcgagagtattgtggtggtacatggcaaggcattattgaacaactcgattacatacaaggcatgggctttacagcaatttggataaccccagtcaccaagcaacttccccaggatacgtcggaaggcactgcataccacgggtactggcagcaagacatttattcggtcaattccaactatgggaccgctgacgacctgaaagccctggcgtcagctcttcatgacaggggcatgtatctcatggtcgatgttgttgccaatcacatgggatatgccggtgcaggcgattcggtcgactatagtgtcttcaaccccttcaactcccagacctccttccacccattgtgcttcatcagcaattacgataatcagacagacgtggaaaattgctggttgggggacaattcggttcctttacccgatcttgatactacaaatccggatgttcaaaagatttggtacaattgggtgaactctttagtgtctaattattccatcgacggcctacgaattgacactgtgaagcacgtccagagcgatttctggccgggattcaatgacgccgcaggcgtctactgtatcggggaggtatttgatggagatccagcgtacacttgtccctaccaagaggtcctggacggggtgctgaattaccctatatattatccgcttctgaaagccttccaatccacaagtggcagtatgagcagcctctacgatatgatcaacacggtcaagtcgcagtgcgccgattcgacgctgttgggcacctttgtcgaaaatcacgatactccccggttcgcatcgtataccaaggacatggccctcgctaaaaacgccgcagcattcatcatcttctccgatggaatcccaatcatctacgccggccaggagcaacattacagcggcggcgcggatcccgcaaaccgtgaggcagtctggctatccggctactcgacaacgagtgacctatacaagctcatcgcgacagcaaacgccatccggagtcacgccattagcaaggacccgggatacgtgacttataagaataaccccatctacaaagatacctccaccatcgccatgcgaaaaggctccgacggagcgcagatcatcaccgtcctctcgaacctcggtgcttctggaagctcttacacgctctcattgggtggaacaggctatgaggccggacaacaactgactgagatgttctcctgcaccacggtgaccgtgggctcagacaaaaaggtccccgtttccatggctagtggcttgccccgggtgttttacccaacggctgggctgaacggaagtactgtttgtacttga

SEQ ID NO: 42 - protein, 495 aa, Aspergillus
MHIRWSSFFLSCLAGTALAATPAQWRSQSIYFLLTDRFARTDGSTTASCDTSAREYCGGTWQGIIEQLDYIQGMGFTAIWITPVTKQLPQDTSEGTAYHGYWQQDIYSVNSNYGTADDLKALASALHDRGMYLMVDVVANHMGYAGAGDSVDYSVFNPFNSQTSFHPLCFISNYDNQTDVENCWLGDNSVPLPDLDTTNPDVQKIWYNWVNSLVSNYSIDGLRIDTVKHVQSDFWPGFNDAAGVYCIGEVFDGDPAYTCPYQEVLDGVLNYPIYYPLLKAFQSTSGSMSSLYDMINTVKSQCADSTLLGTFVENHDTPRFASYTKDMALAKNAAAFIIFSDGIPIIYAGQEQHYSGGADPANREAVWLSGYSTTSDLYKLIATANAIRSHAISKDPGYVTYKNNPIYKDTSTIAMRKGSDGAQIITVLSNLGASGSSYTLSLGGTGYEAGQQLTEMFSCTTVTVGSDKKVPVSMASGLPRVFYPTAGLNGSTVCT

SEQ ID NO: 43 - DNA, 2250 bp, Aspergillus
atgaagtggatctcgcagctcttcccgttgtccctgtgctcgtccctgctcggacaggctgcccatgctctgaccccagccgaatggcgcagtcaatcgatctatttcctcctgaccgatcggttcggccgagaagacaattccacgactgctgcctgcgatgtcacgcaacgagtgtgtccccctatcgttcttgatcgagctcatgctaacatttgcagctgtattgcggcgggagctggcaggggatcatcaatcatgtacgactgtcccatatcgcgcggtcgaaactaacaatgtcagctcgactacattcaaggcatgggatttactgccatatggatcacccccgtaactgagcagttctatgagaacaccggcgatggtacttcgtaccatggatactggcagcagaatatgtgagttctctttggccgcggtgattcatactaatcgacgaagccacgaggtcaatgccaattatggaacggcacaagatcttagagatctggccaacgctctgcacgcgcgtggcatgtacttgatggtcgatgtggtcgccaaccatatggtacgccgtcttctcgagttgtacattataatctgactggttaagggctacaacggagcgggaaactcggtcaactacggtgtcttcactccgtttgattccgctacctatttccacccatactgtctcatcaccgactacaacaaccaaacagctgtggaggactgctggctgggagatactactgtctcgctacccgatctcgacacgaccagcacggcagtgcgaagcatctggtatgattgggtgaagggattggttgccaactactccagtcagtgagagccgcatccgagcgtgaatgatactgaccgagatagtcgacggcctgcgcatcgacacggtgaagcatgtcgagaaagacttctggcccggctacaatgacgctgctggcgtctactgtgtcggtgaagtcttttcgggtgatccacaatatacctgtccataccagaattacctggatggtgtactcaactaccccatgtacgcattatgatttccgtacggatttgatcctgacgagagcagatactatcaacttctctacgcgttccaatcgaccagcggcagcatcagcaatctgtacaacatgatcagctccgttgcgtctgactgtgcggatcccactttgctcggcaactttatcgagaaccatgataacccccgatttgcctcgtaagaccgtgcctccgttccctgaatgcaactaacctccccagctatacgagcgactattcgcaagccaagaacgtcatctccttcatgttcttctccgacggcatccccattgtctacgccggacaggagcagcactacagcggcggtgctgaccctgccaaccgcgaggctgtctggctgtctggatactcgaccagcgctacgctgtacagctggattgcctctaccaacaagattcgcaagctagcgatttccaaagactcagcctacataacatccaaggtatttccggtcacgtcttcgcattccaccgctaacatcgatagaacaacccgttctactatgattccaatactctcgctatgcgcaagggctcagtcgctggctctcaagtcattaccgtcctcagtaacaagggatcctcgggcagttcctacaccctctctctcagcggcacgggctactccgccggcgccacccttgtcgagatgtatacatgcactactctcaccgtggactcgagcggaaatctcgccgtgccaatggtatccggcttgcccagagttttcgtgccctcgtcatgggtcagtgggagtggcctctgcggcgactctatctccaccacggcgaccgcccccagtgccaccacgagcgcaacagcgacaagaacagcatgcgcagctgccacagccattccgattctcttcgaggagctcgtgacaactacctacggcgagtccatctacctgaccggctcgatcagccaactcgggaactgggacacgagttctgcgattgctctgtcggcgagtaaatacacctcgtcgaaccctgagtggtatgtcaccgtgaccctgcctgttggcacctcatttgagtacaaattcgtcaagaaggggtcggatgggagcatcgcgtgggaaagtgatccgaaccggtcgtatacggtgccgactgggtgtgcgggaacgaccgtgacggtgtctgatacgtggagatga

SEQ ID NO: 44 - DNA, 1893 bp, Aspergillus
  CDS (1)...(1893)
atgaagtggatctcgcagctcttcccgttgtccctgtgctcgtccctgctcggacaggctgcccatgctctgaccccagccgaatggcgcagtcaatcgatctatttcctcctgaccgatcggttcggccgagaagacaattccacgactgctgcctgcgatgtcacgcaacgactgtattgcggcgggagctggcaggggatcatcaatcatctcgactacattcaaggcatgggatttactgccatatggatcacccccgtaactgagcagttctatgagaacaccggcgatggtacttcgtaccatggatactggcagcagaatatccacgaggtcaatgccaattatggaacggcacaagatcttagagatctggccaacgctctgcacgcgcgtggcatgtacttgatggtcgatgtggtcgccaaccatatgggctacaacggagcgggaaactcggtcaactacggtgtcttcactccgtttgattccgctacctatttccacccatactgtctcatcaccgactacaacaaccaaacagctgtggaggactgctggctgggagatactactgtctcgctacccgatctcgacacgaccagcacggcagtgcgaagcatctggtatgattgggtgaagggattggttgccaactactccatcgacggcctgcgcatcgacacggtgaagcatgtcgagaaagacttctggcccggctacaatgacgctgctggcgtctactgtgtcggtgaagtcttttcgggtgatccacaatatacctgtccataccagaattacctggatggtgtactcaactaccccatatactatcaacttctctacgcgttccaatcgaccagcggcagcatcagcaatctgtacaacatgatcagctccgttgcgtctgactgtgcggatcccactttgctcggcaactttatcgagaaccatgataacccccgatttgcctcctatacgagcgactattcgcaagccaagaacgtcatctccttcatgttcttctccgacggcatccccattgtctacgccggacaggagcagcactacagcggcggtgctgaccctgccaaccgcgaggctgtctggctgtctggatactcgaccagcgctacgctgtacagctggattgcctctaccaacaagattcgcaagctagcgatttccaaagactcagcctacataacatccaagaacaacccgttctactatgattccaatactctcgctatgcgcaagggctcagtcgctggctctcaagtcattaccgtcctcagtaacaagggatcctcgggcagttcctacaccctctctctcagcggcacgggctactccgccggcgccacccttgtcgagatgtatacatgcactactctcaccgtggactcgagcggaaatctcgccgtgccaatggtatccggcttgcccagagttttcgtgccctcgtcatgggtcagtgggagtggcctctgcggcgactctatctccaccacggcgaccgcccccagtgccaccacgagcgcaacagcgacaagaacagcatgcgcagctgccacagccattccgattctcttcgaggagctcgtgacaactacctacggcgagtccatctacctgaccggctcgatcagccaactcgggaactgggacacgagttctgcgattgctctgtcggcgagtaaatacacctcgtcgaaccctgagtggtatgtcaccgtgaccctgcctgttggcacctcatttgagtacaaattcgtcaagaaggggtcggatgggagcatcgcgtgggaaagtgatccgaaccggtcgtatacggtgccgactgggtgtgcgggaacgaccgtgacggtgtctgatacgtggagatga

SEQ ID NO: 45 - protein, 630 aa, Aspergillus
MKWISQLFPLSLCSSLLGQAAHALTPAEWRSQSIYFLLTDRFGREDNSTTAACDVTQRLYCGGSWQGIINHLDYIQGMGFTAIWITPVTEQFYENTGDGTSYHGYWQQNIHEVNANYGTAQDLRDLANALHARGMYLMVDVVANHMGYNGAGNSVNYGVFTPFDSATYFHPYCLITDYNNQTAVEDCWLGDTTVSLPDLDTTSTAVRSIWYDWVKGLVANYSIDGLRIDTVKHVEKDFWPGYNDAAGVYCVGEVFSGDPQYTCPYQNYLDGVLNYPIYYQLLYAFQSTSGSISNLYNMISSVASDCADPTLLGNFIENHDNPRFASYTSDYSQAKNVISFMFFSDGIPIVYAGQEQHYSGGADPANREAVWLSGYSTSATLYSWIASTNKIRKLAISKDSAYITSKNNPFYYDSNTLAMRKGSVAGSQVITVLSNKGSSGSSYTLSLSGTGYSAGATLVEMYTCTTLTVDSSGNLAVPMVSGLPRVFVPSSWVSGSGLCGDSISTTATAPSATTSATATRTACAAATAIPILFEELVTTTYGESIYLTGSISQLGNWDTSSAIALSASKYTSSNPEWYVTVTLPVGTSFEYKFVKKGSDGSIAWESDPNRSYTVPTGCAGTTVTVSDTWR

SEQ ID NO: 46 - DNA, 1707 bp, Aspergillus
atgatatactcgctgcgaatcctgcagtggctttgcatcactgcagggcttgacctgctcgtgccatccttggctgcagatacaagtgcatggaagtccaggtctatctatcagacgatgacagacagatttgcacgtacggatgggtccaccactcatccatgcaacaccacagaagggctacgctgtggtggctcatggagaggtacaatccaacacctcgattacattcaaggaatgggattcgacgctatcatgatctccccaatcgtccagaatgtcgaggggcgcgtgcagtacggagaggcctatcatggctactgggtccaggacatgtatgcgctaaacccacactttggcactcaccaggacctgttagacttgagtaaggctgttcatgatcggggcatgtatctgatggtagatactgtcatcaacaacttggcgtacatcaccgacggacgaaacccggccacgagcattgactattcagcgctcagaccattcaatgactcaatgttctttcatccatattgcaagatcaccgactatgacaactatccattggcgcaaacatgctggacaggtgatgatgttgttccccttccagatctaaagacagaagatagccaagttcagagcatgctcatggactggattagaaaaatgatggctacgtattcaatcgatggcctacgtctggatgctgcgaagcatattacaccaagtttcttgcctttgttccaaaacgcttctggcgcttttattactggagaagttttcgagccgtccgtgaagacgatctgcggatatcaaaaggacctccccagcgtccccaattatcccatctattattccatcttggaggcatttacgaaaggcaacacaagctctctgacgaatcaggtagaggtcatgaaacagacatgttctgatgttactgcccttacttcgttctcggagaatcatgacgtcgctcgatttgcaagcttcaaggatgacatcgcggtaaggcagcgaactccagttaccctgaaagtatcaaccgatgataggctaacaccggcgcagctcgctaagaacgttttaacatttacgatgctgttcgacggcataccgatgatataccaagggcaagaacaacatttctcaggcgccagtgacccggagaaccgcgaagccttatggctatcaggctacaacaccaacgctccgctttaccaactagccacgagactgaacaggatccgcaagcatgcagcccagatcgacccagcatttgtagactcacagaccttccccatctacacagggtctagcgagatgggtttccgcaaaggcgtcgagggccggcaactcgtaatggtcttgtcaactcaaggtatggacagcggcaaatatgagctggacatgccaataggctaccaacctggatttgtggtcacagatgtgcttaattgcgccaactattcggttaacgacatgggtcttctcaaggtcagcatggataagggcgagcctcgggtcctttttcccgctgacatgatggaaggaagtggtctatgcggatatggaaggtcgaatggaacgacgtatcttaatctgaaaaagaattcaacgcagttctcgggtggcgggctctcaaatcggcctaccgtgcccctactgttgatctgttgtttgacttgcttgatactatggtgttga

SEQ ID NO: 47 - DNA, 1644 bp, Aspergillus
  CDS (1)...(1644)
atgatatactcgctgcgaatcctgcagtggctttgcatcactgcagggcttgacctgctcgtgccatccttggctgcagatacaagtgcatggaagtccaggtctatctatcagacgatgacagacagatttgcacgtacggatgggtccaccactcatccatgcaacaccacagaagggctacgctgtggtggctcatggagaggtacaatccaacacctcgattacattcaaggaatgggattcgacgctatcatgatctccccaatcgtccagaatgtcgaggggcgcgtgcagtacggagaggcctatcatggctactgggtccaggacatgtatgcgctaaacccacactttggcactcaccaggacctgttagacttgagtaaggctgttcatgatcggggcatgtatctgatggtagatactgtcatcaacaacttggcgtacatcaccgacggacgaaacccggccacgagcattgactattcagcgctcagaccattcaatgactcaatgttctttcatccatattgcaagatcaccgactatgacaactatccattggcgcaaacatgctggacaggtgatgatgttgttccccttccagatctaaagacagaagatagccaagttcagagcatgctcatggactggattagaaaaatgatggctacgtattcaatcgatggcctacgtctggatgctgcgaagcatattacaccaagtttcttgcctttgttccaaaacgcttctggcgcttttattactggagaagttttcgagccgtccgtgaagacgatctgcggatatcaaaaggacctccccagcgtccccaattatcccatctattattccatcttggaggcatttacgaaaggcaacacaagctctctgacgaatcaggtagaggtcatgaaacagacatgttctgatgttactgcccttacttcgttctcggagaatcatgacgtcgctcgatttgcaagcttcaaggatgacatcgcgctcgctaagaacgttttaacatttacgatgctgttcgacggcataccgatgatataccaagggcaagaacaacatttctcaggcgccagtgacccggagaaccgcgaagccttatggctatcaggctacaacaccaacgctccgctttaccaactagccacgagactgaacaggatccgcaagcatgcagcccagatcgacccagcatttgtagactcacagaccttccccatctacacagggtctagcgagatgggtttccgcaaaggcgtcgagggccggcaactcgtaatggtcttgtcaactcaaggtatggacagcggcaaatatgagctggacatgccaataggctaccaacctggatttgtggtcacagatgtgcttaattgcgccaactattcggttaacgacatgggtcttctcaaggtcagcatggataagggcgagcctcgggtcctttttcccgctgacatgatggaaggaagtggtctatgcggatatggaaggtcgaatggaacgacgtatcttaatctgaaaaagaattcaacgcagttctcgggtggcgggctctcaaatcggcctaccgtgcccctactgttgatctgttgtttgacttgcttgatactatggtgttga

SEQ ID NO: 48 - protein, 547 aa, Aspergillus
MIYSLRILQWLCITAGLDLLVPSLAADTSAWKSRSIYQTMTDRFARTDGSTTHPCNTTEGLRCGGSWRGTIQHLDYIQGMGFDAIMISPIVQNVEGRVQYGEAYHGYWVQDMYALNPHFGTHQDLLDLSKAVHDRGMYLMVDTVINNLAYITDGRNPATSIDYSALRPFNDSMFFHPYCKITDYDNYPLAQTCWTGDDVVPLPDLKTEDSQVQSMLMDWIRKMMATYSIDGLRLDAAKHITPSFLPLFQNASGAFITGEVFEPSVKTICGYQKDLPSVPNYPIYYSILEAFTKGNTSSLTNQVEVMKQTCSDVTALTSFSENHDVARFASFKDDIALAKNVLTFTMLFDGIPMIYQGQEQHFSGASDPENREALWLSGYNTNAPLYQLATRLNRIRKHAAQIDPAFVDSQTFPIYTGSSEMGFRKGVEGRQLVMVLSTQGMDSGKYELDMPIGYQPGFVVTDVLNCANYSVNDMGLLKVSMDKGEPRVLFPADMMEGSGLCGYGRSNGTTYLNLKKNSTQFSGGGLSNRPTVPLLLICCLTCLILWC

SEQ ID NO: 49 - DNA, 1906 bp, Aspergillus
atgggcttctttcgcgatggactgacttccttaggcctgcttctgttatgctttgtttcatggggatcagctggtgtggtgcgcttcaaaatcacactgacatgggaggactggactccgactggtattgcgcggaaaatgattctcaccaatggccaatttccagctccgccattgtacgtgcgacagggcgatgatgtcgagtttttggtagataatcaacttccctttgctaccgcagtccatttccacggtaaagcttgttcactgacagcaattgctagccaaggctgacacctcacctcaatcaggtattgaccaaatggggacgccatggtccgatggagttcctggtctttcgcaaagacccattccgtccggcagcagttttctttacaaatggaatgcgggacagtatggcagctatatgtatcacgctcacagcagagggcagattgatgatggcttatacggagcgatttatatccgtcctggtgacgaagtggagaaacccttttggttgataagtaatcgtacacgtgaagtcaaggccatgcgacgagccgaagaaaggacaacccccatagtgctgagtgactggcgccagttgacgtccgaggagctgtggcatgcggaagaggcaactggactagacgcatactgtgtgaatgcgctattagtcaatggaaggggctccgtacagtgtctagaccgcagtacgttggatcggtatagcgcggcaaagtgggcatttttgggaaactcttcgctgactgatattgggtaagagtgaaaaaaatacggcgattcgacatattgatctgaagctgactctcccagctgcgcgcccccgacgattcctctgctccaaggggactttccacacaactttagtgccacgccaccgacactgttttctggctgtactccgtctcagggatcgacggagctactcctcgttgatccacaagcttcgtacgccagctttgacctgattagtgctgctggtgtgtcgatgcctactttctcaatcgacgaacatcccatgtatatatacgccatcgatggaagatatatcgtcccggttcgagtggatgccatcaccatcggcaatggaaaccgttattcggtcatggtgaaactggataaaccggctggtgattacaccgtgcgtgtagcgaacgccggaatcaaccaacttattactgctaatgcgagcatgtcttacaacacgcttttcagggctcaatcacgtccttcgcagccatcaatcgacatcaccggcgcaaacacaacagcggacgtcgtgatcctcgacgagagcagggtgatccctttccctgtggaggtaccggcgcaagacgttgcccagaccttcttcctagatgtcgcccggttcaacgcgtcctaccggtggattcttggaagctcggacttcccgctctccgtggaggaatcgcccccacttctgttcaatcgctccgccgccaagcccgatctttcgatttcaacgcgtaacggaacttgggtcgatctcatcttcagagtcaccggccctttacagccaccacaccctattcacaagcactcgaacaagttcttcgtcattggtcaagggaacggtgtcttcaattatacctcagtgactgaggcgcggaaacacattccggaaagcttcaatcttaatgccccgcagatacgggacacgttcgcgacgcctcccagtgtatccggccccacttggttggcaataagatatcatgtcgtgaatcctggcgcattcttgatccattgtcacatccaaatccatctgagcggtggtatggcgctggcaatattggacggcgtagataagtggcctgtggacattccccaggagtaccagcttgcggcctcaggatcatag

SEQ ID NO: 50 - DNA, 1791 bp, aspergillus
  CDS (1)...(1791)
atgggcttctttcgcgatggactgacttccttaggcctgcttctgttatgctttgtttcatggggatcagctggtgtggtgcgcttcaaaatcacactgacatgggaggactggactccgactggtattgcgcggaaaatgattctcaccaatggccaatttccagctccgccattgtacgtgcgacagggcgatgatgtcgagtttttggtagataatcaacttccctttgctaccgcagtccatttccacggtattgaccaaatggggacgccatggtccgatggagttcctggtctttcgcaaagacccattccgtccggcagcagttttctttacaaatggaatgcgggacagtatggcagctatatgtatcacgctcacagcagagggcagattgatgatggcttatacggagcgatttatatccgtcctggtgacgaagtggagaaacccttttggttgataagtaatcgtacacgtgaagtcaaggccatgcgacgagccgaagaaaggacaacccccatagtgctgagtgactggcgccagttgacgtccgaggagctgtggcatgcggaagaggcaactggactagacgcatactgtgtgaatgcgctattagtcaatggaaggggctccgtacagtgtctagaccgcagtacgttggatcggtatagcgcggcaaagtgggcatttttgggaaactcttcgctgactgatattggctgcgcgcccccgacgattcctctgctccaaggggactttccacacaactttagtgccacgccaccgacactgttttctggctgtactccgtctcagggatcgacggagctactcctcgttgatccacaagcttcgtacgccagctttgacctgattagtgctgctggtgtgtcgatgcctactttctcaatcgacgaacatcccatgtatatatacgccatcgatggaagatatatcgtcccggttcgagtggatgccatcaccatcggcaatggaaaccgttattcggtcatggtgaaactggataaaccggctggtgattacaccgtgcgtgtagcgaacgccggaatcaaccaacttattactgctaatgcgagcatgtcttacaacacgcttttcagggctcaatcacgtccttcgcagccatcaatcgacatcaccggcgcaaacacaacagcggacgtcgtgatcctcgacgagagcagggtgatccctttccctgtggaggtaccggcgcaagacgttgcccagaccttcttcctagatgtcgcccggttcaacgcgtcctaccggtggattcttggaagctcggacttcccgctctccgtggaggaatcgcccccacttctgttcaatcgctccgccgccaagcccgatctttcgatttcaacgcgtaacggaacttgggtcgatctcatcttcagagtcaccggccctttacagccaccacaccctattcacaagcactcgaacaagttcttcgtcattggtcaagggaacggtgtcttcaattatacctcagtgactgaggcgcggaaacacattccggaaagcttcaatcttaatgccccgcagatacgggacacgttcgcgacgcctcccagtgtatccggccccacttggttggcaataagatatcatgtcgtgaatcctggcgcattcttgatccattgtcacatccaaatccatctgagcggtggtatggcgctggcaatattggacggcgtagataagtggcctgtggacattccccaggagtaccagcttgcggcctcaggatcatag

SEQ ID NO: 51 - protein, 596 aa, aspergillus
MGFFRDGLTSLGLLLLCFVSWGSAGVVRFKITLTWEDWTPTGIARKMILTNGQFPAPPLYVRQGDDVEFLVDNQLPFATAVHFHGIDQMGTPWSDGVPGLSQRPIPSGSSFLYKWNAGQYGSYMYHAHSRGQIDDGLYGAIYIRPGDEVEKPFWLISNRTREVKAMRRAEERTTPIVLSDWRQLTSEELWHAEEATGLDAYCVNALLVNGRGSVQCLDRSTLDRYSAAKWAFLGNSSLTDIGCAPPTIPLLQGDFPHNFSATPPTLFSGCTPSQGSTELLLVDPQASYASFDLISAAGVSMPTFSIDEHPMYIYAIDGRYIVPVRVDAITIGNGNRYSVMVKLDKPAGDYTVRVANAGINQLITANASMSYNTLFRAQSRPSQPSIDITGANTTADVVILDESRVIPFPVEVPAQDVAQTFFLDVARFNASYRWILGSSDFPLSVEESPPLLFNRSAAKPDLSISTRNGTWVDLIFRVTGPLQPPHPIHKHSNKFFVIGQGNGVFNYTSVTEARKHIPESFNLNAPQIRDTFATPPSVSGPTWLAIRYHVVNPGAFLIHCHIQIHLSGGMALAILDGVDKWPVDIPQEYQLAASGS

SEQ ID NO: 52 - DNA, 2060 bp, Aspergillus
atgcctcgcctttcctacgcgctctgtgcgctgtctctcgggcatgctgctattgcagctcctcagttatccgctcgtgctaccggcagcttggactcctggttgggtactgagaccaccgttgcgctcaatggtattctggccaacatcggtgccgacggtgcttatgcgaagagcgctaagcctggcataatcattgccagtccgagcaccagcgaaccagactgtgagaaccttcctgaactggccctgtccggcagtcattgacctcggtagactactatacctggacgagagatgctgctctcgtcacgaaagtcctggtcgacctcttccgcaacggcaacctgggtctgcagaaagtcattaccgaatacgtcaactctcaggcgtacttgcagaccgtgtctaatccgtcgggtggtcttgcgagcggaggtctcgcggagcctaagtacaacgtcgacatgacggcctttaccggagcctggggtcgtcctcagcgtgatggtccggctctgcgggccaccgccctcatcgactttggcaactggctgattgtatgttctccatacgagccccaggaagcgttgctgacgtctacaggacaacggctactccagctatgctgtcaacaacatctggcccattgtgcgcaacgacttgtcctacgtttctcagtactggagccagagtggctttggtgagtcccgactctctggaagtttacaacgtgcatcgattactgacaattgagattctacgtgacagatctctgggaagaagtcaactccatgtccttcttcaccgtcgctgtccagcaccgtgccctcgtggagggaagcacgttcgctaaacgggtgggagcgtcgtgctcgtggtgtgactcgcaggccccccagatcctctgctacatgcagagtttctggactggctcgtatatcaacgccaacaccggtggtggccggtccggcaaggatgccaacaccgtcctcgccagcatccataccttcgaccccgaagccggctgcgacgatactactttccagccctgctctcctcgggcccttgccaaccacaaggtgtacaccgattcgttccgctcggtctacgcgatcaactccggcatcccacagggcgctgccgtttccgctggccgctaccccgaggacgtctactacaacggcaacccttggttcctcaccaccctcgccgctgccgagcagctctacgacgctatctaccagtggaagaagatcggttccatcagcatcaccagcacctccctcgccttcttcaaggacatctacagctccgccgcggtcggcacctacgcctctagcacctccaccttcacggacatcatcaacgcggtcaagacctacgcagacggctacgtgagcatcgtccaggcacacgccatgaacaacggctccctttcggagcaattcgacaagtcctctgggctgtccctctccgcccgcgatctgacctggtcctacgccgctttcctcaccgccaacatgcgtcgtaacggcgtggtgcctgccccctggggcgccgcctccgccaactccgtcccctcgtcttgctccatgggctcggccacgggcacctacagcaccgcgacagccacctcctggcccagcacgctgaccagcggctcgccaggcagcaccaccaccgtgggcaccacgaccagtaccacctctggcaccgccaccgagaccgcctgtgcgacccctaccgccgtggccgtcacctttaacgagatcgccaccaccacctacggcgagaatgtttacattgttgggtccatctccgagctcgggaactgggataccagcaaagcagtggccctgagtgcgtccaagtatacctccagcaataacctctggtacgtgtccgtcaccctgccggctggcacgacattcgagtacaagtatatccgcaaggaaagcgatggctcgatcgtgtgggagagtgaccccaaccgctcgtatacggtgccggcagcttgtggagtgtctactgcgaccgagaatgatacttggcggtga

SEQ ID NO: 53 - DNA, 1896 bp, Aspergillus
  CDS (1)...(1896)
atgcctcgcctttcctacgcgctctgtgcgctgtctctcgggcatgctgctattgcagctcctcagttatccgctcgtgctaccggcagcttggactcctggttgggtactgagaccaccgttgcgctcaatggtattctggccaacatcggtgccgacggtgcttatgcgaagagcgctaagcctggcataatcattgccagtccgagcaccagcgaaccagactactactatacctggacgagagatgctgctctcgtcacgaaagtcctggtcgacctcttccgcaacggcaacctgggtctgcagaaagtcattaccgaatacgtcaactctcaggcgtacttgcagaccgtgtctaatccgtcgggtggtcttgcgagcggaggtctcgcggagcctaagtacaacgtcgacatgacggcctttaccggagcctggggtcgtcctcagcgtgatggtccggctctgcgggccaccgccctcatcgactttggcaactggctgattgacaacggctactccagctatgctgtcaacaacatctggcccattgtgcgcaacgacttgtcctacgtttctcagtactggagccagagtggctttgatctctgggaagaagtcaactccatgtccttcttcaccgtcgctgtccagcaccgtgccctcgtggagggaagcacgttcgctaaacgggtgggagcgtcgtgctcgtggtgtgactcgcaggccccccagatcctctgctacatgcagagtttctggactggctcgtatatcaacgccaacaccggtggtggccggtccggcaaggatgccaacaccgtcctcgccagcatccataccttcgaccccgaagccggctgcgacgatactactttccagccctgctctcctcgggcccttgccaaccacaaggtgtacaccgattcgttccgctcggtctacgcgatcaactccggcatcccacagggcgctgccgtttccgctggccgctaccccgaggacgtctactacaacggcaacccttggttcctcaccaccctcgccgctgccgagcagctctacgacgctatctaccagtggaagaagatcggttccatcagcatcaccagcacctccctcgccttcttcaaggacatctacagctccgccgcggtcggcacctacgcctctagcacctccaccttcacggacatcatcaacgcggtcaagacctacgcagacggctacgtgagcatcgtccaggcacacgccatgaacaacggctccctttcggagcaattcgacaagtcctctgggctgtccctctccgcccgcgatctgacctggtcctacgccgctttcctcaccgccaacatgcgtcgtaacggcgtggtgcctgccccctggggcgccgcctccgccaactccgtcccctcgtcttgctccatgggctcggccacgggcacctacagcaccgcgacagccacctcctggcccagcacgctgaccagcggctcgccaggcagcaccaccaccgtgggcaccacgaccagtaccacctctggcaccgccaccgagaccgcctgtgcgacccctaccgccgtggccgtcacctttaacgagatcgccaccaccacctacggcgagaatgtttacattgttgggtccatctccgagctcgggaactgggataccagcaaagcagtggccctgagtgcgtccaagtatacctccagcaataacctctggtacgtgtccgtcaccctgccggctggcacgacattcgagtacaagtatatccgcaaggaaagcgatggctcgatcgtgtgggagagtgaccccaaccgctcgtatacggtgccggcagcttgtggagtgtctactgcgaccgagaatgatacttggcggtga

SEQ ID NO: 54 - protein, 631 aa, Aspergillus
MPRLSYALCALSLGHAAIAAPQLSARATGSLDSWLGTETTVALNGILANIGADGAYAKSAKPGIIIASPSTSEPDYYYTWTRDAALVTKVLVDLFRNGNLGLQKVITEYVNSQAYLQTVSNPSGGLASGGLAEPKYNVDMTAFTGAWGRPQRDGPALRATALIDFGNWLIDNGYSSYAVNNIWPIVRNDLSYVSQYWSQSGFDLWEEVNSMSFFTVAVQHRALVEGSTFAKRVGASCSWCDSQAPQILCYMQSFWTGSYINANTGGGRSGKDANTVLASIHTFDPEAGCDDTTFQPCSPRALANHKVYTDSFRSVYAINSGIPQGAAVSAGRYPEDVYYNGNPWFLTTLAAAEQLYDAIYQWKKIGSISITSTSLAFFKDIYSSAAVGTYASSTSTFTDIINAVKTYADGYVSIVQAHAMNNGSLSEQFDKSSGLSLSARDLTWSYAAFLTANMRRNGVVPAPWGAASANSVPSSCSMGSATGTYSTATATSWPSTLTSGSPGSTTTVGTTTSTTSGTATETACATPTAVAVTFNEIATTTYGENVYIVGSISELGNWDTSKAVALSASKYTSSNNLWYVSVTLPAGTTFEYKYIRKESDGSIVWESDPNRSYTVPAACGVSTATENDTWR

SEQ ID NO: 55 - DNA, 1316 bp, Aspergillus
atgctgaaactgatgggttctcttgttctcctcgcgtctgcggccgaggtgattgcctctcccgcggctgagccagttgcgccgtccacaactctggagaagcgcgcaccttgcacgttttccgggtccaacggcgccgctgctgcgatggcttctcagaaggcttgctccactattgtcctgtcaaacgtggctgttccggctggcacgacgctggaccttagcgatctggcggatggcaccacagtacgaaccccatctccgacaccaaccccctaattcgtcgaggcaatagtctaattactccaggtcaccttcgagggcgagacaacctggggttaccaggagtggtccggtccactgctgaagatttccggcaagaacatcaaggtgaagggcgcgtcgggggctacgctgaaccccgacggcgcccgctggtgggacggccagggcggcaacggcggcaagacgaagcccaagttcttcgccgcgcacgatctcacctcgtcatcatccatcaccgatctgcacatcttgaacacccccgtccaggcggtcagcatcaacggatgcgatggcctgaccatcaccgacataacgatcgacaattccgccggagacacccaaggcggccacaacactgacgccttcgatattggatccagctccaacattatcatttcaggcgccaaggtctacaaccaggacgactgtgtcgcggtcaactccggcacggatatcacctttaccggggggctctgctccggtggccatggcctgtcgattggtagcgttggtggccggtctgataatactgtcgagaatgtgtcctttaccaactcccaggtgaccaactccgacaatggtaccatcacctccccccgttgtcttgtcgagggtaaacttctaaccatgaagcaggtctccgcatcaaggccaccaaagggaagactggcacaatcaagggagtcacctactcaggcatcaccctgagctccatccgcaagtcagctctcctcggcttcttcgtataaaagatgacggttatcaagctaacattgccaaggtacggtatcctcatcgagcaaaactacgacggcggcgatctcaagggcgacccgacgtccggcatccccatcaccgacctcaccatgcagaacatcagtggtaaaggcgccgtggcgtcaagcgggtacaatattgctattgtctgcggcagtggggcttgctcgaactggacctggaagagcgtcgaggttaccggcgggaagacctatggtagctgcaagaacgttcctagcgttgcccaatgctag

SEQ ID NO: 56 - DNA, 1137 bp, Aspergillus
  CDS (1)...(1137)
atgctgaaactgatgggttctcttgttctcctcgcgtctgcggccgaggtgattgcctctcccgcggctgagccagttgcgccgtccacaactctggagaagcgcgcaccttgcacgttttccgggtccaacggcgccgctgctgcgatggcttctcagaaggcttgctccactattgtcctgtcaaacgtggctgttccggctggcacgacgctggaccttagcgatctggcggatggcaccacagtcaccttcgagggcgagacaacctggggttaccaggagtggtccggtccactgctgaagatttccggcaagaacatcaaggtgaagggcgcgtcgggggctacgctgaaccccgacggcgcccgctggtgggacggccagggcggcaacggcggcaagacgaagcccaagttcttcgccgcgcacgatctcacctcgtcatcatccatcaccgatctgcacatcttgaacacccccgtccaggcggtcagcatcaacggatgcgatggcctgaccatcaccgacataacgatcgacaattccgccggagacacccaaggcggccacaacactgacgccttcgatattggatccagctccaacattatcatttcaggcgccaaggtctacaaccaggacgactgtgtcgcggtcaactccggcacggatatcacctttaccggggggctctgctccggtggccatggcctgtcgattggtagcgttggtggccggtctgataatactgtcgagaatgtgtcctttaccaactcccaggtgaccaactccgacaatggtctccgcatcaaggccaccaaagggaagactggcacaatcaagggagtcacctactcaggcatcaccctgagctccatccgcaagtacggtatcctcatcgagcaaaactacgacggcggcgatctcaagggcgacccgacgtccggcatccccatcaccgacctcaccatgcagaacatcagtggtaaaggcgccgtggcgtcaagcgggtacaatattgctattgtctgcggcagtggggcttgctcgaactggacctggaagagcgtcgaggttaccggcgggaagacctatggtagctgcaagaacgttcctagcgttgcccaatgctag

SEQ ID NO: 57 - protein, 378 aa, Aspergillus
MLKLMGSLVLLASAAEVIASPAAEPVAPSTTLEKRAPCTFSGSNGAAAAMASQKACSTIVLSNVAVPAGTTLDLSDLADGTTVTFEGETTWGYQEWSGPLLKISGKNIKVKGASGATLNPDGARWWDGQGGNGGKTKPKFFAAHDLTSSSSITDLHILNTPVQAVSINGCDGLTITDITIDNSAGDTQGGHNTDAFDIGSSSNIIISGAKVYNQDDCVAVNSGTDITFTGGLCSGGHGLSIGSVGGRSDNTVENVSFTNSQVTNSDNGLRIKATKGKTGTIKGVTYSGITLSSIRKYGILIEQNYDGGDLKGDPTSGIPITDLTMQNISGKGAVASSGYNIAIVCGSGACSNWTWKSVEVTGGKTYGSCKNVPSVAQC

SEQ ID NO: 58 - DNA, 1238 bp, Aspergillus
atgcgttctgttaagctttttggcctggctgcgctgggctccctcggcgctgctgcccctgctccgtctcgcgtgtcggatctgaccaagaggtcttcaacttgtaccttcaccgcagcctcccaggctaccgagagcgcttctggttgctccgagattgtcctggacaacatcgaggttcctgccggtgagacactggatctctcggatgttgatgatggaaccaccgtacgttaataccattgattatctccctgtctatctctctgccattgctcccgagctaatccccgaagatcgtcttcgaaggcaccaccacctttggctataaagagtggagcggccccttgatccgcttcggcggcaaggacatcaccatcaagcagaactccggcgctgtgattgacggagaaggctcccgctggtgggacggcgagggcaccaatggcggcaagaccaagcccaagttcatgtacgcgcacagcctcgaggactcgaccatcaccgggctatccatcaagaacacacctgtgcaggccattagtgttcaggcgaccaacctctacctgatcgacatcaccatcgataactccgacggggatgacaacggcgggcacaacactgacgggtttgacatcagcgagagtaccggggtgtatatccgcggcgctaccgtcaagaaccaggatgactgcattgccatcaactccggcgaggtatgctcccctgtctttttttttttttccccgcaggttggcattgaagttaattgactccagaacatcgaattctccggtggtacctgctctggcggccacggtctctccattggctcggttggcggtcgcgacgacaacaccgtcaagaacgtcaccatcaccgactccaccgtgaccgattcggccaacggcgtgcgtatcaagacggtctacgacgccaccggctctgttagccaagtcacctactccaacatcaagctgtcaggtatcacggactatggtatcgtcatcgagcaggactacgagaacggcagcccgaccggtacccctaccactggcgttccgatcaccgacctgactatcgacggtgtgactggtaccgtcgagtccgacgccgtcgaggtgtacattctttgcggagacggtagctgcagtgactggacctgggagggcgtggatatcactggcggggagaagagctccaagtgcgagaatgttccctcaggtgcttcttgctag

SEQ ID NO: 59 - DNA, 1107 bp, Aspergillus
  CDS (1)...(1107)
atgcgttctgttaagctttttggcctggctgcgctgggctccctcggcgctgctgcccctgctccgtctcgcgtgtcggatctgaccaagaggtcttcaacttgtaccttcaccgcagcctcccaggctaccgagagcgcttctggttgctccgagattgtcctggacaacatcgaggttcctgccggtgagacactggatctctcggatgttgatgatggaaccaccatcgtcttcgaaggcaccaccacctttggctataaagagtggagcggccccttgatccgcttcggcggcaaggacatcaccatcaagcagaactccggcgctgtgattgacggagaaggctcccgctggtgggacggcgagggcaccaatggcggcaagaccaagcccaagttcatgtacgcgcacagcctcgaggactcgaccatcaccgggctatccatcaagaacacacctgtgcaggccattagtgttcaggcgaccaacctctacctgatcgacatcaccatcgataactccgacggggatgacaacggcgggcacaacactgacgggtttgacatcagcgagagtaccggggtgtatatccgcggcgctaccgtcaagaaccaggatgactgcattgccatcaactccggcgagaacatcgaattctccggtggtacctgctctggcggccacggtctctccattggctcggttggcggtcgcgacgacaacaccgtcaagaacgtcaccatcaccgactccaccgtgaccgattcggccaacggcgtgcgtatcaagacggtctacgacgccaccggctctgttagccaagtcacctactccaacatcaagctgtcaggtatcacggactatggtatcgtcatcgagcaggactacgagaacggcagcccgaccggtacccctaccactggcgttccgatcaccgacctgactatcgacggtgtgactggtaccgtcgagtccgacgccgtcgaggtgtacattctttgcggagacggtagctgcagtgactggacctgggagggcgtggatatcactggcggggagaagagctccaagtgcgagaatgttccctcaggtgcttcttgctag

SEQ ID NO: 60 - protein, 368 aa, Aspergillus
MRSVKLFGLAALGSLGAAAPAPSRVSDLTKRSSTCTFTAASQATESASGCSEIVLDNIEVPAGETLDLSDVDDGTTIVFEGTTTFGYKEWSGPLIRFGGKDITIKQNSGAVIDGEGSRWWDGEGTNGGKTKPKFMYAHSLEDSTITGLSIKNTPVQAISVQATNLYLIDITIDNSDGDDNGGHNTDGFDISESTGVYIRGATVKNQDDCIAINSGENIEFSGGTCSGGHGLSIGSVGGRDDNTVKNVTITDSTVTDSANGVRIKTVYDATGSVSQVTYSNIKLSGITDYGIVIEQDYENGSPTGTPTTGVPITDLTIDGVTGTVESDAVEVYILCGDGSCSDWTWEGVDITGGEKSSKCENVPSGASC

SEQ ID NO: 61 - DNA, 1149 bp, Aspergillus
atgcatttcttccaatcctccctcgtggccgcgaccatgggcgctgcgctggtcgctgccgcacctgccgctgatctcgagactcgcggctcttgcactttcacctctacgtccgcgctcaagtccggcaaggcctcgtgctccaccatcactctccagaacatcgcggtgcccgcgggtgagaccctggacctgactggcctgaaggccggtaccactgtaagtcttacccttgaattgtctgaatgccatcggctgatgtgctgccaaaaggtcgtcttcgacggtaccaccaccttcggctacaaggaatgggagggccccctgatttccgcgtccggaaccagtattaccatcaagcagaaccctggtgctaagattgactgtgatggtgctcgttggtgggacggcaagggaggcaacggtggcaagaagaagcccaagttcttctctgcccacaagctgaacaagtcgaacatcaccggcctgaaggtctacaacacccccgtccatggcttcagcatccagtccgaccacctcaccatcaaggacgtcctcctcgacaactccgccggtaccaagctcggccacaataccgatgcctttgacgtcggttccagcacctacatcactattgacggcgccaccgtctacaaccaggacgactgcctggccgtcaactccggtgagcacatcaccttcaccaacggctactgcaacggtggtcacggtctctccatcggctccgtcggcggccgcagtaacaatgtcgtcaacgacgtcaccatctccaactcccaggtcatcaactcccagaacggtgctcggatcaagaccgtctacggcgcgactggctctgtcaccggcgtcaagttccaggatatctccctcaaaggtatcaccaagtacggtattgttgtccagcaggactacgagaacggcaagcccactggcaagcccaccaacggcgtcaaggtctccgatatcaccttcgagaaggtcaccggtaccgtcaccagctccgccactgatatttacattctctgcggatctggcagctgcaccaactggacctggtctggcaatagcgtcaccggtggcaagaagagctccagttgcaagaacgttcctgctggcgcctcatgctag

SEQ ID NO: 62 - DNA, 1095 bp, Aspergillus
  CDS (1)...(1095)
atgcatttcttccaatcctccctcgtggccgcgaccatgggcgctgcgctggtcgctgccgcacctgccgctgatctcgagactcgcggctcttgcactttcacctctacgtccgcgctcaagtccggcaaggcctcgtgctccaccatcactctccagaacatcgcggtgcccgcgggtgagaccctggacctgactggcctgaaggccggtaccactgtcgtcttcgacggtaccaccaccttcggctacaaggaatgggagggccccctgatttccgcgtccggaaccagtattaccatcaagcagaaccctggtgctaagattgactgtgatggtgctcgttggtgggacggcaagggaggcaacggtggcaagaagaagcccaagttcttctctgcccacaagctgaacaagtcgaacatcaccggcctgaaggtctacaacacccccgtccatggcttcagcatccagtccgaccacctcaccatcaaggacgtcctcctcgacaactccgccggtaccaagctcggccacaataccgatgcctttgacgtcggttccagcacctacatcactattgacggcgccaccgtctacaaccaggacgactgcctggccgtcaactccggtgagcacatcaccttcaccaacggctactgcaacggtggtcacggtctctccatcggctccgtcggcggccgcagtaacaatgtcgtcaacgacgtcaccatctccaactcccaggtcatcaactcccagaacggtgctcggatcaagaccgtctacggcgcgactggctctgtcaccggcgtcaagttccaggatatctccctcaaaggtatcaccaagtacggtattgttgtccagcaggactacgagaacggcaagcccactggcaagcccaccaacggcgtcaaggtctccgatatcaccttcgagaaggtcaccggtaccgtcaccagctccgccactgatatttacattctctgcggatctggcagctgcaccaactggacctggtctggcaatagcgtcaccggtggcaagaagagctccagttgcaagaacgttcctgctggcgcctcatgctag

SEQ ID NO: 63 - protein, 364 aa, Aspergillus
MHFFQSSLVAATMGAALVAAAPAADLETRGSCTFTSTSALKSGKASCSTITLQNIAVPAGETLDLTGLKAGTTVVFDGTTTFGYKEWEGPLISASGTSITIKQNPGAKIDCDGARWWDGKGGNGGKKKPKFFSAHKLNKSNITGLKVYNTPVHGFSIQSDHLTIKDVLLDNSAGTKLGHNTDAFDVGSSTYITIDGATVYNQDDCLAVNSGEHITFTNGYCNGGHGLSIGSVGGRSNNVVNDVTISNSQVINSQNGARIKTVYGATGSVTGVKFQDISLKGITKYGIVVQQDYENGKPTGKPTNGVKVSDITFEKVTGTVTSSATDIYILCGSGSCTNWTWSGNSVTGGKKSSSCKNVPAGASC

SEQ ID NO: 64 - DNA, 712 bp, Aspergillus
atggtctcattctcttctctcgttctcgctgcctccaccgttgctggcgtgctagctacacccggctcggagcaatacgttgagctagccaagcggcagctcaccagctctcagactggcacgaataacggctactactactccttctggaccgacggcggcggccaggtgacctacaccaacggcaatggcggccagtatcaggtcgactggaacaactgcggcaactttgttgctgggaagggctggaacccggccagcgagaagtatgcgtcctctccctgcttgttaggttcaagctaatggattcagagcggtcacctacagcggctcctggcagaccagcggaaacggctacctctccgtgtacggctggacgaccagtccgctggtcgaattctacatcgtggagagttacggctcctatgacccctccacgggagccacccatctcggcaccgtcgagagcgacggggccacgtacaacctctacaagacgacgcggacgaatgcgccgtccatccagggcacggctacttttgaccagtactggtcggttcggacttcgcaccggcagagtggaactgtgacgacgaagaaccactttgatgcgtggagaaatgcgggtctgcaattggggaactttgactatatgattgttgcgacggaggggtaccagagcagcggctctgctactatcactgtttcttag

SEQ ID NO: 65 - DNA, 666 bp, Aspergillus
  CDS (1)...(666)
atggtctcattctcttctctcgttctcgctgcctccaccgttgctggcgtgctagctacacccggctcggagcaatacgttgagctagccaagcggcagctcaccagctctcagactggcacgaataacggctactactactccttctggaccgacggcggcggccaggtgacctacaccaacggcaatggcggccagtatcaggtcgactggaacaactgcggcaactttgttgctgggaagggctggaacccggccagcgagaaagcggtcacctacagcggctcctggcagaccagcggaaacggctacctctccgtgtacggctggacgaccagtccgctggtcgaattctacatcgtggagagttacggctcctatgacccctccacgggagccacccatctcggcaccgtcgagagcgacggggccacgtacaacctctacaagacgacgcggacgaatgcgccgtccatccagggcacggctacttttgaccagtactggtcggttcggacttcgcaccggcagagtggaactgtgacgacgaagaaccactttgatgcgtggagaaatgcgggtctgcaattggggaactttgactatatgattgttgcgacggaggggtaccagagcagcggctctgctactatcactgtttcttag

SEQ ID NO: 66 - protein, 221 aa, Aspergillus
MVSFSSLVLAASTVAGVLATPGSEQYVELAKRQLTSSQTGTNNGYYYSFWTDGGGQVTYTNGNGGQYQVDWNNCGNFVAGKGWNPASEKAVTYSGSWQTSGNGYLSVYGWTTSPLVEFYIVESYGSYDPSTGATHLGTVESDGATYNLYKTTRTNAPSIQGTATFDQYWSVRTSHRQSGTVTTKNHFDAWRNAGLQLGNFDYMIVATEGYQSSGSATITVS

SEQ ID NO: 67 - DNA, 739 bp, Aspergillus
atggtttctttctcctacctgctgctggcgtgctccgccattggagctctggctgcccccgtcgaacccgagaccacctcgttcaatgagactgctcttcatgagttcgctgagcgcgccggcaccccaagctccaccggctggaacaacggctactactactccttctggactgatggcggcggcgacgtgacctacaccaatggcgccggtggctcgtactccgtcaactggaggaacgtgggcaactttgtcggtggaaagggctggaaccctggaagcgctaggtaccgagctttgtcaacgtcggatgtgcagacctgtggctgacagaagtagaaccatcaactacggaggcagcttcaaccccagcggcaatggctacctggctgtctacggctggaccaccaaccccttgattgagtactacgttgttgagtcgtatggtacatacaaccccggcagcggcggtaccttcaggggcactgtcaacaccgacggtggcacttacaacatctacacggccgttcgctacaatgctccctccatcgaaggcaccaagaccttcacccagtactggtctgtgcgcacctccaagcgtaccggcggcactgtcaccatggccaaccacttcaacgcctggagcagactgggcatgaacctgggaactcacaactaccagattgtcgccactgagggttaccagagcagcggatctgcttccatcactgtctactag

SEQ ID NO: 68 - DNA, 705 bp, Aspergillus
  CDS (1)...(705)
atggtttctttctcctacctgctgctggcgtgctccgccattggagctctggctgcccccgtcgaacccgagaccacctcgttcaatgagactgctcttcatgagttcgctgagcgcgccggcaccccaagctccaccggctggaacaacggctactactactccttctggactgatggcggcggcgacgtgacctacaccaatggcgccggtggctcgtactccgtcaactggaggaacgtgggcaactttgtcggtggaaagggctggaaccctggaagcgctaggtaccgagctttaagtagaaccatcaactacggaggcagcttcaaccccagcggcaatggctacctggctgtctacggctggaccaccaaccccttgattgagtactacgttgttgagtcgtatggtacatacaaccccggcagcggcggtaccttcaggggcactgtcaacaccgacggtggcacttacaacatctacacggccgttcgctacaatgctccctccatcgaaggcaccaagaccttcacccagtactggtctgtgcgcacctccaagcgtaccggcggcactgtcaccatggccaaccacttcaacgcctggagcagactgggcatgaacctgggaactcacaactaccagattgtcgccactgagggttaccagagcagcggatctgcttccatcactgtctactag

SEQ ID NO: 69 - protein, 234 aa, Aspergillus
MVSFSYLLLACSAIGALAAPVEPETTSFNETALHEFAERAGTPSSTGWNNGYYYSFWTDGGGDVTYTNGAGGSYSVNWRNVGNFVGGKGWNPGSARYRALSRTINYGGSFNPSGNGYLAVYGWTTNPLIEYYVVESYGTYNPGSGGTFRGTVNTDGGTYNIYTAVRYNAPSIEGTKTFTQYWSVRTSKRTGGTVTMANHFNAWSRLGMNLGTHNYQIVATEGYQSSGSASITVY

SEQ ID NO: 70 - DNA, 1002 bp, Aspergillus
atgatctccatttcctcgctcagctttggactcgccgctatcgccggcgcatatgctcttccgagtgacaaatccgtcagcttagcggaacgtcagacgatcacgaccagccagacaggcacaaacaatggctactactattccttctggaccaacggtgccggatcagtgcaatatacaaatggtgctggtggcgaatatagtgtgacgtgggcgaaccagaacggtggtgactttacctgtgggaagggctggaatccagggagtgaccagtaggcaacgccccagaactatagaagaggacgcaaagaaagcactaaactctctactagtgacattaccttctctggcagcttcaatccttccggaaatgcttacctgtccgtgtatggatggactaccaaccccctagtcgaatactacatcctcgagaactatggcagttacaatcctggctcgggcatgacgcacaagggcaccgtcaccagcgatggatccacctacgacatctatgagcaccaacaggtcaaccagccttcgatcgtcggcacggccaccttcaaccaatactggtccatccgccaaaacaagcgatccagcggcacagtcaccaccgcgaatcacttcaaggcctgggctagtctggggatgaacctgggtacccataactatcagattgtttccactgagggatatgagagcagcggtacctcgaccatcactgtctcgtctggtggttcttcttctggtggaagtggtggcagctcgtctactacttcctcaggcagctcccctactggtggctccggcagtgtaagtcttcttccatatggttgtggctttatgtgtattctgactgtgatagtgctctgctttgtggggccagtgcggtggaattggctggtctggtcctacttgctgctcttcgggcacttgccaggtttcgaactcgtactactcccagtgcttgtagtaccttcttgcagggttatatccaagtga

SEQ ID NO: 71 - DNA, 942 bp, Aspergillus
  CDS (1)...(942)
atgatctccatttcctcgctcagctttggactcgccgctatcgccggcgcatatgctcttccgagtgacaaatccgtcagcttagcggaacgtcagacgatcacgaccagccagacaggcacaaacaatggctactactattccttctggaccaacggtgccggatcagtgcaatatacaaatggtgctggtggcgaatatagtgtgacgtgggcgaaccagaacggtggtgactttacctgtgggaagggctggaatccagggagtgaccatgacattaccttctctggcagcttcaatccttccggaaatgcttacctgtccgtgtatggatggactaccaaccccctagtcgaatactacatcctcgagaactatggcagttacaatcctggctcgggcatgacgcacaagggcaccgtcaccagcgatggatccacctacgacatctatgagcaccaacaggtcaaccagccttcgatcgtcggcacggccaccttcaaccaatactggtccatccgccaaaacaagcgatccagcggcacagtcaccaccgcgaatcacttcaaggcctgggctagtctggggatgaacctgggtacccataactatcagattgtttccactgagggatatgagagcagcggtacctcgaccatcactgtctcgtctggtggttcttcttctggtggaagtggtggcagctcgtctactacttcctcaggcagctcccctactggtggctccggcagtgtaagtcttcttccatatggttgtggctttatgtgtattctgactgtgatagtgctctgctttgtggggccagtgcggtggaattggctggtctggtcctacttgctgctcttcgggcacttgccaggtttcgaactcgtactactcccagtgcttgtagtaccttcttgcagggttatatccaagtga

SEQ ID NO: 72 - protein, 313 aa, Aspergillus
MISISSLSFGLAAIAGAYALPSDKSVSLAERQTITTSQTGTNNGYYYSFWTNGAGSVQYTNGAGGEYSVTWANQNGGDFTCGKGWNPGSDHDITFSGSFNPSGNAYLSVYGWTTNPLVEYYILENYGSYNPGSGMTHKGTVTSDGSTYDIYEHQQVNQPSIVGTATFNQYWSIRQNKRSSGTVTTANHFKAWASLGMNLGTHNYQIVSTEGYESSGTSTITVSSGGSSSGGSGGSSSTTSSGSSPTGGSGSVSLLPYGCGFMCILTVIVLCFVGPVRWNWLVWSYLLLFGHLPGFELVLLPVLVVPSCRVISK